(19) United States
(12) Patent Application Publication
(10) Pub. No.: US 2026/0137383 A1
(43) Pub. Date: May 21, 2026

(54) SUTURE CLOSURE DEVICES

(71) Applicant: THE GLOBAL HEART VALVE INNOVATION CENTER (ISREAL) LTD., Or Yehuda (IL)

(72) Inventors: Meni IAMBERGER, Kfar Saba (IL); Nir LILACH, Kfar Yehoshua (IL); Amit SEGAL, Afula (IL); Eliahu ELIACHAR, Haifa (IL); Ohad Yehoshua HENN, Ramat Gan (IL); Oded ALDAAG, Motza Ilit (IL)

(21) Appl. No.: 19/448,156

(22) Filed: Jan. 14, 2026

Related U.S. Application Data
(63) Continuation of application No. PCT/IL2024/050688, filed on Jul. 14, 2024.
Application No. PCT/IL2024/050688 is a continuation-in-part of application No. PCT/IL2023/050966, filed on Sep. 7, 2023.
(60) Provisional application No. 63/404,630, filed on Sep. 8, 2022.
(60) Provisional application No. 63/526,765, filed on Jul. 14, 2023.
(60) Provisional application No. 63/526,765, filed on Jul. 14, 2023.
(60) Provisional application No. 63/564,785, filed on Mar. 13, 2024.

(30) Foreign Application Priority Data
Sep. 7, 2023 (WO) PCT/IL2023/050966

(51) Int. Cl. A61B 17/04; A61B 17/06

(52) U.S. Cl. CPC A61B 17/0482; A61B 17/06004

(57) ABSTRACT

A closure device for deploying sutures includes a suture, an elongate support, and a suture-positioning support coupled to a distal end portion of the elongate support. The closure device is configured to transition the suture-positioning support from a delivery position to a first deployed position, in which the suture-positioning support laterally extends in a first direction; couple the suture to the suture-positioning support; thereafter, transition the suture-positioning support from the first deployed position back to the delivery position; thereafter, rotate the suture-positioning support, while in the delivery position, about an axis of the elongate support; thereafter, transition the suture-positioning support from the delivery position to a second deployed position, in which the suture-positioning support laterally extends in a second direction; and proximally withdraw the suture from the suture-positioning support while the suture-positioning support is in the second deployed position. Other embodiments are also described.

### CROSS-REFERENCE TO RELATED APPLICATIONS

The present disclosure is a Continuation Application of PCT Application No. PCT/IL2024/050688, filed on Jul. 14, 2024, which (i) claims priority from U.S. Provisional Application 63/526,765, filed Jul. 14, 2023, and U.S. Provisional Application 63/564,785, filed Mar. 13, 2024; and (ii) claims priority from and is a continuation-in-part of International Application PCT/IL 2023/050966, filed Sep. 7, 2023, which published as PCT Publication WO 2024/052916 to Iamberger et al., which claims priority from U.S. Provisional Application 63/404,630, filed Sep. 8, 2022, and US Provisional Application 63/526,765, filed Jul. 14, 2023. All of the above-mentioned applications are assigned to the assignee of the present application and incorporated herein by reference.

### FIELD OF THE APPLICATION

The present invention relates generally to closure devices, and specifically to suture closure devices.

### BACKGROUND OF THE APPLICATION

Vascular access for diagnostic and interventional vascular procedures is usually established using the Seldinger technique. Suture-applying devices are sometimes used to close the vascular access site upon completion of the procedure. These devices include a shaft that is inserted into the blood vessel through the vascular puncture, and one or more needles that are used to draw a suture through the blood vessel wall on opposite sides of the puncture. The suture is then secured outside the blood vessel to provide closure.

Closure devices are also used to deploy sutures within the body of a patient, such as to close surgical punctures, including endoscopic port site punctures. The devices are used to draw a suture through the fascia, muscle, and peritoneum layers of the endoscopic port site puncture on opposite sides of the puncture. The suture is then secured outside the fascia to close the fascia and peritoneum.

US Patent Application Publication 2013/0310856 to Sherts et al. describes suture passer guides and related kits and methods. In certain aspects, a suture passer guide includes an elongate member and a suture positioning member that can be radially extended from a distal end region of the elongate member and rotated relative to the elongate member such that the suture positioning member can reposition a suture from a first side area of the elongate member to a second side area of the elongate member.

US Patent Application Publication 2008/0045979 to Ma describes devices, systems, and methods for suturing of body lumens to allow the suturing of vascular puncture sites located at the distal end of a percutaneous tissue tract. An elongated articulated foot of the device can be inserted through the penetration and actuated so that the foot extends along the lumenal axis. The foot can carry suturing attachment cuffs with one end of the cuff adapted to receive a needle, while the other end receives suture. A portion of the foot and/or lumen of the shaft can receive a portion of the suture and can include friction reducing structure that aid with movement of the sutured during removal of the cuffs from within the penetration.

### SUMMARY OF THE APPLICATION

Embodiments of the present invention provide closure devices for suturing a puncture through a wall of a hollow anatomical structure, such as a blood vessel, at an access site. In some embodiments of the present invention, the closure devices comprises one or more sutures; one or more ferrules, coupled to distal end portions of the one or more sutures, respectively; and an elongate support. A suture-positioning support is laterally extendable from a distal portion of the shaft, and is shaped so as to define a ferrule receptacle, which is configured to removably receive the one or more ferrules. A suturing needle is removably couplable to each of the one or more ferrules. The closure devices are configured to direct the one or more ferrules into the ferrule receptacle during distal advancement of the respective suturing needles removably coupled to the respective ferrules.

In some embodiments of the present invention, methods are also provided for using the closure devices for suturing the puncture. The distal portion of the shaft inserted through the puncture and into the hollow anatomical structure while the suture-positioning support is in a delivery position. The suture-positioning support is transitioned from the delivery position to a first deployed position in which the suture-positioning support laterally extends in a first direction from the distal portion of the shaft. One of the one or more suturing needles is distally advanced through a first wall site of the wall and into the hollow anatomical structure, while the suturing needle is removably coupled to the ferrule, such that the closure device directs one of the one or more ferrules into the ferrule receptacle defined by the suture-positioning support and the ferrule receptacle removably receives the ferrule while the suture-positioning support is in the first deployed position. The suturing needle is proximally withdrawn from the ferrule and the hollow anatomical structure, while leaving the ferrule within the ferrule receptacle.

The suture-positioning support is transitioned from the first deployed position to a second deployed position in which the suture-positioning support laterally extends in a second direction from the distal portion of the shaft, the second direction different from the first direction. The ferrule is proximally withdrawn from the ferrule receptacle and out of the hollow anatomical structure via a second wall site of the wall, so as to proximally withdraw a portion of the suture, including the distal end portion thereof, out of the hollow anatomical structure via the second wall site. The portion of the suture drawn out of the hollow anatomical structure via the second wall site is secured to another portion of the suture outside the hollow anatomical structure.

According to one aspect, embodiments of the present invention provides a closure device for deploying sutures, the closure device includes:
a suture;
an elongate support; and
a suture-positioning support, which is coupled to a distal end portion of the elongate support, and which is configured to assume:
(i) a delivery position, in which a suture-positioning support axis of the suture-positioning support (a) forms a first angle of less than 45 degrees with a distal-support central longitudinal axis of the distal end portion of the elongate support, or (b) is parallel with the distal-support central longitudinal axis, and
(ii) a plurality of deployed positions, in which the suture-positioning support is laterally extended with respect to the distal end portion of the elongate support such that the suture-positioning support axis forms a second angle of at least 60 degrees with the distal-support central longitudinal axis,
wherein the closure device is configured to:
transition the suture-positioning support from the delivery position to a first one of the deployed positions, in which the suture-positioning support laterally extends in a first direction from the distal end portion of the elongate support,
couple the suture to the suture-positioning support while the suture-positioning support is in the first deployed position,
thereafter, transition the suture-positioning support from the first deployed position back to the delivery position,
thereafter, rotate the suture-positioning support, while in the delivery position, about a distal-support central longitudinal axis of the distal portion of the elongate support,
thereafter, transition the suture-positioning support from the delivery position to a second one of the deployed positions, in which the suture-positioning support laterally extends in a second direction from the distal end portion of the elongate support, the second direction different from the first direction, and
proximally withdraw the suture from the suture-positioning support while the suture-positioning support is in the second deployed position.

According to another aspect, embodiments of the present invention further provide another the closure device for deploying sutures, the closure device includes:
a suture;
a ferrule, coupled to an end portion of the suture;
an elongate support, which includes one or more shafts;
a suture-positioning support, which is (a) coupled to a distal end portion of the elongate support, (b) laterally extendable with respect to the elongate support, and (c) shaped so as to define a ferrule receptacle, which is configured to removably receive the ferrule;
a control handle, which (a) is coupled to a proximal end portion of the elongate support, and (b) includes a wall shaped so as to define a lateral opening through the wall; and
a ferrule-withdrawing suturing needle, which (a) includes a distal end portion that is removably couplable to the ferrule, and (b) is removably disposed entirely within the control handle,
wherein the closure device is configured such that:
distal advancement of the ferrule-withdrawing suturing needle to the ferrule in the ferrule receptacle couples the ferrule-withdrawing suturing needle to the ferrule,
proximal withdrawal of the ferrule-withdrawing suturing needle coupled to the ferrule removes the ferrule from the ferrule receptacle, and
the ferrule-withdrawing suturing needle is removable from the control handle via the lateral opening.

According to yet another aspect, embodiments of the present disclosure furthermore provide an apparatus including:
a suture;
a ferrule-withdrawing suturing needle;
a ferrule, which (a) is coupled to a distal end portion of the suture, and (b) is shaped so as to define (i) a ferrule lumen and (ii) one or more tabs, which are biased to protrude radially inward within the ferrule lumen and to engage the ferrule-withdrawing suturing needle upon insertion of the ferrule-withdrawing suturing needle into an end opening of the ferrule lumen, thereby inhibiting withdrawal of the ferrule-withdrawing suturing needle from the ferrule lumen; and
a ferrule-advancing suturing needle, which includes a distal end portion that is shaped and sized so as, upon insertion of the distal end portion into the ferrule lumen, to provide space within the ferrule lumen for the one or more tabs, and to contact an internal surface of the ferrule lumen so as to grasp the ferrule.

The present invention will be more fully understood from the following detailed description of embodiments thereof, taken together with the drawings, in which:

### BRIEF DESCRIPTION OF THE DRAWINGS

FIG. 1A is a schematic illustration of still another closure device for suturing a puncture with at least one suture, in accordance with an application of the present invention;

FIG. 1B is a schematic illustration of a distal end portion of an elongate support of the closure device of FIG. 1A, in accordance with an application of the present invention;

FIGS. 1C-D are schematic illustrations of distal portions of suturing needles and sutures of the closure device of FIG. 1A, from two views, in accordance with an application of the present invention;

FIGS. 2A-D are several schematic views of a portion of the closure device of FIG. 1A, in accordance with an application of the present invention;

FIGS. 3A-B are schematic illustrations of a suture-positioning support of the closure device of FIG. 1A laterally extended from a distal end portion of an elongate support of the closure device of FIG. 1A, in accordance with an application of the present invention;

FIG. 4 is a schematic cross-sectional view of a portion of the closure device of FIG. 1A, in accordance with an application of the present invention;

FIGS. 5A-R are schematic illustrations of a method for suturing a puncture through a wall of a hollow anatomical structure using the closure device of FIG. 1A, in accordance with an application of the present invention;

FIGS. 6A-B are schematic illustrations of a configuration of the closure device of FIG. 1A, including a control handle thereof, in accordance with an application of the present invention;

FIGS. 7A-H are schematic cross-sectional illustrations of a method of using the control handle of the closure device configuration of FIGS. 6A-B, in accordance with an application of the present invention;

FIGS. 8A and 8B are schematic isometric and cross-sectional views of an alternative configuration of a proximal end portion of a dilator of the closure device of FIG. 1A, with a sheath distally advanced, in accordance with an application of the present invention;

FIGS. 9A and 9B are schematic isometric and cross-sectional views of the alternative configuration of the proximal end portion of the dilator of FIGS. 8A and 8B, with the sheath proximally withdrawn, in accordance with an application of the present invention;

FIGS. 10A and 10B are schematic cross-sectional illustrations of respective configurations of a sheath, an elongate support, and a portion of a control handle of the closure device of FIG. 1A, in accordance with respective applications of the present invention;

FIGS. 11A-C are schematic illustrations of an alternative configuration of a suture-positioning support of the closure device of FIG. 1A, in accordance with an application of the present invention;

FIGS. 11D-F are schematic illustrations of the alternative configuration of suture-the positioning support shown in FIGS. 11A-C, as well as a distal end portion of an elongate support, an elongate flexible dilator connector, and a portion of a proximal end portion of a dilator of the closure device of FIG. 1A, in accordance with an application of the present invention;

FIGS. 12A-C are schematic illustrations of another alternative configuration of a suture-positioning support of the closure device of FIG. 1A, in accordance with an application of the present invention;

FIGS. 12D-E are schematic illustrations of the alternative configuration of suture-the positioning support shown in FIGS. 12A-C, as well as a distal end portion of an elongate support, an elongate flexible dilator connector, and a portion of a proximal end portion of a dilator of the closure device of FIG. 1A, in accordance with an application of the present invention;

FIG. 12F is a schematic illustration of the alternative configuration of the suture-positioning support of FIGS. 11A-F, as well as a distal end portion of an elongate support, an elongate flexible dilator connector, and a portion of a proximal end portion of a dilator of the closure device of FIG. 1A, in accordance with an application of the present invention;

FIGS. 13A-C are schematic isometric and cross-sectional illustrations of pre-tied knots, in accordance with an application of the present invention;

FIGS. 14A-C are schematic isometric and cross-sectional illustrations of additional pre-tied knots, in accordance with an application of the present invention;

FIGS. 15A-C are schematic illustrations of couplings between a distal end portion of a suture and a ferrule, in accordance with an application of the present invention;

FIGS. 16A-C are schematic illustrations of additional couplings between a distal end portion of a suture and a ferrule, in accordance with an application of the present invention;

FIGS. 17A-C are schematic illustrations of a suture, in accordance with an application of the present invention;

FIGS. 18A-B are schematic isometric and cross-sectional views of a crimping technique using a crimp tube, in accordance with an application of the present invention;

FIGS. 19A-B and 19C-D are schematic isometric and cross-sectional views of additional crimping techniques using a crimp tube, in accordance with respective applications of the present invention;

FIG. 19E is a schematic illustration of another fixation of distal and proximal suture segments of a suture, in accordance with an application of the present invention;

FIGS. 20A-B are schematic illustrations of another configuration of the closure device of FIG. 1A, in accordance with an application of the present invention;

FIGS. 21A-E are schematic illustrations of a ferrule-advancing suturing needle, in accordance with an application of the present invention;

FIGS. 21F-J are schematic illustrations of another ferrule-advancing suturing needle, in accordance with an application of the present invention;

FIGS. 22A-D are schematic illustrations of another ferrule-withdrawing suturing needle, in accordance with an application of the present invention;

FIGS. 23A-D are schematic illustrations of yet another ferrule-withdrawing suturing needle, in accordance with an application of the present invention;

FIGS. 24A-D are schematic illustrations of still another ferrule-withdrawing suturing needle, in accordance with an application of the present invention;

FIGS. 25A-C are schematic illustrations of a configuration of a control handle of the closure device of FIG. 1A, in accordance with an application of the present invention;

FIGS. 26A-B are schematic cross-sectional illustrations of another configuration of a control handle of the closure device of FIG. 1A, in accordance with an application of the present invention;

FIGS. 27A-F are schematic cross-sectional illustrations of a portion of an alternative control handle of the closure device of FIG. 1A, and a method of using the alternative control handle, in accordance with an application of the present invention;

FIGS. 28A-B are schematic illustrations of a needle-advancement safety control assembly, in accordance with an application of the present invention;

FIG. 29A is a schematic illustration of a cutting blade assembly of a control handle of the closure device of FIG. 1A, in accordance with an application of the present invention;

FIG. 29B is a schematic illustration of another configuration of the control handle of the closure device of FIG. 1A, in accordance with an application of the present invention;

FIGS. 30A-C are schematic illustrations of a ferrule, a portion of a ferrule-advancing suturing needle, and a portion of a suture, in accordance with an application of the present invention;

FIGS. 31A-C are schematic illustrations of a ferrule, a portion of a ferrule-advancing suturing needle, and a portion of a suture, in accordance with an application of the present invention;

FIGS. 32A-D are schematic illustrations of a ferrule, a portion of a ferrule-advancing suturing needle, and a portion of a suture, in accordance with an application of the present invention;

FIG. 33 is a schematic illustration of a ferrule, a portion of a ferrule-advancing suturing needle, and a portion of a suture, in accordance with an application of the present invention; and

FIGS. 34A-F are schematic cross-sectional illustrations of an alternative control handle of a closure device, and a method of using the alternative control handle, in accordance with an application of the present invention.

### DETAILED DESCRIPTION OF APPLICATIONS

In addition to the embodiments described in the above summary, there is furthermore provided, in accordance with an Inventive Concept 1 of the present invention, a closure device for suturing a puncture, the closure device including:
a suture;
an elongate support; and

a suture-positioning support, which is coupled to a distal end portion of the elongate support, and which is configured to assume:
(i) a delivery position, in which a suture-positioning support axis of the suture-positioning support (a) forms a first angle of less than 45 degrees with a distal-support central longitudinal axis of the distal end portion of the elongate support, or (b) is parallel with the distal-support central longitudinal axis, and
(ii) a plurality of deployed positions, in which the suture-positioning support is laterally extended with respect to the distal end portion of the elongate support such that the suture-positioning support axis forms a second angle of at least 60 degrees with the distal-support central longitudinal axis,
wherein the closure device is configured to:
transition the suture-positioning support from the delivery position to a first one of the deployed positions, in which the suture-positioning support laterally extends in a first direction from the distal end portion of the elongate support,
couple the suture to the suture-positioning support while the suture-positioning support is in the first deployed position,
thereafter, transition the suture-positioning support from the first deployed position back to the delivery position,
thereafter, rotate the suture-positioning support, while in the delivery position, about a distal-support central longitudinal axis of the distal portion of the elongate support,
thereafter, transition the suture-positioning support from the delivery position to a second one of the deployed positions, in which the suture-positioning support laterally extends in a second direction from the distal end portion of the elongate support, the second direction different from the first direction, and
proximally withdraw the suture from the suture-positioning support while the suture-positioning support is in the second deployed position.

Inventive Concept 2. The closure device according to Inventive Concept 1, wherein the first angle is less than degrees.

Inventive Concept 3. The closure device according to Inventive Concept 1, wherein the puncture is a puncture through a wall of a blood vessel.

Inventive Concept 4. The closure device according to Inventive Concept 1, wherein the puncture is an endoscopic puncture through a wall of a body cavity.

Inventive Concept 5. The closure device according to Inventive Concept 1, wherein an external portion of the suture-positioning support that interfaces with the distal end portion of the elongate support is shaped so as to define two partial spherical surfaces.

Inventive Concept 6. The closure device according to Inventive Concept 1, wherein the elongate support includes one or more shafts, and wherein the elongate support is configured to rotate the suture-positioning support about the distal-support central longitudinal axis by rotating at least one of the one or more of the shafts of the elongate support.

Inventive Concept 7. The closure device according to any one of Inventive Concepts 1-6,
wherein the closure device further includes:
a ferrule, which is coupled to a distal end portion of the suture;
a ferrule-advancing suturing needle, which is removably couplable to the ferrule; and
a ferrule-withdrawing suturing needle,
wherein the suture-positioning support is shaped so as to define a ferrule receptacle, which is configured to removably receive the ferrule,
wherein the elongate support defines, through a longitudinal portion of the elongate support, first and second needle lumens having first and second distal lumen openings, respectively, and
wherein the closure device is configured to:
couple the suture to the suture-positioning support by distally advancing, within the first needle lumen and out of the first distal lumen opening, the ferrule-advancing suturing needle removably coupled to the ferrule, and inserting the ferrule into the ferrule receptacle, and
proximally withdraw the suture from the suture-positioning support by distally advancing, within the second needle lumen and out of the second distal lumen opening, the ferrule-withdrawing suturing needle to the ferrule, coupling the ferrule-withdrawing suturing needle to the ferrule, and proximally withdrawing the ferrule-withdrawing suturing needle while coupled to the ferrule, so as to remove the ferrule from the ferrule receptacle.

Inventive Concept 8. The closure device according to Inventive Concept 7,
wherein a free end of the suture-positioning support near which the ferrule receptacle is disposed is shaped so as to define a wall, and
wherein the ferrule receptacle is shaped so as to define a slit through the wall of the suture-positioning support.

Inventive Concept 9. The closure device according to Inventive Concept 8, wherein the wall is shaped so as to define a recessed portion along one side of the slit, thereby enlarging a longitudinal portion of the slit, which allows the suture to exit the ferrule receptacle during rotation of the suture-positioning support about the distal-support central longitudinal axis.

Inventive Concept 10. The closure device according to Inventive Concept 9, wherein the recessed portion extends along ⅓-⅔ of a length of the slit.

Inventive Concept 11. The closure device according to Inventive Concept 9, wherein the recessed portion defines a flat surface that is angled with respect to a remainder of the wall.

Inventive Concept 12. The closure device according to Inventive Concept 9, wherein the recessed portion defines a curved surface.

Inventive Concept 13. The closure device according to Inventive Concept 9, wherein the wall is shaped so as to define two recessed portions respectively along both sides of the slit.

Inventive Concept 14. The closure device according to Inventive Concept 9, wherein the wall is shaped so as to define exactly one recessed portion along the one side of the slit, and the one side is a trailing side of the slit during rotation of the suture-positioning support about the distal-support central longitudinal axis by the closure device.

Inventive Concept 15. The closure device according to any one of Inventive Concepts 1-6,
wherein the elongate support includes an outer tubular shaft having a distal end,
wherein the suture-positioning support has proximal and distal end portions at opposite ends of the suture-positioning support,
wherein the proximal end portion of the suture-positioning support is coupled to the elongate support, and
wherein the distal end portion of the suture-positioning support is disposed distally to the distal end of the outer tubular shaft.

Inventive Concept 16. The closure device according to Inventive Concept 15, wherein the elongate support further includes an inner shaft nested within the outer tubular shaft, and wherein the suture-positioning support is coupled to the inner shaft of the elongate support.

Inventive Concept 17. The closure device according to any one of Inventive Concepts 1-6,
wherein the suture-positioning support is pivotably coupled to the distal end portion of the elongate support such that the suture-positioning support is rotatable about a pivot axis that is not coaxial with the distal-support central longitudinal axis, and
wherein the closure device is configured to transition the suture-positioning support from the delivery position to the first deployed position by rotating the suture-positioning support about the pivot axis.

Inventive Concept 18. The closure device according to Inventive Concept 17, wherein the closure device is configured to transition the suture-positioning support from the first deployed position back to the delivery position by rotating the suture-positioning support about the pivot axis.

There is further provided, in accordance with an Inventive Concept 19 of the present invention, a closure device for suturing a puncture, the closure device including:
a suture;
a ferrule, coupled to an end portion of the suture;
an elongate support, which includes one or more shafts;
a suture-positioning support, which is (a) coupled to a distal end portion of the elongate support, (b) laterally extendable with respect to the elongate support, and (c) shaped so as to define a ferrule receptacle, which is configured to removably receive the ferrule;
a control handle, which (a) is coupled to a proximal end portion of the elongate support, and (b) includes a wall shaped so as to define a lateral opening through the wall; and
a ferrule-withdrawing suturing needle, which (a) includes a distal end portion that is removably couplable to the ferrule, and (b) is removably disposed entirely within the control handle,
wherein the closure device is configured such that:
distal advancement of the ferrule-withdrawing suturing needle to the ferrule in the ferrule receptacle couples the ferrule-withdrawing suturing needle to the ferrule,
proximal withdrawal of the ferrule-withdrawing suturing needle coupled to the ferrule removes the ferrule from the ferrule receptacle, and
the ferrule-withdrawing suturing needle is removable from the control handle via the lateral opening.

Inventive Concept 20. The closure device according to Inventive Concept 19, wherein the ferrule is coupled to a distal end portion of the suture.

Inventive Concept 21. The closure device according to Inventive Concept 19, wherein the lateral opening is disposed distal to a proximal end portion of the control handle.

Inventive Concept 22. The closure device according to Inventive Concept 19, further including a ferrule-advancing suturing needle, which (a) includes a distal portion that is removably couplable to the ferrule, and (b) is removably disposed entirely within the control handle,
wherein the closure device is configured to direct the ferrule into the ferrule receptacle during distal advancement of the suturing ferrule-advancing needle removably coupled to the ferrule.

Inventive Concept 23. The closure device according to Inventive Concept 19, wherein the closure device is configured such that the control handle prevents removal of the ferrule-withdrawing suturing needle from the control handle until after the proximal withdrawal of the ferrule-withdrawing suturing needle removes the ferrule from the ferrule receptacle.

Inventive Concept 24. The closure device according to any one of Inventive Concepts 19-23, wherein the ferrule-withdrawing suturing needle includes a needle handle, which is moveable separately from the needle user control, and which coupled to a proximal end of the ferrule-withdrawing suturing needle, wherein the ferrule-withdrawing suturing needle, including the needle handle and the distal end portion of the ferrule-withdrawing suturing needle, is removably disposed entirely within the control handle.

Inventive Concept 25. The closure device according to Inventive Concept 24, wherein the control handle is configured to at least partially eject the needle handle from the lateral opening after the proximal withdrawal of the ferrule-withdrawing suturing needle removes the ferrule from the ferrule receptacle.

Inventive Concept 26. The closure device according to Inventive Concept 25, wherein the closure device is configured such that the control handle prevents removal of the ferrule-withdrawing suturing needle from the control handle until after the proximal withdrawal of the ferrule-withdrawing suturing needle removes the ferrule from the ferrule receptacle.

Inventive Concept 27. The closure device according to any one of Inventive Concepts 19-23, wherein the control handle includes a needle user control that is permanently coupled to the control handle, and wherein the control handle is configured such that:
actuation of the needle user control causes the distal advancement of the ferrule-withdrawing suturing needle to the ferrule in the ferrule receptacle, and
subsequent actuation of the needle user control causes the proximal withdrawal of the ferrule-withdrawing suturing needle coupled to the ferrule.

Inventive Concept 28. The closure device according to Inventive Concept 27, wherein the ferrule-withdrawing suturing needle includes a needle handle coupled to a proximal end of the ferrule-withdrawing suturing needle, wherein the ferrule-withdrawing suturing needle, including the needle handle and the distal end portion of the ferrule-withdrawing suturing needle, is removably disposed entirely within the control handle.

Inventive Concept 29. The closure device according to Inventive Concept 27, wherein the subsequent actuation of the needle user control is caused by releasing the user control.

Inventive Concept 30. The closure device according to Inventive Concept 27, wherein the needle user control includes a support-and-needle user control that is permanently coupled to the control handle, and wherein the control handle is configured such that:
the actuation of the support-and-needle user control causes (a) the distal advancement of the ferrule-withdrawing suturing needle to the ferrule in the ferrule receptacle, and (b) lateral extension of the suture-positioning support, with respect to the elongate support, from a delivery position to a deployed position, and
the subsequent actuation of the support-and-needle user control causes (a) the proximal withdrawal of the ferrule-withdrawing suturing needle coupled to the ferrule, and (b) transitioning of the suture-positioning support from the deployed position back to the delivery position.

There is still further provided, in accordance with an Inventive Concept 31 of the present invention, apparatus including:
a suture;
a ferrule-withdrawing suturing needle;
a ferrule, which (a) is coupled to a distal end portion of the suture, and (b) is shaped so as to define (i) a ferrule lumen and (ii) one or more tabs, which are biased to protrude radially inward within the ferrule lumen and to engage the ferrule-withdrawing suturing needle upon insertion of the ferrule-withdrawing suturing needle into an end opening of the ferrule lumen, thereby inhibiting withdrawal of the ferrule-withdrawing suturing needle from the ferrule lumen; and
a ferrule-advancing suturing needle, which includes a distal end portion that is shaped and sized so as, upon insertion of the distal end portion into the ferrule lumen, to provide space within the ferrule lumen for the one or more tabs, and to contact an internal surface of the ferrule lumen so as to grasp the ferrule.

Inventive Concept 32. The apparatus according to Inventive Concept 31, wherein the suture is disposed partially within the ferrule lumen, and wherein the ferrule-advancing suturing needle is shaped and sized so as, upon the insertion of the distal end portion into the ferrule lumen, to provide space within the ferrule lumen for the suture and the one or more tabs, and to contact an internal surface of the ferrule lumen so as to grasp the ferrule.

Inventive Concept 33. The apparatus according to any one of Inventive Concepts 31-32, wherein the distal end portion of the ferrule-advancing suturing needle is shaped so as to define two or more elongate indentations, which are indented radially inwardly with respect to a geometric cylinder defined by radially-outmost portions of the distal end portion.

Inventive Concept 34. The apparatus according to Inventive Concept 33, wherein the distal end portion of the ferrule-advancing suturing needle is shaped so as to define two or more flat sides that define the two or more elongate indentations, respectively.

Inventive Concept 35. The apparatus according to Inventive Concept 34, wherein the distal end portion of the ferrule-advancing suturing needle is shaped so as to define three flat sides.

There is additionally provided, in accordance with an Inventive Concept 36 of the present invention, a closure device for suturing a puncture, the closure device including:
a suture;
an elongate support, which includes one or more shafts;
a ferrule, which is coupled to a distal end portion of the suture;
a suturing needle, which is removably couplable to the ferrule;
a suture-positioning support, which is (a) coupled to a distal end portion of the elongate support, (b) laterally extendable with respect to the distal end portion of the elongate support, and (c) shaped so as to define a ferrule receptacle, which is configured to removably receive the ferrule, such that the suture-positioning support is configured to removably receive the suture; and
a control handle, which (i) includes a support-and-needle user control, and (ii) is configured such that actuation of the support-and-needle user control causes:
lateral extension of the suture-positioning support, with respect to a distal end portion of the elongate support, from (a) a delivery position, in which a suture-positioning support axis of the suture-positioning support forms a first angle of less than 45 degrees with a distal-support central longitudinal axis of the distal end portion of the elongate support, to (b) a deployed position in which the suture-positioning support laterally extends from the distal end portion of the elongate support, and
distal advancement of the suturing needle, while the suturing needle is removably coupled to the ferrule, such that the closure device directs the ferrule into the ferrule receptacle and the ferrule receptacle removably receives the ferrule while the suture-positioning support is in the deployed position.

Inventive Concept 37. The closure device according to Inventive Concept 36, wherein the control handle is configured such that the actuation of the support-and-needle user control causes at least a portion of the distal advancement of the suturing needle to occur as the suture-positioning support is extended laterally.

Inventive Concept 38. The closure device according to Inventive Concept 37, wherein the control handle is configured such that the actuation of the support-and-needle user control causes the suture-positioning support to begin extending laterally before the suturing needle begins advancing distally.

Inventive Concept 39. The closure device according to Inventive Concept 36, wherein the control handle is configured such that the actuation of the support-and-needle user control non-electrically causes the distal advancement of the suturing needle and the lateral extension of the suture-positioning support.

Inventive Concept 40. The closure device according to Inventive Concept 36, wherein the support-and-needle user control is configured to be actuated by movement of the user control in a proximal-to-distal direction.

Inventive Concept 41. The closure device according to any one of Inventive Concepts 36-40,
wherein the suture-positioning support is pivotably coupled to the distal end portion of the elongate support such that suture-positioning support is rotatable about a pivot axis to transition between the delivery position and the deployed position, the pivot axis not coaxial with the distal-support central longitudinal axis,
wherein the control handle is shaped so as to define an internal proximal stopper surface, which faces at least partially proximally, and
wherein the control handle includes:
an extension-control-shaft that is coupled to a site on the suture-positioning support away from the pivot axis;
a proximal slider, which is axially moveable within the control handle;
a distal slider, which is (a) axially moveable within control handle with respect to the proximal stopper surface, so that the proximal stopper surface limits distal advancement of the distal slider, and (b) axially fixed to the extension-control-shaft, such that the distal advancement of the distal slider distally advances the extension-control-shaft, thereby causing the lateral extension of the suture-positioning support to the deployed position;
a proximal spring, which is disposed so as to resist movement of the proximal slider and the distal slider toward each other; and
a distal spring, which is disposed so as to resist the distal advancement of the distal slider toward the proximal stopper surface.

Inventive Concept 42. The closure device according to Inventive Concept 41,
wherein the proximal slider is shaped so as to define one or more slider engagement surfaces,
wherein the control handle includes a needle-control shaft, which includes a shaft engagement surface,
wherein the support-and-needle user control is configured to engage the needle-control shaft, and
wherein the actuation of the support-and-needle user control, when the needle-control shaft is engaged by the support-and-needle user control, distally advances the needle-control shaft, such that the shaft engagement surface of the needle-control shaft engages one or more of the one or more slider engagement surfaces of the proximal slider, and distally pushes the proximal slider.

Inventive Concept 43. The closure device according to any one of Inventive Concepts 36-40, wherein the control handle is configured such that:
the actuation of the support-and-needle user control to cause the lateral extension and the distal advancement of the suturing needle is initial actuation of the support-and-needle user control, and
subsequent actuation of the support-and-needle user control, after the initial actuation of the support-and-needle user control, causes:
proximal withdrawal of the suturing needle, a portion of which proximal withdrawal proximally withdraws the suturing needle from the ferrule while leaving the ferrule within the ferrule receptacle, and
transitioning of the suture-positioning support from the deployed position back to the delivery position.

Inventive Concept 44. The closure device according to Inventive Concept 43, wherein the control handle is configured such that the subsequent actuation of the support-and-needle user control causes at least a portion of the transitioning of the suture-positioning support from the deployed position back to the delivery position to occur as the suturing needle is proximally withdrawn.

Inventive Concept 45. The closure device according to Inventive Concept 43,
wherein the deployed position is a first deployed position in which the suture-positioning support laterally extends from the distal end portion of the elongate support in a first direction,
wherein the suturing needle is a ferrule-advancing suturing needle,
wherein the closure device further includes a ferrule-withdrawing suturing needle,
wherein the control handle is configured such that rotation of the support-and-needle user control rotates the suture-positioning support about the distal-support central longitudinal axis, and
wherein the control handle is configured such that second subsequent actuation of the support-and-needle user control, after the rotation of the support-and-needle user control, causes:
lateral extension of the suture-positioning support, with respect to the distal end portion of the elongate support, from (a) the delivery position to (b) a second deployed position in which the suture-positioning support laterally extends from the distal end portion of the elongate support in a second direction different from the first direction, and
distal advancement of the ferrule-withdrawing suturing needle, such that the closure device directs the ferrule-withdrawing suturing needle to the ferrule and couples the ferrule-withdrawing suturing needle to the ferrule while the suture-positioning support is in the second deployed position.

Inventive Concept 46. The closure device according to Inventive Concept 43, wherein the subsequent actuation of the needle user control is caused by releasing the user control.

Inventive Concept 47. The closure device according to any one of Inventive Concepts 36-46,
wherein the suture is a first suture, and wherein the closure device further includes a second suture,
wherein the ferrule is a first ferrule, and wherein the closure device further includes a second ferrule, which is coupled to a distal end portion of the second suture, and
wherein the ferrule receptacle of the suture-positioning support is configured to removably receive the second ferrule.

Inventive Concept 48. The closure device according to any one of Inventive Concepts 36-47, wherein the control handle is configured such that the actuation of the support-and-needle user control causes the suture-positioning support to finish extending laterally before the suturing needle reaches the suture-positioning support.

Inventive Concept 49. The closure device according to any one of Inventive Concepts 36-48, wherein the control handle is configured such that the actuation of the support-and-needle user control causes the suture-positioning support to finish extending laterally before the suturing needle begins advancing distally.

There is yet additionally provided, in accordance with an Inventive Concept 50 of the present invention, a closure device for suturing a puncture, the closure device including:
a suture;
an elongate support, which includes an outer tubular shaft having a distal end,
a suture-positioning support, which is (a) coupled to a distal end portion of the elongate support, (b) laterally extendable with respect to the elongate support, and (c) configured to removably receive the suture;
an elongate dilator; and
a sheath that covers: the distal end of the outer tubular shaft, the suture-positioning support, and a proximal end portion of the dilator that includes the proximal end of the dilator,
wherein the sheath is proximally withdrawable along the outer tubular shaft so as to expose the suture-positioning support and the proximal end portion of the dilator.

Inventive Concept 51. The closure device according to Inventive Concept 50,
further including an elongate flexible dilator connector, which couples the proximal end of the dilator to the distal end portion of the elongate support so as to allow the distal end portion of the elongate support to move with respect to the proximal end of the dilator,
wherein the sheath covers: the distal end of the outer tubular shaft, the suture-positioning support, the proximal end portion of the dilator, and the dilator connector, and
wherein the sheath is proximally withdrawable along the outer tubular shaft so as to expose the suture-positioning support, the proximal end portion of the dilator, and the dilator connector.

Inventive Concept 52. The closure device according to Inventive Concept 50,
wherein the suture is a first suture, and wherein the closure device further includes a second suture, and
wherein the suture-positioning support is configured to removably receive the second suture.

Inventive Concept 53. The closure device according to Inventive Concept 50, wherein the dilator is shaped so as to define an atraumatic proximal tip.

Inventive Concept 54. The closure device according to Inventive Concept 53, wherein the proximal end portion of the dilator is shaped so as to define an indentation at least partially around the dilator longitudinally between the atraumatic proximal tip and the remaining more distal portion of the dilator.

Inventive Concept 55. The closure device according to any one of Inventive Concepts 50-54,
wherein the closure device further includes a control handle, which is coupled to a proximal end portion of the elongate support, and which includes a sheath-control user control, and
wherein the closure device is configured such that actuation of the sheath-control user control proximally withdraws sheath along the outer tubular shaft.

Inventive Concept 56. The closure device according to Inventive Concept 55,
wherein the control handle (i) includes a support-and-needle user control, and (ii) is configured such that actuation of the support-and-needle user control causes lateral extension of the suture-positioning support, with respect to a distal end portion of the elongate support, from (a) a delivery position, in which a suture-positioning support axis of the suture-positioning support forms a first angle of less than 45 degrees with a distal-support central longitudinal axis of the distal end portion of the elongate support, to (b) a deployed position in which the suture-positioning support laterally extends from the distal end portion of the elongate support, and
wherein the control handle is configured to lock the support-and-needle user control, thereby preventing the actuation thereof, until the actuation of the sheath-control user control.

Inventive Concept 57. The closure device according to Inventive Concept 56, wherein the control handle is configured to lock the support-and-needle user control by preventing rotation of the support-and-needle user control.

Inventive Concept 58. The closure device according to Inventive Concept 57, wherein the control handle is configured to lock the support-and-needle user control by preventing the rotation of the support-and-needle user control while the support-and-needle user control is in a non-operational rotational position in which the support-and-needle user control cannot be actuated to cause the lateral extension of the suture-positioning support.

Inventive Concept 59. The closure device according to Inventive Concept 58,
wherein the control handle includes a key-bearing shaft, and a proximal end portion of the key-bearing shaft defines a key,
wherein the support-and-needle user control is shaped so as to define a keyway,
wherein the sheath-control user control is in a non-actuated state until the actuation of the sheath-control user control, and
wherein when the sheath-control user control is in the non-actuated state, the key is within the keyway, thereby preventing rotation of the support-and-needle user control and thus locking the support-and-needle user control.

Inventive Concept 60. The closure device according to any one of Inventive Concepts 50-54,
wherein the closure device further includes a control handle, which is coupled to a proximal end portion of the elongate support, and
wherein the closure device is configured such that when the sheath covers the distal end of the outer tubular shaft, the suture-positioning support, and the proximal end portion of the dilator:
the sheath, the outer tubular shaft of the elongate support, and a distal portion of the control handle are configured to provide a blood flow path between outside the sheath and a lateral opening defined by the distal portion of the control handle.

Inventive Concept 61. The closure device according to Inventive Concept 60, wherein the blood flow path is defined partially by an interior of the sheath.

Inventive Concept 62. The closure device according to Inventive Concept 61, wherein the sheath is shaped so as to define one or more openings between outside the sheath and the interior of the sheath.

Inventive Concept 63. The closure device according to Inventive Concept 61, wherein the elongate support further includes an annular seal that surrounds the outer tubular shaft and contacts an inner surface of the sheath when the sheath is in its distally advanced position, thereby preventing proximal blood flow from the interior of the sheath into a space between an outer surface of the outer tubular shaft and the inner surface of the sheath, so as to direct the proximal blood flow along the blood flow path within the outer tubular shaft.

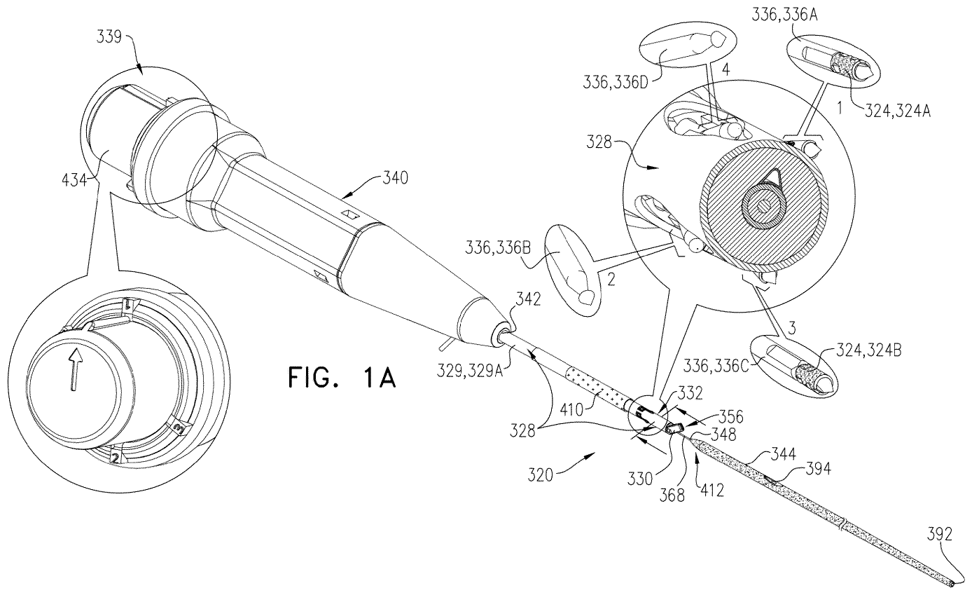

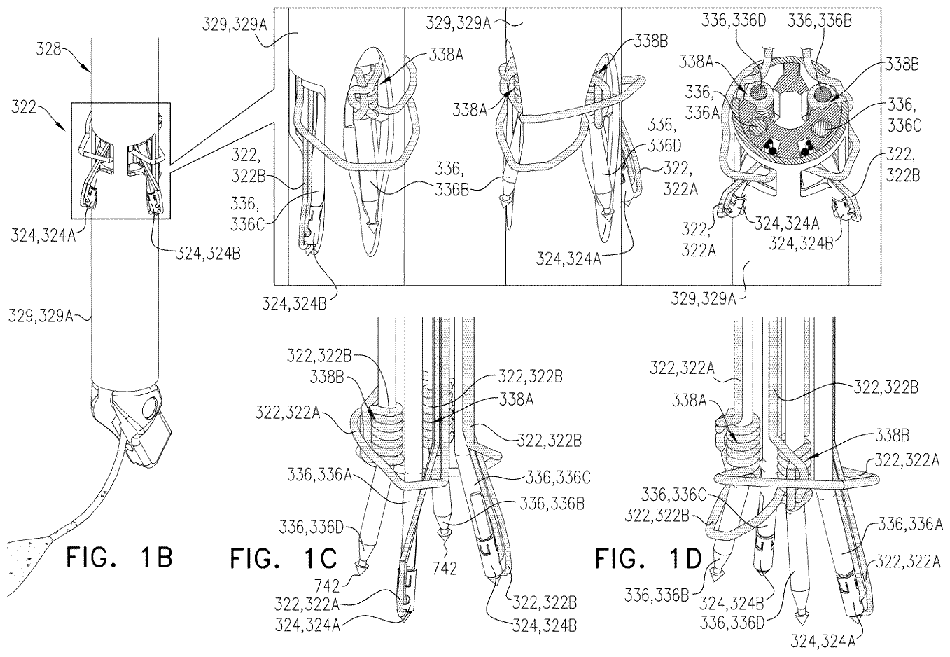

Reference is made to FIG. 1A, which is a schematic illustration of a closure device 320 for suturing a puncture at an access site with at least one suture 322, in accordance with an application of the present invention. For the sake of clarity suture 322 is not shown in FIG. 1A, although the suture is present in practice; the suture is shown in many of the other figures described hereinbelow.

Reference is further made to FIG. 1B, which is a schematic illustration of a distal end portion 332 of an elongate support 328 of closure device 320, in accordance with an application of the present invention.

Reference is still further made to FIGS. 1C-D, which are schematic illustrations of distal portions of suturing needles 336 and sutures 322 of closure device 320, from two views, in accordance with an application of the present invention.

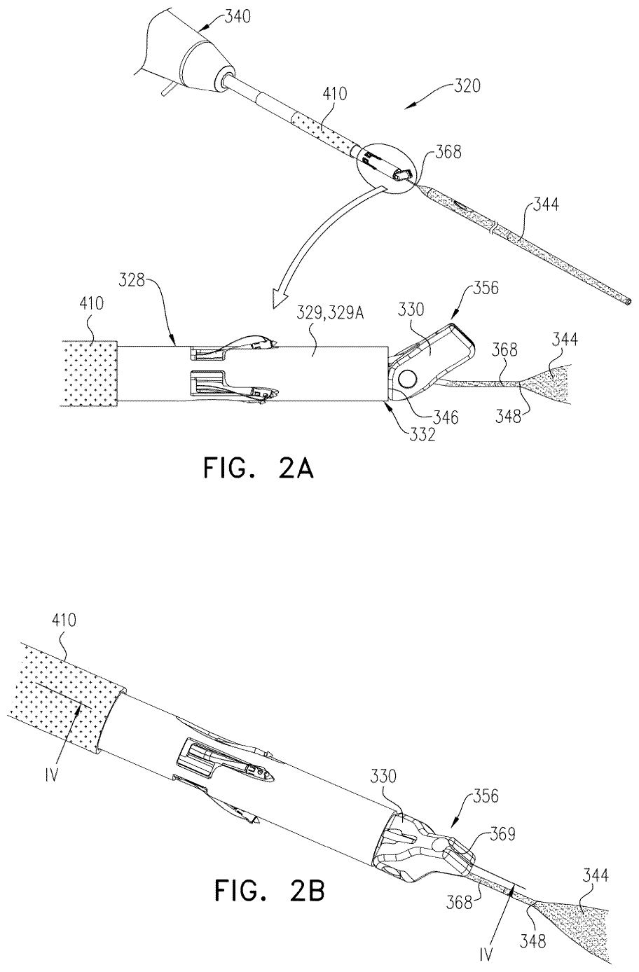

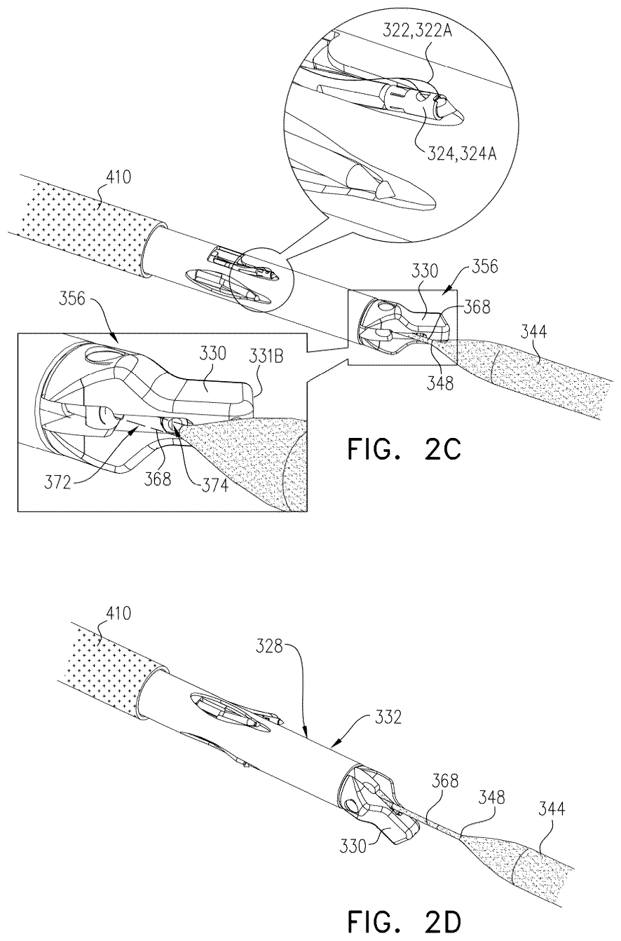

Reference is still further made to FIGS. 2A-D, which are several schematic views of a portion of closure device 320, in accordance with an application of the present invention.

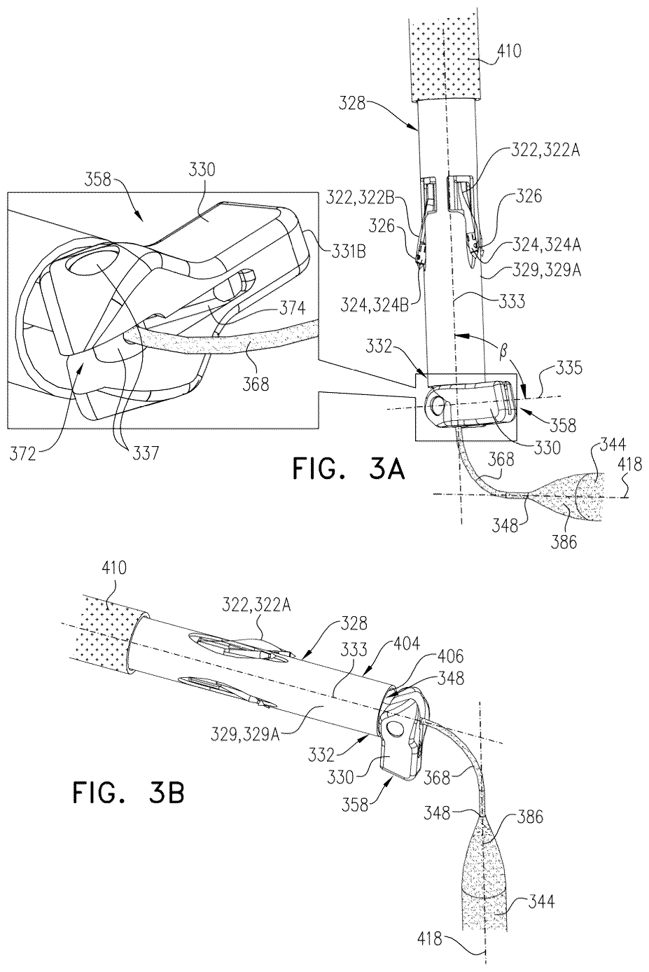

Reference is additionally made to FIGS. 3A-B, which are schematic illustrations of a suture-positioning support 330 of closure device 320 laterally extended from distal end portion 332 of elongate support 328, in accordance with an application of the present invention.

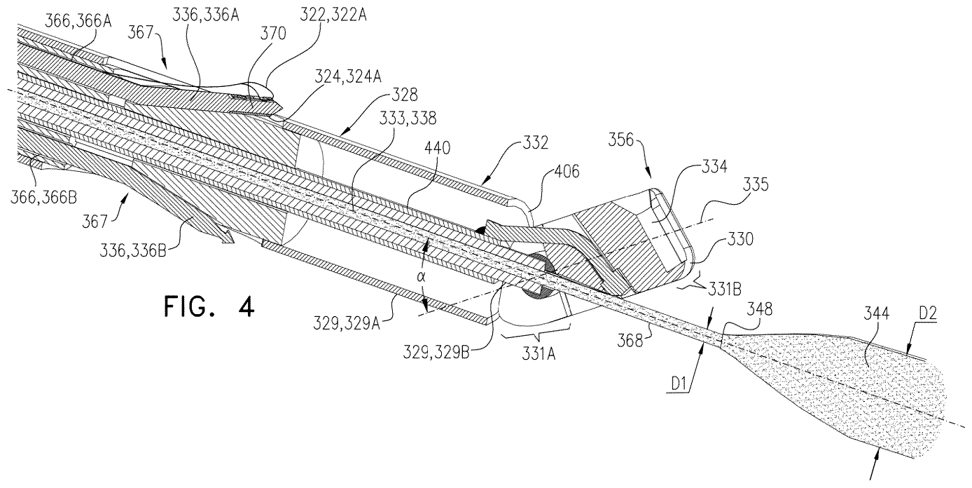

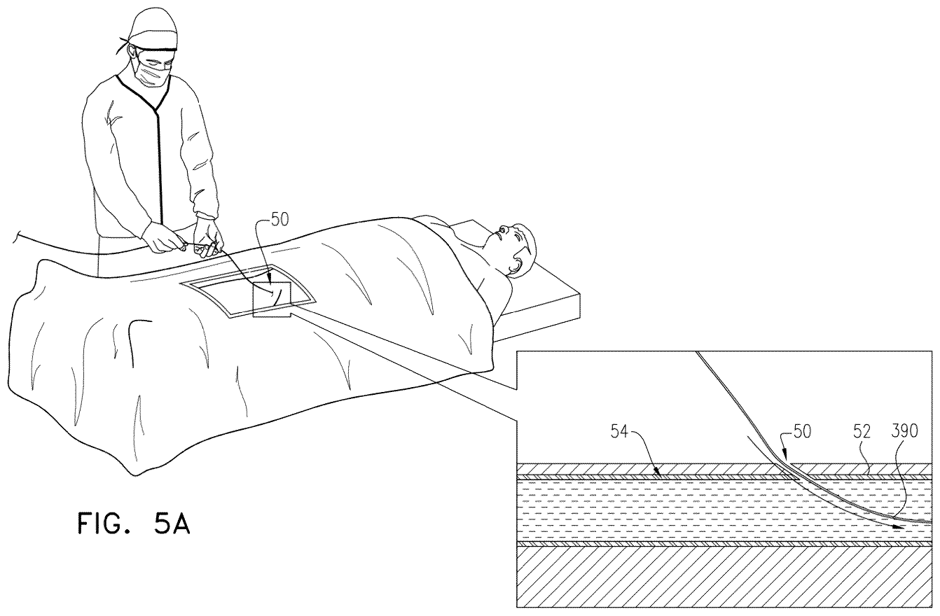

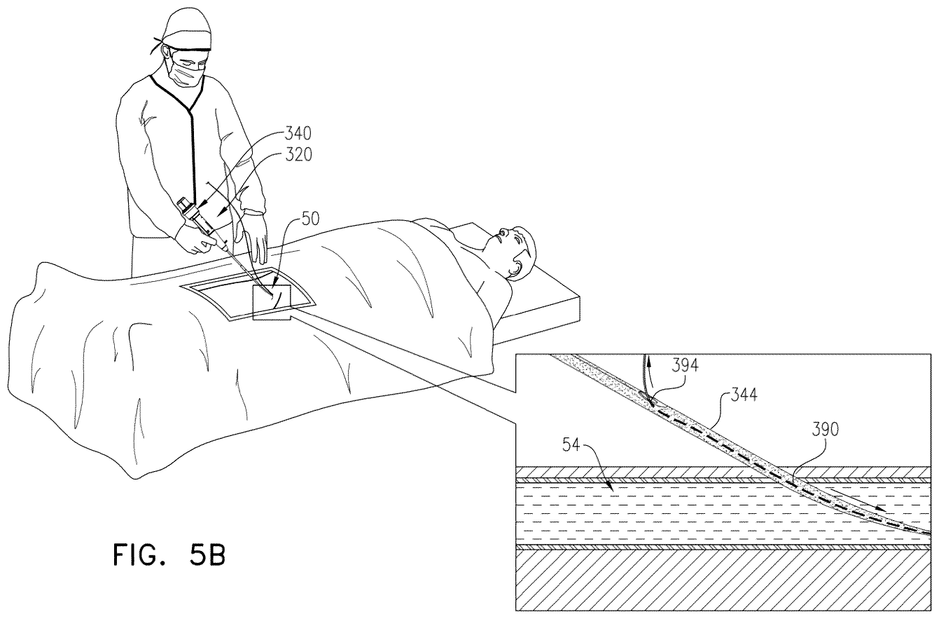

Reference is also made to FIG. 4, which is a schematic cross-sectional view of a portion of closure device 320, in accordance with an application of the present invention.

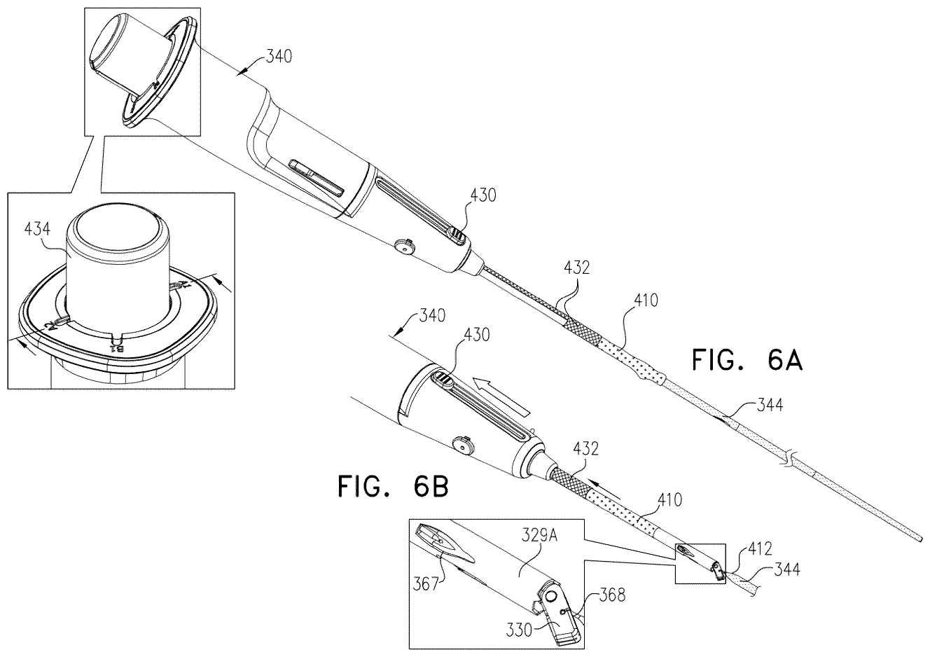

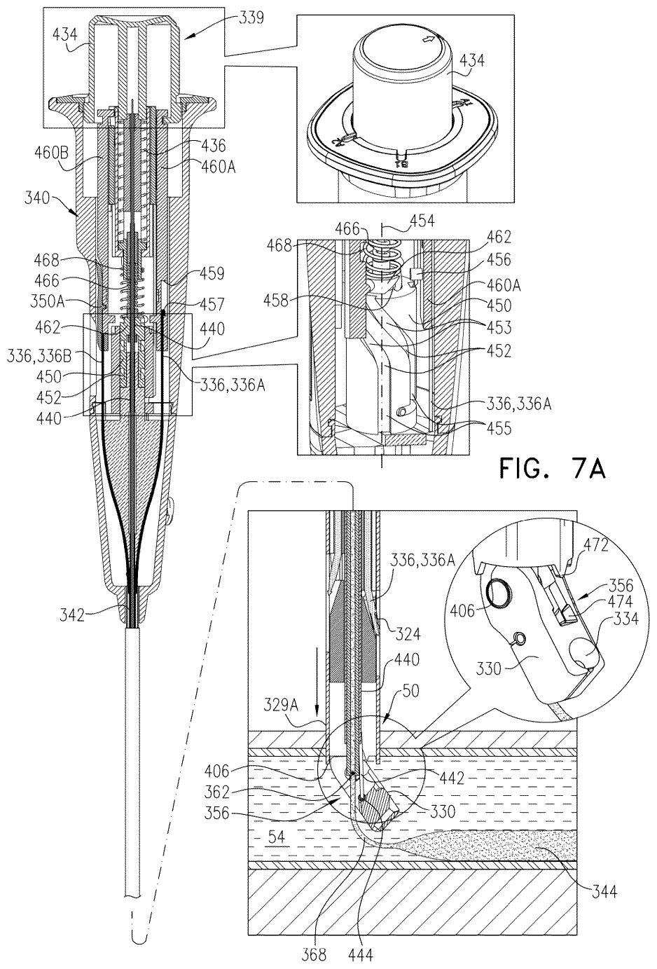

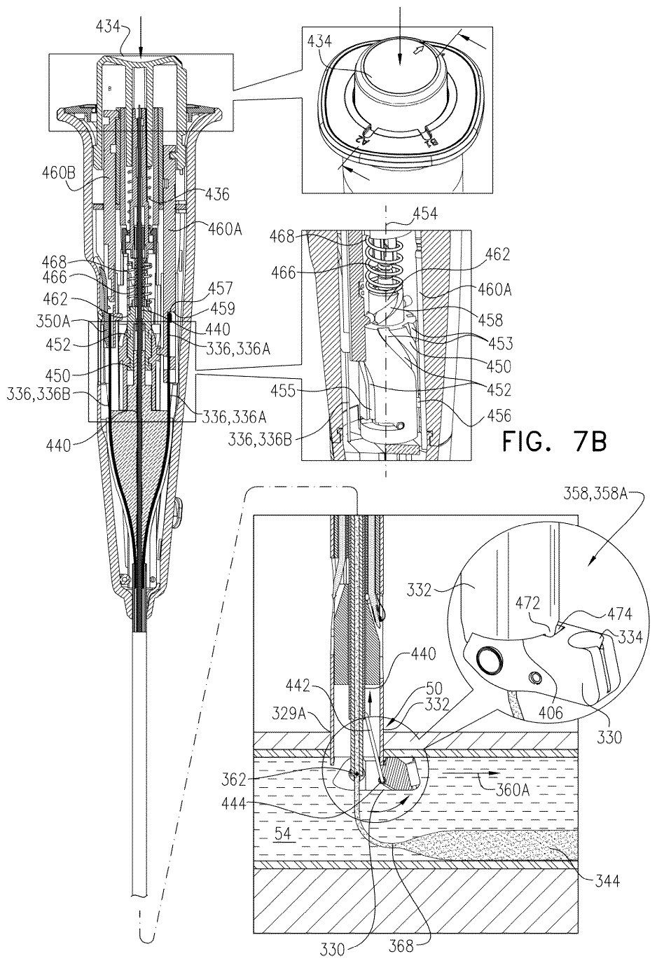

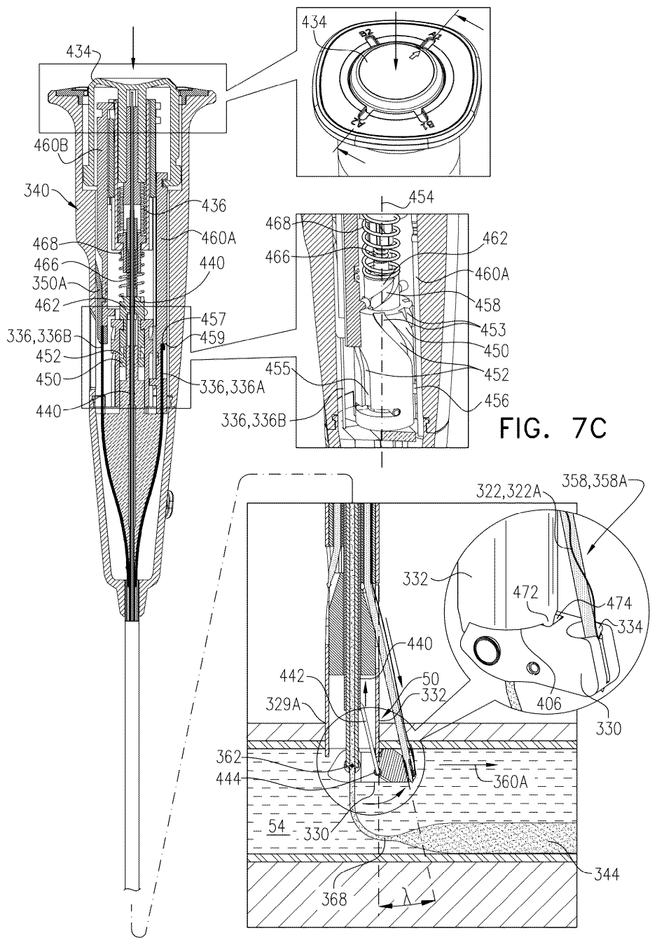

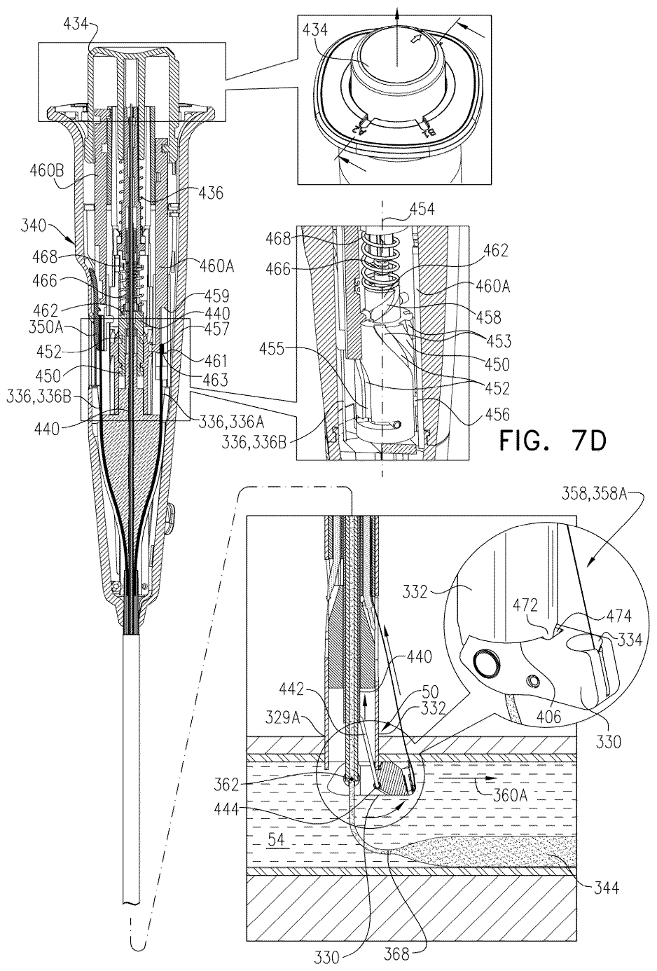

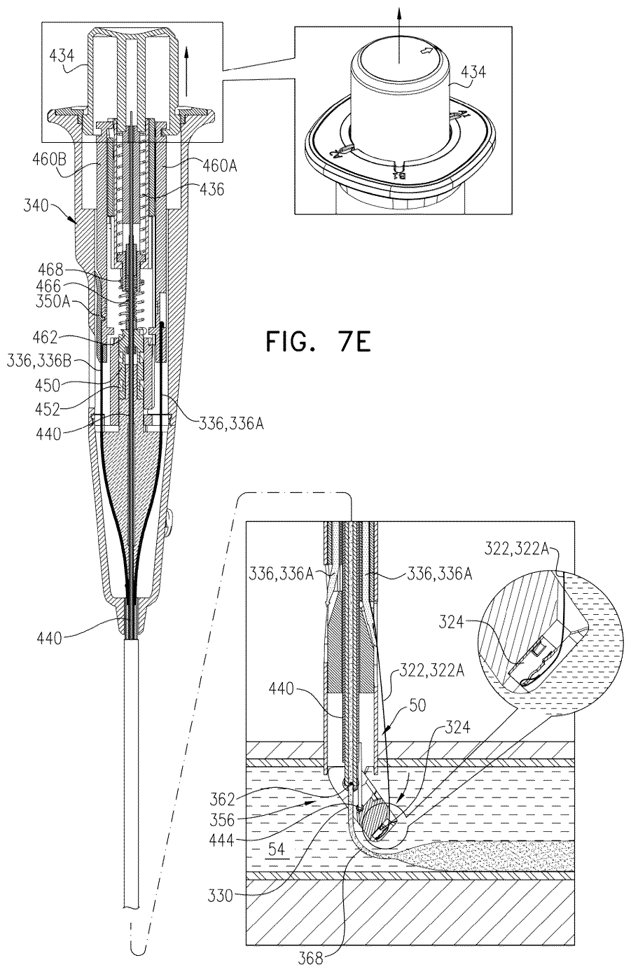

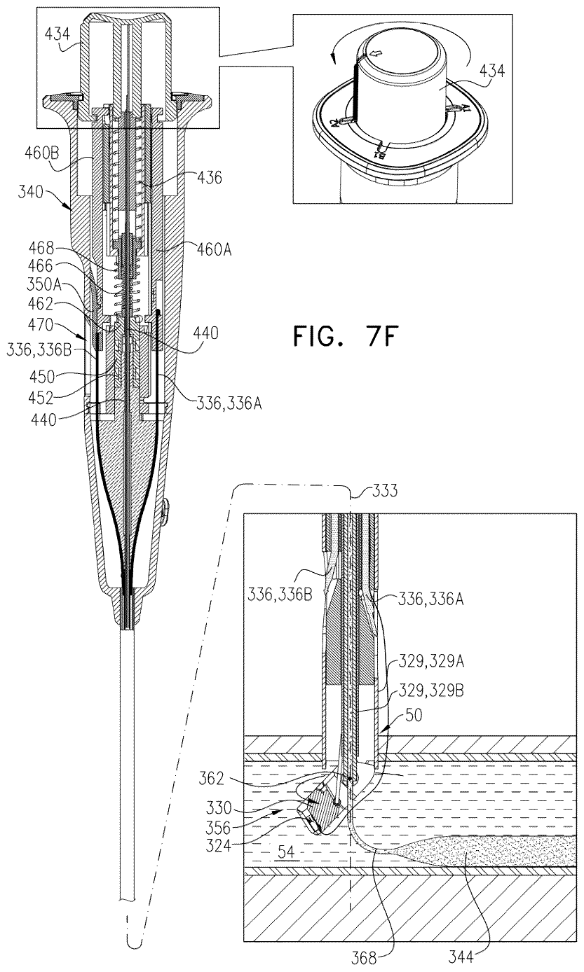

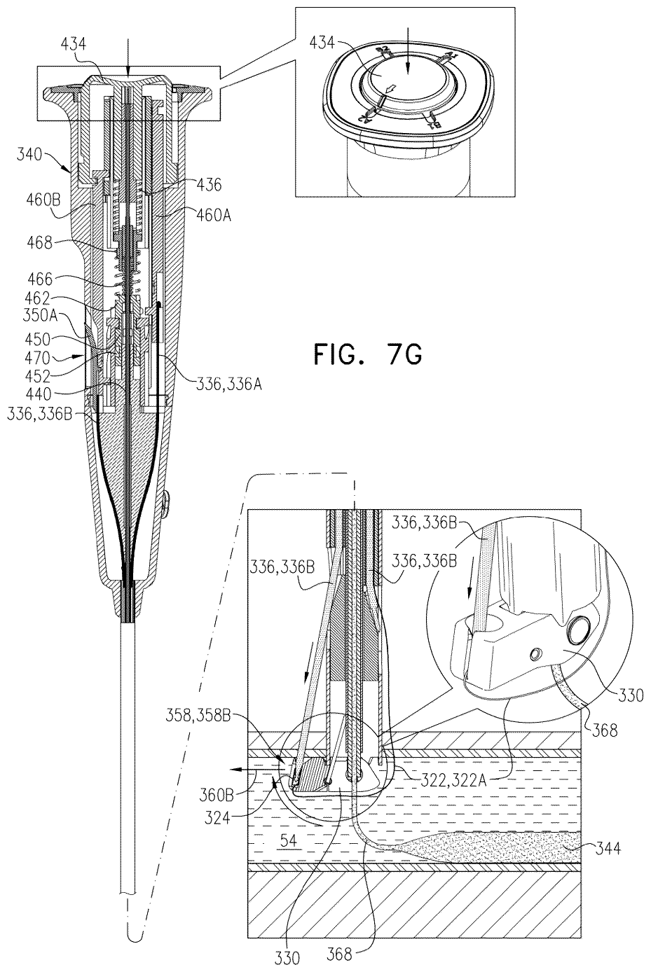

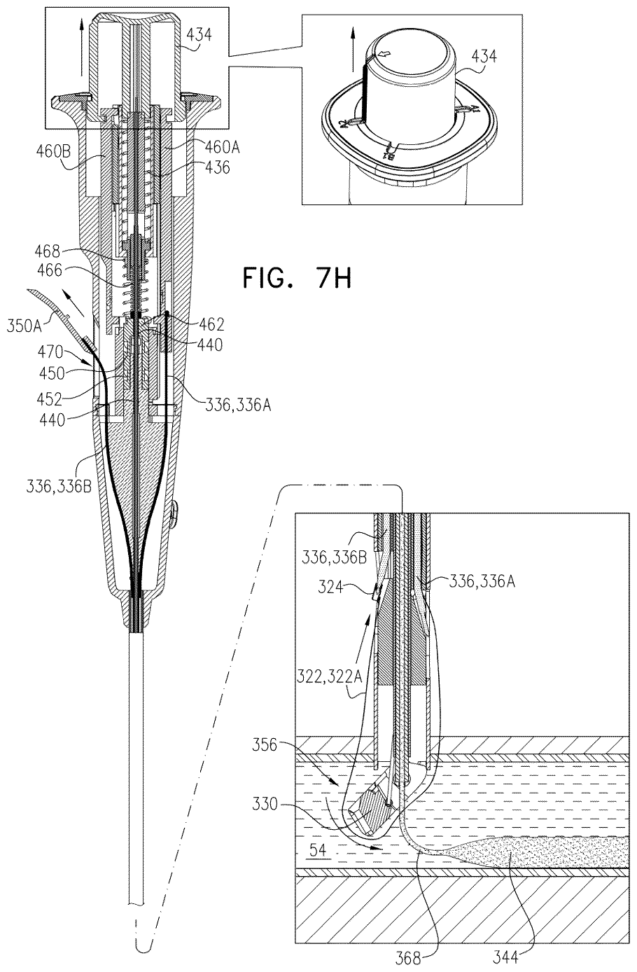

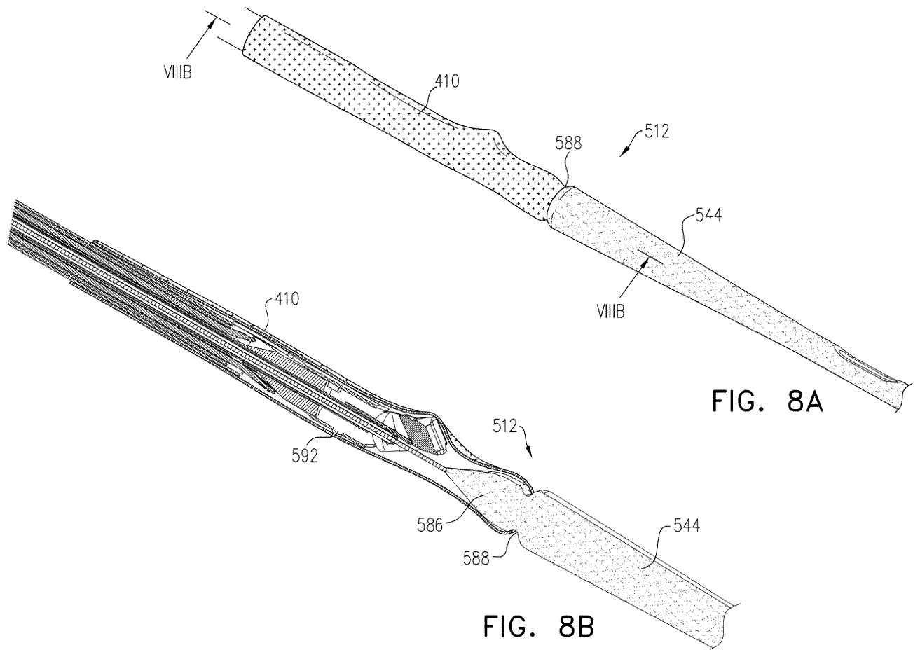

Optionally, closure device 320 may implement, mutatis mutandis, any of the features of closure device 20, described in US Patent Application Publication 2024/0081800 to Iamberger et al. (hereinbelow, the '1800 publication, which is incorporated herein by reference) with reference to FIGS. 1-5J; closure device 120, described in the '1800 publication with reference to FIGS. 6 and 7A-L; and/or closure device 220, described in the '1800 publication with reference to FIGS. 8A-H.

Closure device 320 is used for suturing a puncture at an access site through a wall of a hollow anatomical structure, typically a blood vessel (in which case the access site is a vascular access site), or another body cavity, e.g., an abdominal cavity.

Closure device 320 comprises:
elongate support 328, which typically comprises one or more shafts 329;
suture-positioning support 330, which is (a) coupled to distal end portion 332 of elongate support 328, (b) laterally extendable with respect to distal end portion 332 of elongate support 328, and (c) configured to removably receive the suture 322; and
an elongate dilator 344, which is configured to be inserted through the puncture, and which has a proximal end 348 that is coupled to distal end portion 332 of elongate support 328; dilator 344 typically facilitates insertion and alignment of suture-positioning support 330 in a blood vessel.

Typically, closure device 320 further comprises a control handle 340, coupled to a proximal end portion 342 of elongate support 328, such as shown in FIG. 1A. Control handle 340 comprises one or more user controls, such as levers and/or buttons, that allow an operator to control the positions and states of one or more elements of closure device 320, for example as described hereinbelow with reference to FIGS. 6A-B and 7A-H.

For some applications, closure device 320 is configured to allow movement of distal end portion 332 of elongate support 328 with respect to proximal end 348 of dilator 344:
with at least two degrees of freedom, and/or
with at least one translational degree of freedom.

The term “degrees of freedom” is used in the conventional sense in the present application, including in the claims and the Inventive Concepts, namely the six mechanical degrees of freedom of movement of an object in space, consisting of (a) three translational degrees of freedom in three perpendicular axes (forward/backward (surge), up/down (heave), left/right (sway)), resulting in change in position, and (b) three rotational degrees of freedom about three perpendicular axes (yaw (normal axis), pitch (transverse axis), and roll (longitudinal axis), resulting in changes in orientation.

For some applications, closure device 320 is configured to allow the movement:
without requiring distal end portion 332 of elongate support 328 to directly or indirectly apply a force to proximal end 348 of dilator 344,
without requiring bending of dilator 344, and/or
without requiring deformation of dilator 344.

For some applications, the above-mentioned at least two degrees of freedom include at least one rotational degree of freedom, e.g., at least two rotational degrees of freedom, such as three rotational degrees of freedom.

Alternatively or additionally, for some applications, the above-mentioned at least two degrees of freedom include at least one translational degree of freedom. Optionally, the at least one translational degree of freedom includes a translational degree of freedom along a distal-support central longitudinal axis 333 of distal end portion 332 of elongate support 328 (labeled in FIGS. 3A-B). For some applications, the at least two degrees of freedom include at least two translational degrees of freedom, such as three translational degrees of freedom.

For some applications, the above-mentioned at least one translational degree of freedom includes at least two translational degrees of freedom, such as three translational degrees of freedom.

For some applications, closure device 320 is configured to allow the movement of distal end portion 332 of elongate support 328 with respect to proximal end 348 of dilator 344 with at least four degrees of freedom, such as with six degrees of freedom.

Typically, closure device 320 is configured to allow the above-described movement without causing permanent deformation of any elements of closure device 320. Thus, the movement is reversable, such that distal end portion 332 of elongate support 328 can return to its prior position with respect to proximal end 348 of dilator 344.

Typically, dilator 344 has a length of at least 5 cm (e.g., at least 15 cm), no more than 30 cm (e.g., no more than 25 cm), and/or 5-30 cm (e.g., 15-25 cm), and/or a greatest diameter of at least 2 mm, no more than 6 mm, and/or 2-6 mm.

For some applications, dilator 344 comprises a flexible polymer, e.g., having a hardness of between 30-70 Shore D, optionally including a hydrophilic coating for minimal friction during insertion.

For some applications, dilator 344 is shaped so as to define an atraumatic proximal tip 386 (labeled in FIGS. 3A-B and 5C). Optionally, atraumatic proximal tip 386 is tapered.

For some applications, elongate support 328 comprises an outer tubular shaft 329A having a distal end portion 404 including a distal end 406 of outer tubular shaft 329A (labeled in FIG. 3B).

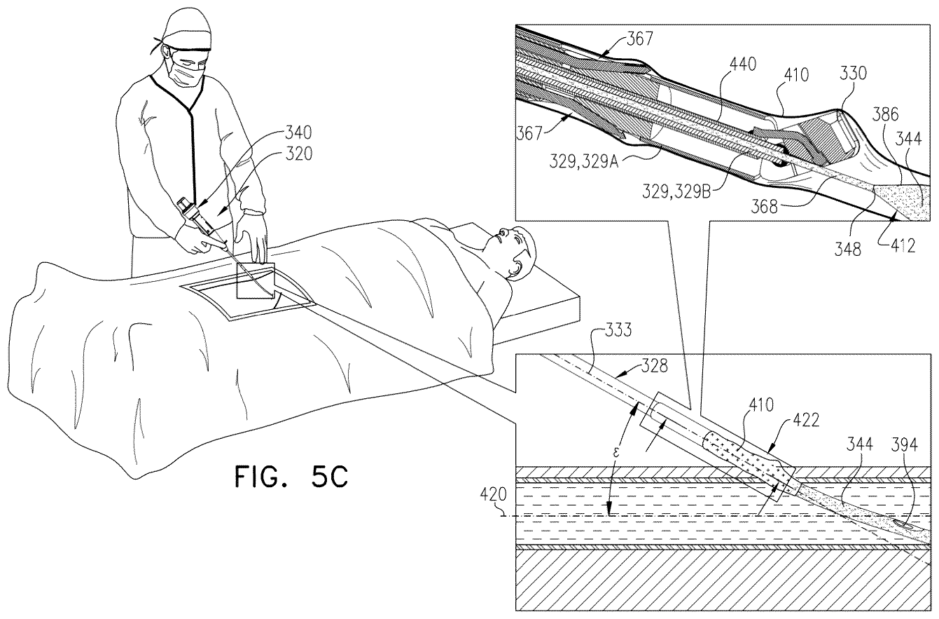

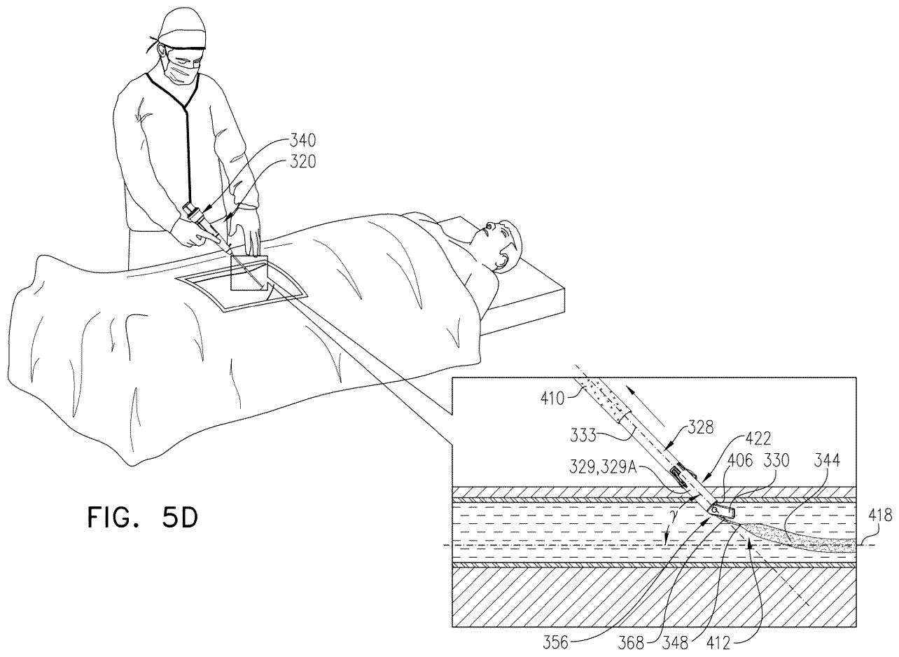

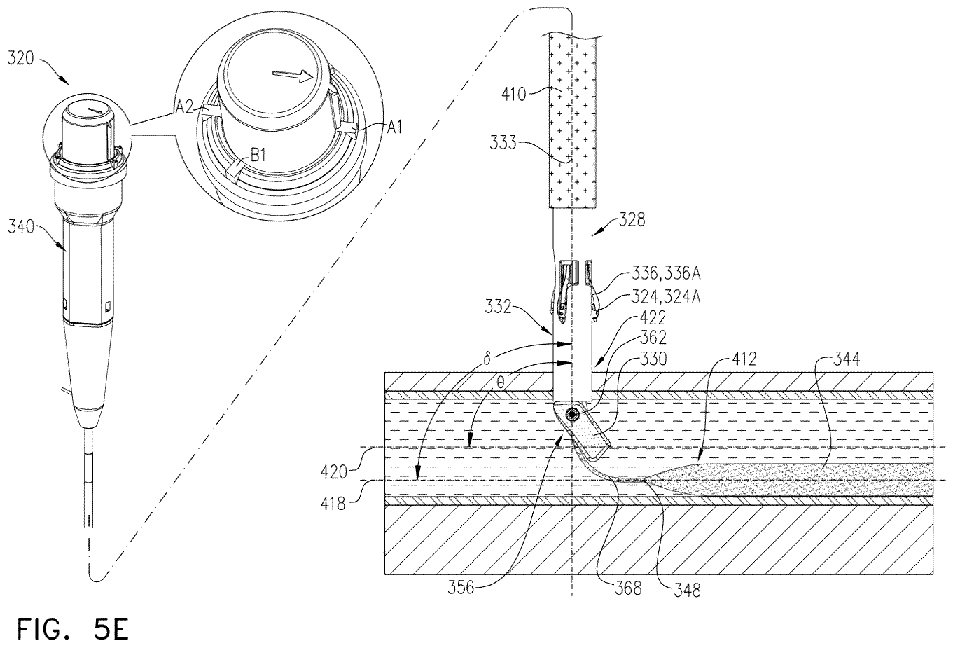

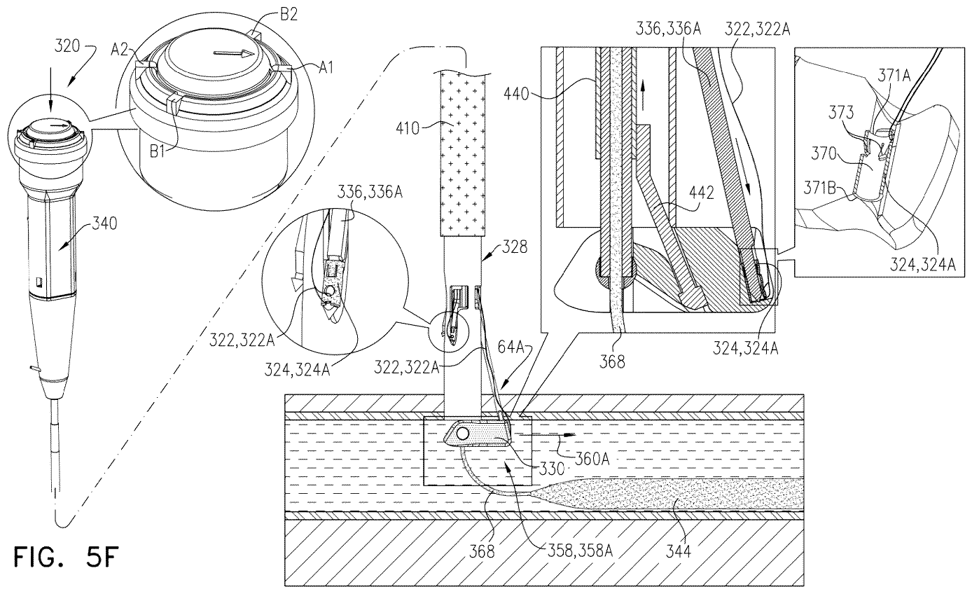

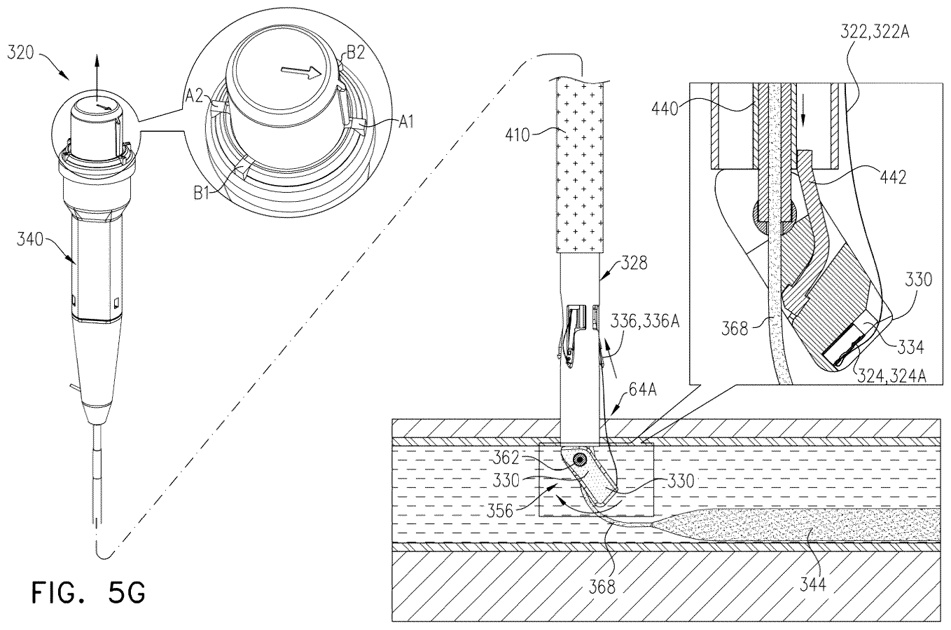

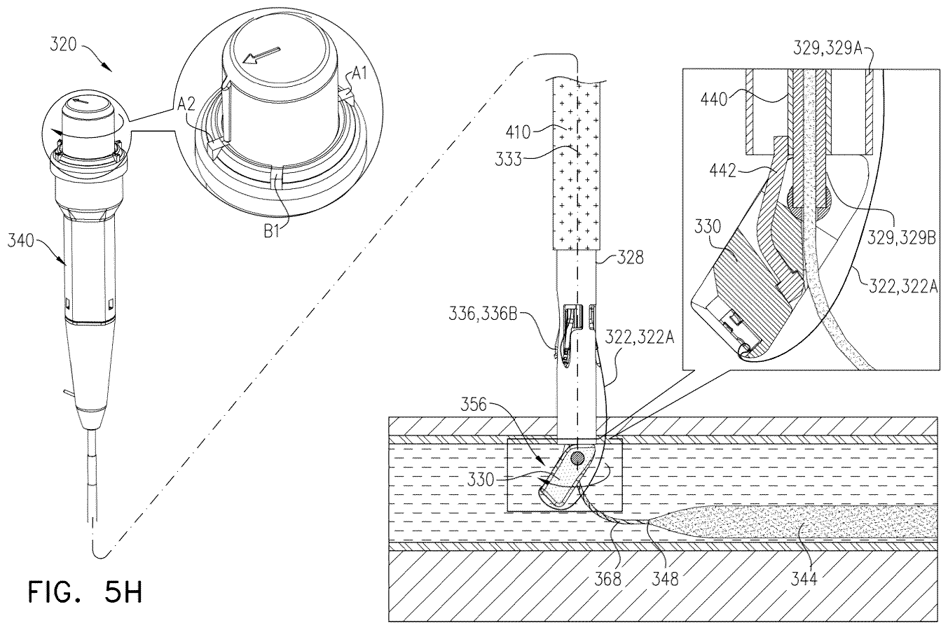

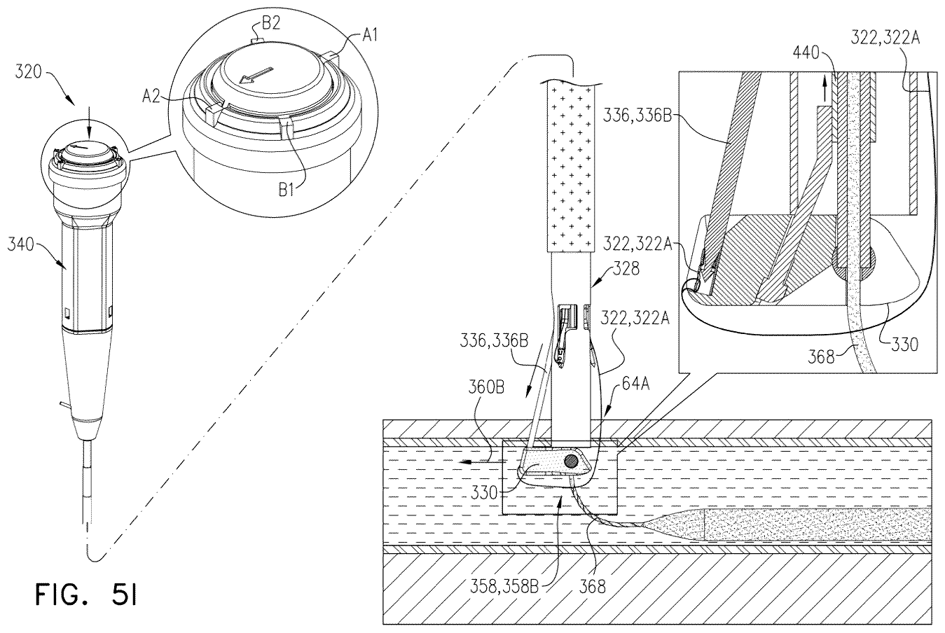

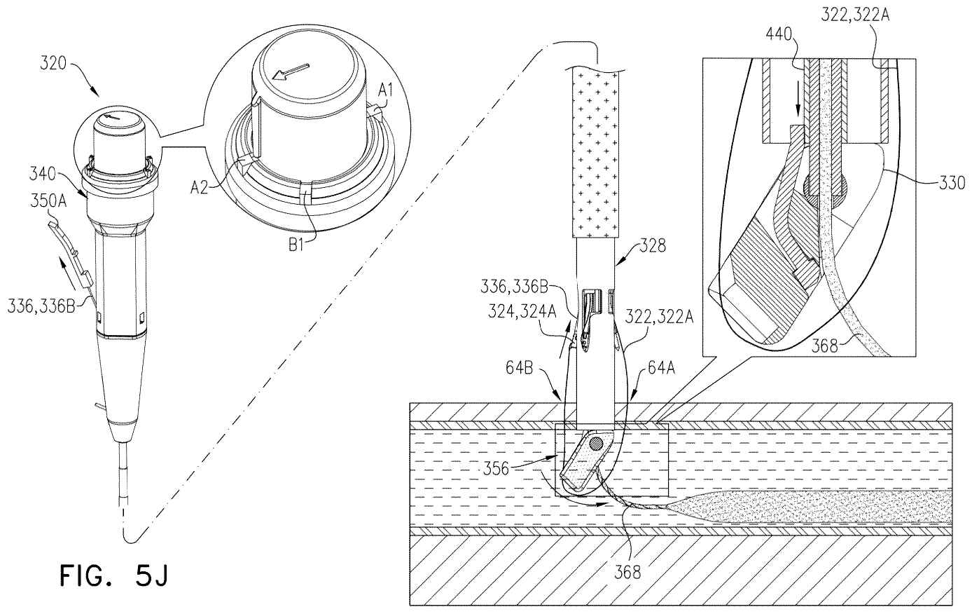

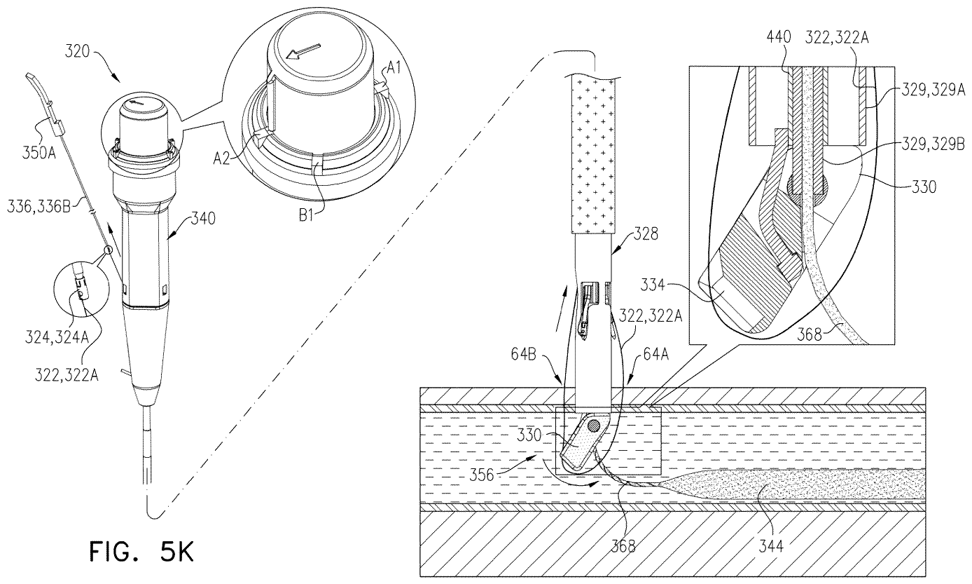

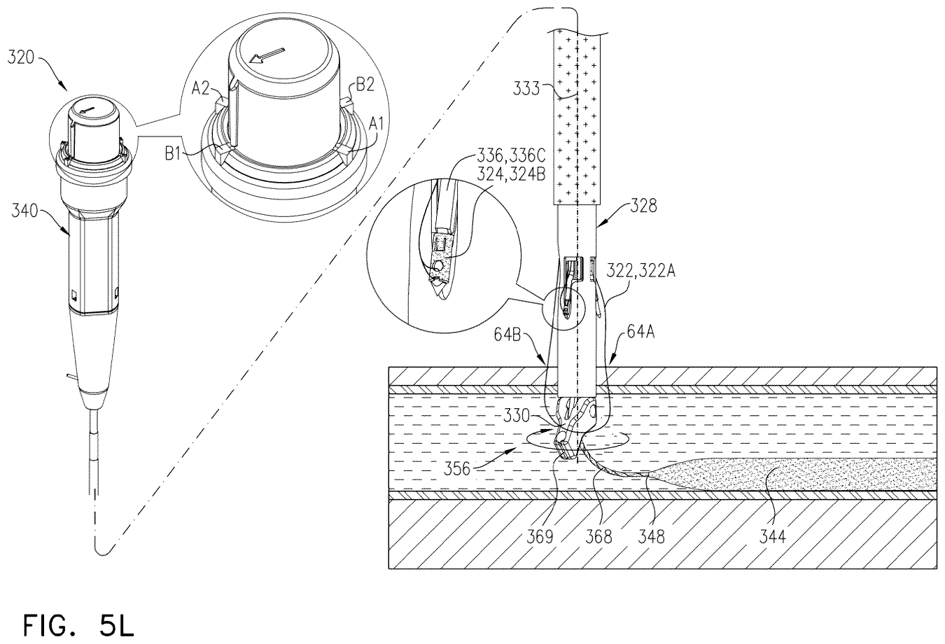

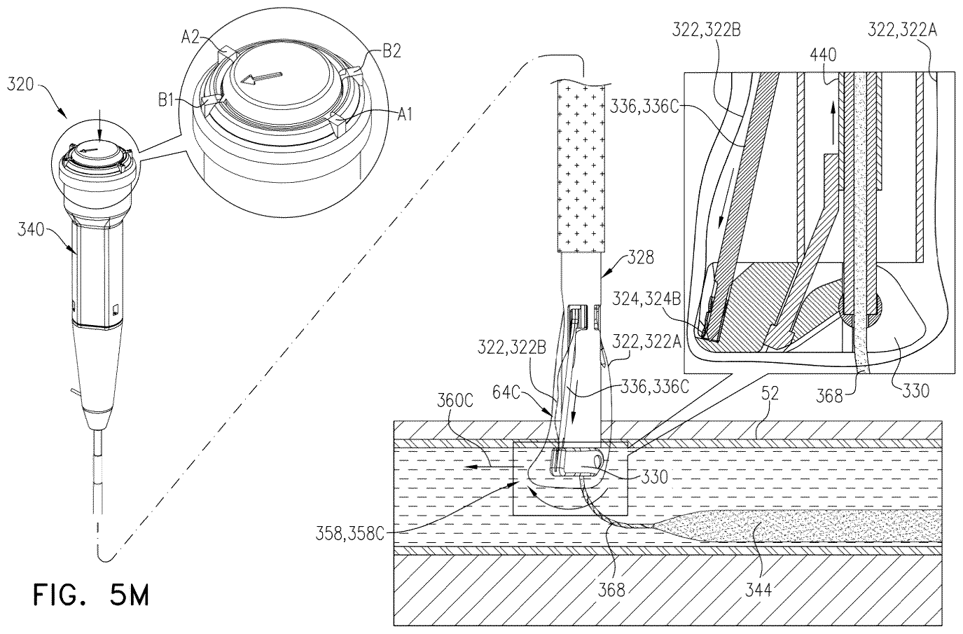

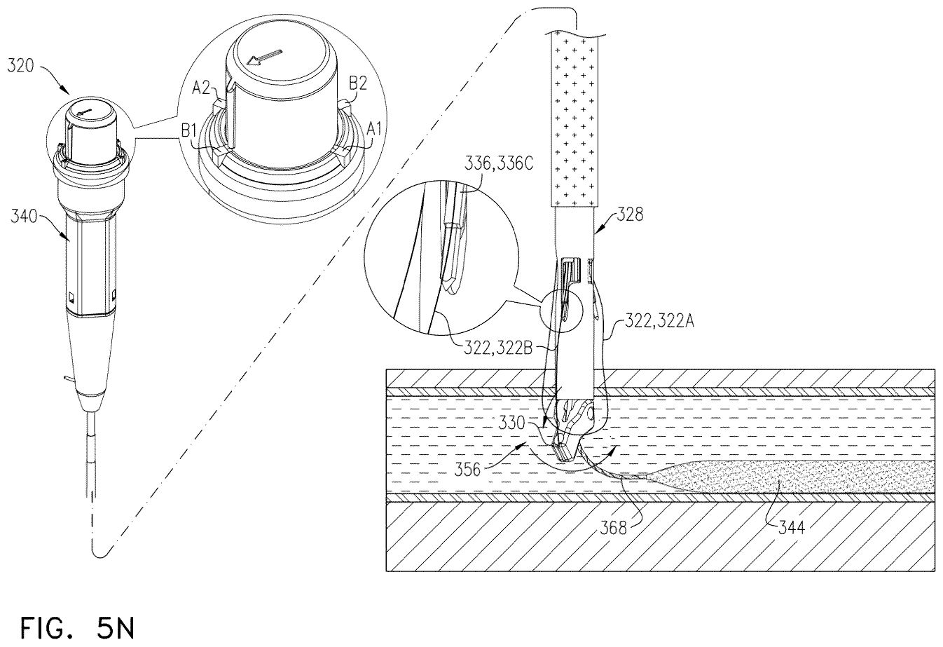

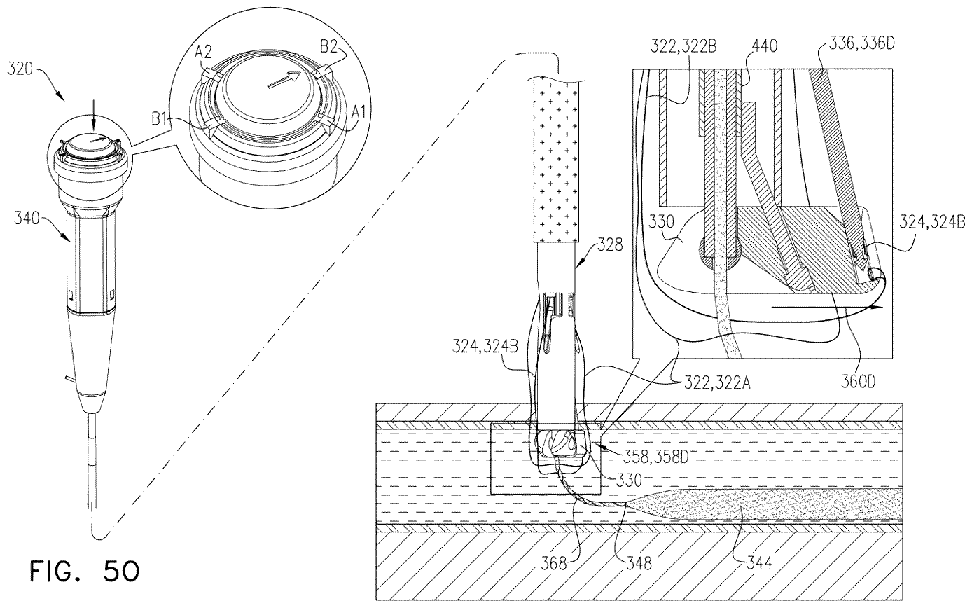

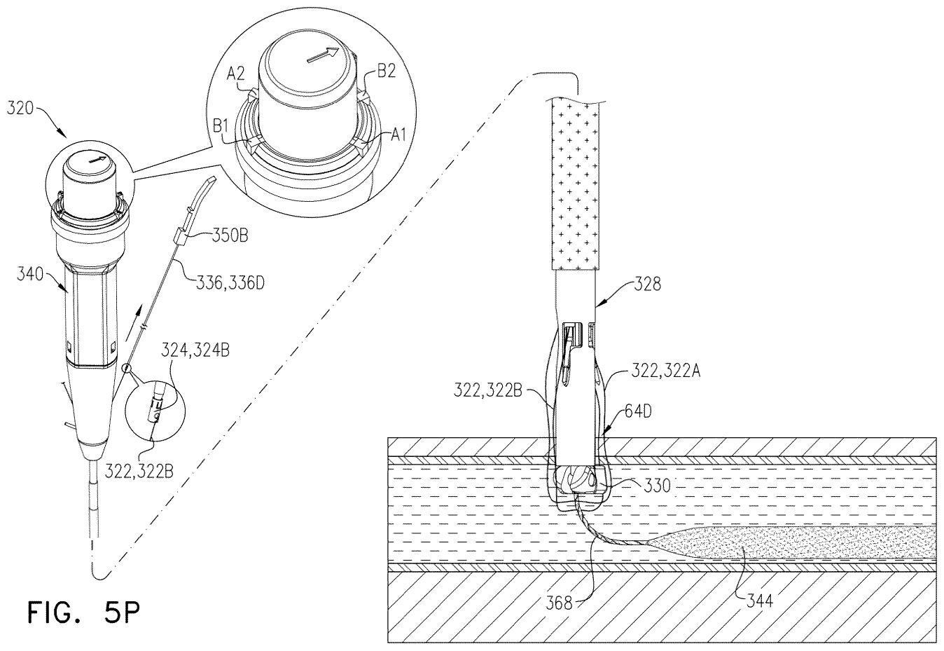

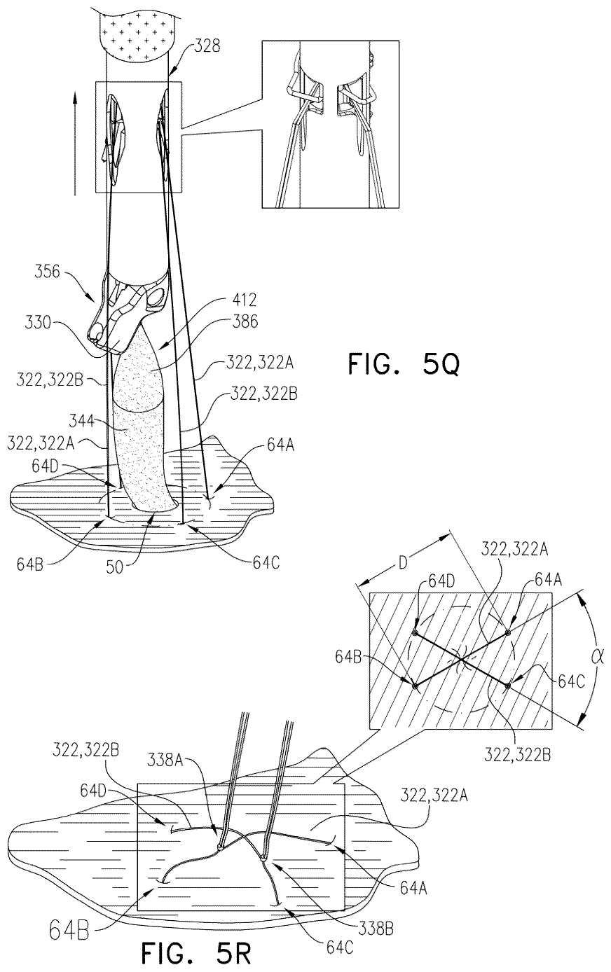

For some applications, elongate support 328 further comprises an inner shaft 329B (labeled in FIGS. 4 and 5C) nested within outer tubular shaft 329A, and suture-positioning support 330 is coupled to inner shaft 329B of elongate support 328.

For some applications, suture-positioning support 330 has proximal and distal end portions 331A and 331B at opposite ends of suture-positioning support 330 (labeled in FIG. 4). Proximal end portion 331A of suture-positioning support 330 is coupled to elongate support 328 (e.g., to inner shaft 329B of elongate support 328, as shown). Distal end portion 331B of suture-positioning support 330 is disposed distally to distal end 406 of outer tubular shaft 329A.

In some applications of the present invention, closure device 320 comprises an elongate flexible dilator connector 368, which couples proximal end 348 of dilator 344 to distal end portion 332 of elongate support 328 so as to allow movement of distal end portion 332 of elongate support 328 with respect to proximal end 348 of dilator 344, optionally with one or more of the above-mentioned degrees of freedom. Typically, proximal end 348 of dilator 344 and distal end portion 332 of elongate support 328 are connected by dilator connector 368 during manufacture of closure device 320, and remain connected throughout ordinary use of closure device 320. Thus, dilator connector 368 typically permanently connects proximal end 348 of dilator 344 to distal end portion 332 of elongate support 328, without enabling disconnection during ordinary use of closure device 320. Optionally, elongate flexible dilator connector 368 may alternatively be referred to as an elongate flexible dilator connecting member, an elongate flexible dilator coupling member, or an elongate flexible dilator linking member.

For some applications, dilator connector 368 and dilator 344 comprise separate respective elements coupled together. Alternatively, dilator connector 368 and dilator 344 are integrally formed from a single element.

For some applications, dilator connector 368 comprises a cable, a cord, a wire, a string, or a tube.

For some applications, dilator connector 368 comprises a metal (e.g., Nitinol or stainless steel) or a polymer.

For some applications, dilator connector 368 has a length of at least 0.5 cm (e.g., at least 1 cm), no more than 5 cm, and/or 0.5-5 cm, e.g., 1-5 cm. (Optionally, dilator connector 368 is anchored within at least a proximal portion of dilator 344 and/or within at least a distal portion of elongate support 328 (e.g., within inner shaft 329B of elongate support 328), such as shown in 5C; in this case, the length of dilator connector 368 includes only the exposed portion of the dilator connector outside the dilator and the elongate support.)

For some applications, a flexural rigidity of dilator 344 is greater than a flexural rigidity of dilator connector 368.

For some applications, a material of dilator 344 is harder than a material of dilator connector 368.

In some applications of the present invention (such as labeled in FIG. 4), an average outer diameter D1 of dilator connector 368 is:
less than 20% of a greatest outer diameter D2 of dilator 344, such as less than 10% of D2,
at least 3% of the greatest outer diameter D2 of dilator 344, such as at least 5% of D2, and/or
at least 0.2 mm, no more than 0.8 mm, and/or 0.2-0.8 mm, e.g., 0.2-0.5 mm.

For some applications, dilator connector 368 is rotationally fixed to distal end portion 332 of elongate support 328 and proximal end 348 of dilator 344. Dilator connector 368 couples proximal end 348 of dilator 344 to distal end portion 332 of elongate support 328 so as to allow rotation of distal end portion 332 of elongate support 328 about distal-support central longitudinal axis 333 without corresponding rotation of dilator 344 about a proximal-dilator central longitudinal axis 418 of dilator 344 of a proximal end portion 412 of dilator 344 that includes proximal end 348 of dilator 344 (labeled in FIGS. 3A-B).

For some applications, suture-positioning support 330 is configured to assume:
a delivery position 356, in which a suture-positioning support axis 335 of suture-positioning support 330 (a) forms a first angle α (alpha) of less than 45 degrees, e.g., less than 30 degrees, with distal-support central longitudinal axis 333 of distal end portion 332 of elongate support 328, such as shown in FIGS. 1A, 2A-D, and 4, and labeled in FIG. 4, or (b) is parallel with distal-support central longitudinal axis 333 (configuration not shown), and
one or more deployed positions 358, in which suture-positioning support 330 is laterally extended with respect to distal end portion 332 of elongate support 328 such that suture-positioning support axis 335 forms a second angle β (beta) of at least 60 degrees, e.g., at least 75 degrees, such as 90 degrees with distal-support central longitudinal axis 333, such as shown in FIGS. 3A-B, and labeled in FIG. 3A.

As used in the present application, including in the claims and the Inventive Concepts, an angle between two elements is the smaller of the two supplementary angles between the two elements, or equals 90 degrees if the two elements are perpendicular.

For some applications, suture-positioning support 330 is disposed distally beyond a distal end 346 of elongate support 328 (labeled in FIGS. 2A and 3B). Typically, but not necessarily, this is the case both when suture-positioning support 330 is in the above-mentioned delivery position 356 and in the above-mentioned one or more deployed positions 358.

Reference is made to FIGS. 2C and 3A. For some applications, suture-positioning support 330 is shaped so as to define a passage 372 therethrough. Dilator connector 368 is disposed passing through passage 372. Passage 372 is typically shaped so as to allow the one or more of the above-mentioned degrees of freedom of movement of distal end portion 332 of elongate support 328 with respect to proximal end 348 of dilator 344, typically both when suture-positioning support 330 is in delivery position 356, such as shown in FIG. 2C, and when suture-positioning support 330 is the one or more deployed positions 358, such as shown in FIG. 3A.

For some applications, passage 372 is shaped so as to define an indentation 374 that slants toward distal end portion 331B of suture-positioning support 330. Indentation 374 may be shaped to receive dilator connector 368 when suture-positioning support 330 is in delivery position 356, such as shown in FIG. 2C, so as to avoid interfering with the freedom of movement of dilator connector 368.

(The configurations of suture-positioning support 330 described hereinbelow with reference to FIGS. 11A-F and 12A-E may also implement these features; optionally, passage 372 may be defined between partial spherical surfaces 650.)

Reference is still made to FIGS. 1A-4 and is additionally made to FIGS. 5A-R, which are schematic illustrations of a method for suturing puncture 50 through wall 52 of hollow anatomical structure 54 using closure device 320, in accordance with an application of the present invention. Although hollow anatomical structure 54 is illustrated as a blood vessel 54, the method may alternatively be performed on other hollow anatomical structures, such as a body cavity, e.g., an abdominal cavity, mutatis mutandis, in which case closure device 320 may be configured to close an endoscopic puncture through a wall of the body cavity. Typically, the surgeon performed the medical procedure that made puncture 50 using the Selinger technique.

As shown in FIG. 5A, a guidewire 390 is inserted through puncture 50 and into the blood vessel 54.

As shown in FIG. 5B, elongate dilator 344 is advanced over guidewire 390 and through puncture 50 and into blood vessel 54. Dilator 344 is shaped so as to define a guidewire channel through which the guidewire passes during the advancement. Typically, the guidewire channel has a distal opening 392 (labeled in FIG. 1A) through a distal end of dilator 344 and a proximal opening 394 through a lateral wall of dilator 344 near proximal end 348 of dilator 344 (labeled in FIGS. 1A and 5B). Optionally, the guidewire channel has a diameter of at least 0.8 mm, no more than 1.2 mm, and/or 0.8-1.2 mm. Guidewire 390 is removed from blood vessel 54.

As shown in FIG. 5C, distal end portion 332 of elongate support 328 is inserted through puncture 50 and into hollow anatomical structure 54 (e.g., the blood vessel), typically while suture-positioning support 330 is in delivery position 356.

Reference is made to FIGS. 5C-D. As described hereinabove with reference to FIGS. 1A-4, for some applications elongate support 328 comprises outer tubular shaft 329A having a distal end 406 (labeled in FIG. 3B). In some applications of the present invention, closure device 320 further comprises a sheath 410 that covers: distal end 406 of outer tubular shaft 329A, suture-positioning support 330, dilator connector 368, and proximal end portion 412 of dilator 344 that includes proximal end 348 of dilator 344, such as shown in FIG. 5C, in which sheath 410 is shown distally advanced in a distally advanced position. Sheath 410 thus may facilitate atraumatic insertion of distal end 406 of outer tubular shaft 329A and suture-positioning support 330 through puncture 50, despite the narrowing of closure device 320 along dilator connector 368 and atraumatic proximal tip 386 of dilator 344.

After distal end 406 of outer tubular shaft 329A and suture-positioning support 330 have been inserted through puncture 50 and into blood vessel 54, sheath 410 is proximally withdrawable along outer tubular shaft 329A so as to expose suture-positioning support 330, dilator connector 368, and proximal end portion 412 of dilator 344, such as shown in FIG. 5D. For example, control handle 340 may comprise a sheath-control user control 430, such as described hereinbelow with reference to FIGS. 6A-B. (For clarity of illustration, sheath 410 is shown proximally withdrawn in FIGS. 1A-B, even though sheath 410 is in practice not withdrawn until after insertion of outer tubular shaft 329A and suture-positioning support 330 through puncture 50 and into blood vessel 54.)

For some applications, sheath 410 is flexible enough to change diameter, e.g., to accommodate slight lateral protrusion of suture-positioning support 330 even while suture-positioning support 330 is in delivery position 356, such as shown in FIG. 5C. For some applications, sheath 410 comprises a material selected from the group of polymeric materials consisting of: silicone and polyether block amide (PEBA).

As shown in FIG. 5C, in some applications of the present invention, distal end portion 332 of elongate support 328 is inserted of closure device 320 through puncture 50 and into blood vessel 54, while distal-support central longitudinal axis 333 of distal end portion 332 of elongate support 328 forms an angle ε (epsilon) of less than 30 degrees with respect to a blood-vessel central longitudinal axis 420 of blood vessel 54 at a site 422 of puncture 50. Thereafter, as shown in FIG. 5E, distal end portion 332 of elongate support 328 is moved with respect to blood vessel 54 to define a second angle θ (theta) between distal-support central longitudinal axis 333 and blood-vessel central longitudinal axis 420, such as shown in FIG. 5E.

For some applications:
the first angle ε (epsilon) is less than 30 degrees and the second angle θ (theta) is 45-90 degrees, such as 60-90 degrees, e.g., 75-90 degrees, such as 90 degrees, and/or
the second angle θ (theta) is at least 15 degrees greater than the first angle ε (epsilon), such as at least 30 degrees, e.g., at least 45 degrees, such as at least 60 degrees, greater than the first angle γ (gamma).

As also shown in FIGS. 5C and 5E, in some applications of the present invention, distal end portion 332 of elongate support 328 defines distal-support central longitudinal axis 333. Proximal end portion 412 of dilator 344, which includes proximal end 348 of dilator 344, defines proximal-dilator central longitudinal axis 418. Closure device 320 is configured to allow distal end portion 332 of elongate support 328 to move with respect to proximal end 348 of dilator 344 from defining a first angle γ (gamma) (as shown in FIG. 5D) to defining a second angle δ (delta) (as shown in FIG. 5E) between distal-support central longitudinal axis 333 and proximal-dilator central longitudinal axis 418.

For some applications:
the first angle γ (gamma) is less than 30 degrees and the second angle δ (delta) is 45-90 degrees, such as 60-90 degrees, e.g., 75-90 degrees, such as 90 degrees, and/or
the second angle δ (delta) is at least 15 degrees greater than the first angle γ (gamma), such as at least 30 degrees, e.g., at least 45 degrees, such as at least 60 degrees, greater than the first angle γ (gamma).

For some applications in which closure device 320 comprises elongate flexible dilator connector 368, the elongate flexible dilator connector couples proximal end 348 of dilator 344 to distal end portion 332 of elongate support 328 so as to allow the above-described movement.

For some applications, distal end portion 332 of elongate support 328 is moved with respect to proximal end 348 of dilator 344 without causing distal end portion 332 of elongate support 328 to directly or indirectly apply a force to proximal end 348 of dilator 344.

For some applications, distal end portion 332 of elongate support 328 is moved with respect to proximal end 348 of dilator 344 without bending dilator 344.

For some applications, distal end portion 332 of elongate support 328 is moved with respect to proximal end 348 of dilator 344 without deforming dilator 344.

For some applications, distal end portion 332 of elongate support 328 is moved with respect to proximal end 348 of dilator 344 with at least two degrees of freedom, such as:
with at least one rotational degree of freedom, such as at least two rotational degrees of freedom, e.g., with three rotational degrees of freedom,
with at least one translational degree of freedom, such as a translational degree of freedom along distal-support central longitudinal axis 333, and/or with at least two translational degrees of freedom, e.g., with three translational degrees of freedom, and/or
with at least four degrees of freedom, e.g., with six degrees of freedom.

For some applications, distal end portion 332 of elongate support 328 is moved with respect to proximal end 348 of dilator 344 without causing dilator 344 to apply a force to wall 52 of blood vessel 54.

For some applications, dilator 344 is laterally moved while distal end portion 332 of elongate support 328 remains stationary with respect to puncture 50.

For any of the applications described above, elongate flexible dilator connector 368, described hereinabove with reference to FIGS. 1A-4, couples proximal end 348 of dilator 344 to distal end portion 332 of elongate support 328 so as to allow the described movement.

For some of these applications, dilator connector 368 is rotationally fixed to distal end portion 332 of elongate support 328 and proximal end 348 of dilator 344, and distal end portion 332 of elongate support 328 is rotated about distal-support central longitudinal axis 333 without corresponding rotation of dilator 344 about proximal-dilator central longitudinal axis 418. To allow this rotation, dilator connector 368 typically twists and/or bunches up, such as shown in FIG. 5H.

As shown in FIG. 5F, suture-positioning support 330 is laterally extended, with respect to distal end portion 332 of elongate support 328, to a first deployed position 358, 358A in which suture-positioning support 330 laterally extends in a first direction 360A from distal end portion 332 of elongate support 328. For example, suture-positioning support 330 may be laterally extended using control handle 340 as described hereinbelow with reference to FIGS. 7A-B.

A first suture 322, 322A is distally advanced through a portion of elongate support 328 and a first wall site 64A of wall 52 and into blood vessel 54, and into suture-positioning support 330.

For some applications, such as shown in the transition between FIG. 5E and FIG. 5F, suture-positioning support 330 is pivotably coupled to distal end portion 332 of elongate support 328 such that suture-positioning support 330 is rotatable about a pivot axis 362 to transition from delivery position 356 to first deployed position 358, 358A. Typically, pivot axis 362 is not coaxial with distal-support central longitudinal axis 333.

Reference is made to FIGS. 1A-D, 4, and 5F-O. For some applications, closure device 320 further comprises:
a ferrule 324, which is coupled to a distal end portion 326 of first suture 322, 322A (labeled in FIG. 3A), such as by being passed (e.g., looped) through an opening defined by a wall of ferrule 324 (such as shown), and/or by welding, knotting, gluing, or another technique (configurations not shown); and
one or more suturing needles 336, which are removably couplable to ferrule 324.

Suture-positioning support 330 is shaped so as to define a ferrule receptacle 334 (labeled in FIGS. 4 and 5G), which is configured to removably receive ferrule 324, such that suture-positioning support 330 is configured to removably receive first suture 322, 322A. For some applications, suture-positioning support 330 is shaped so as to define exactly one ferrule receptacle 334, such as shown. For other applications, suture-positioning support 330 is shaped so as to define two or more ferrule receptacles, such as described hereinabove.

Elongate support 328 defines, through a longitudinal portion of elongate support 328, one or more needle lumens 366 having respective distal lumen openings 367 (labeled in FIG. 4). Needle lumens 366 are shaped so as to direct respective suturing needles 336 out of respective distal lumen openings 367 and toward ferrule receptacle 334 during distal advancement of respective suturing needles 336, optionally removably coupled to ferrule 324. To this end, suturing needles 336 are typically sufficiently flexible to be bent and directed by needle lumens 366 in the proper direction of advancement, such as perhaps best seen in FIG. 4.

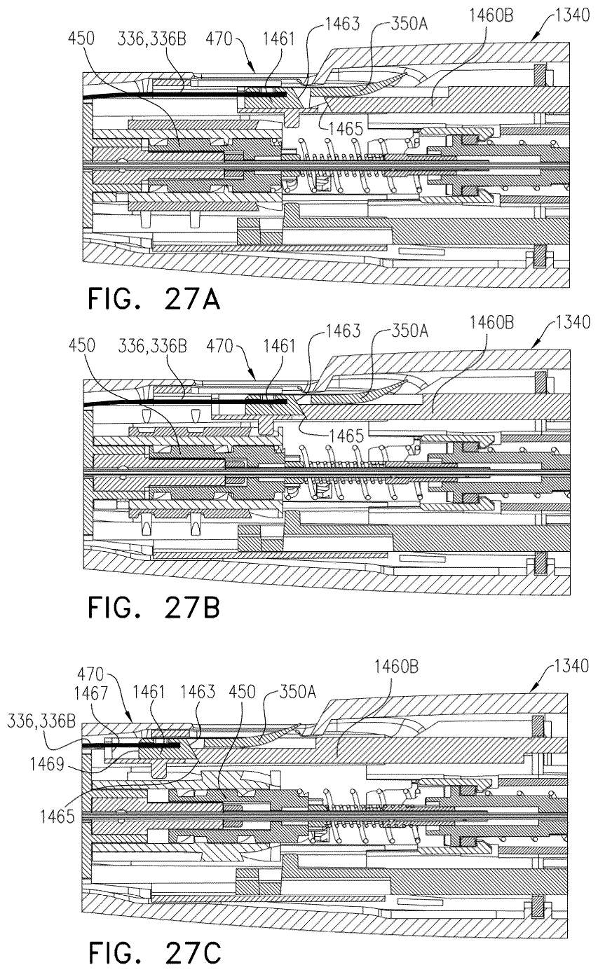

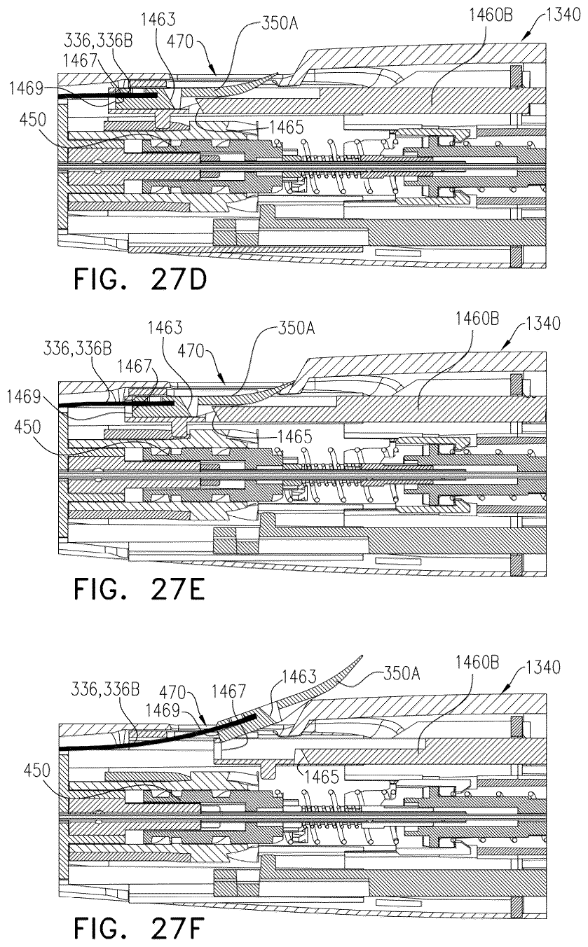

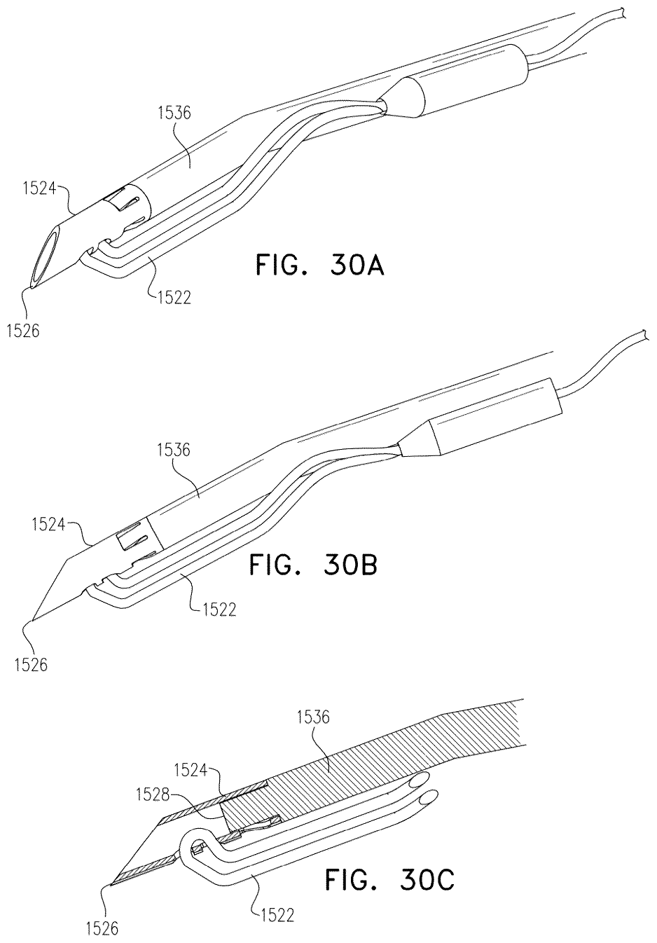

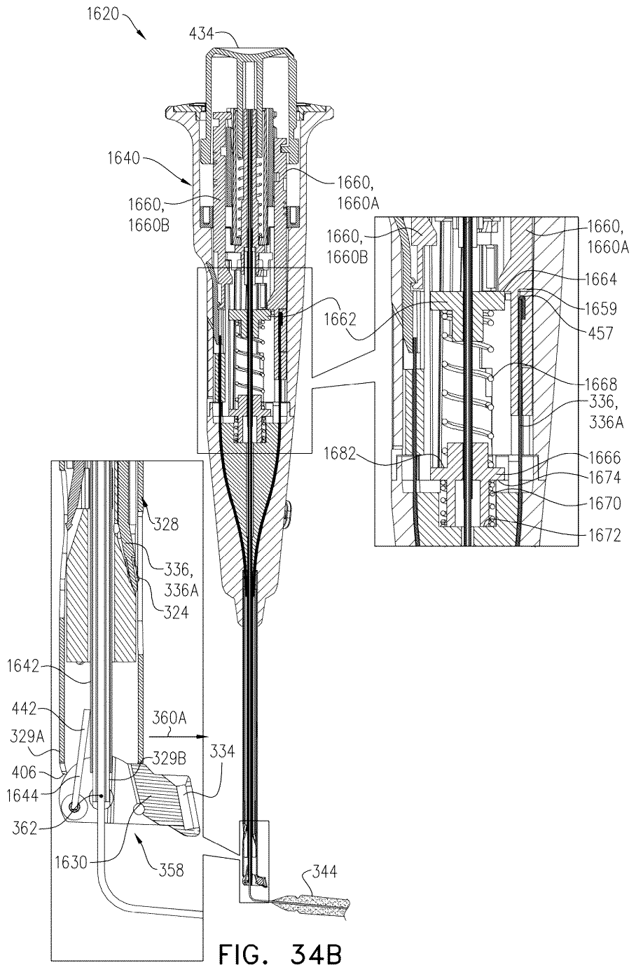

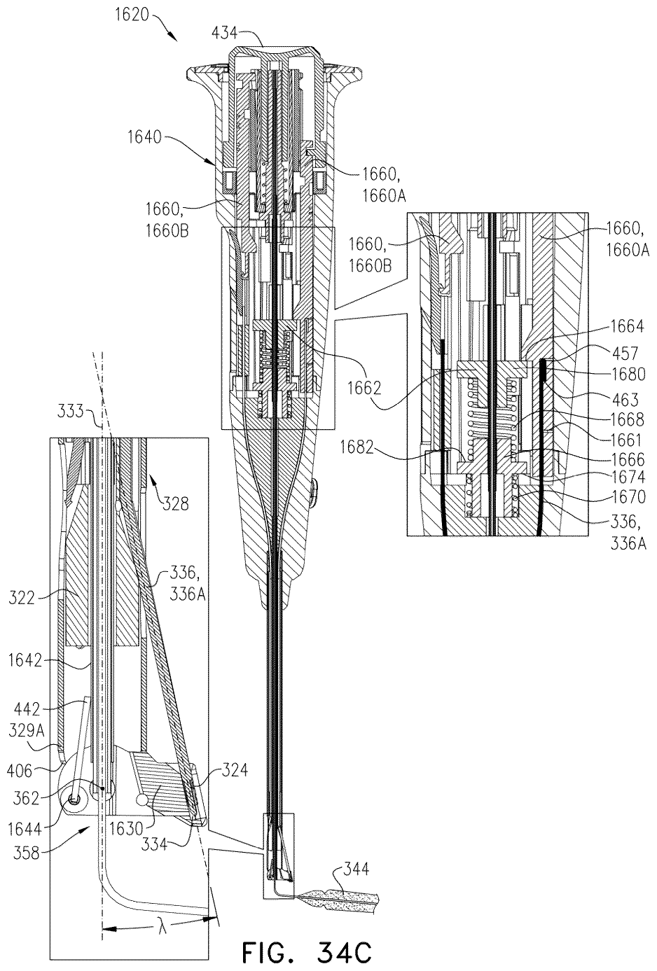

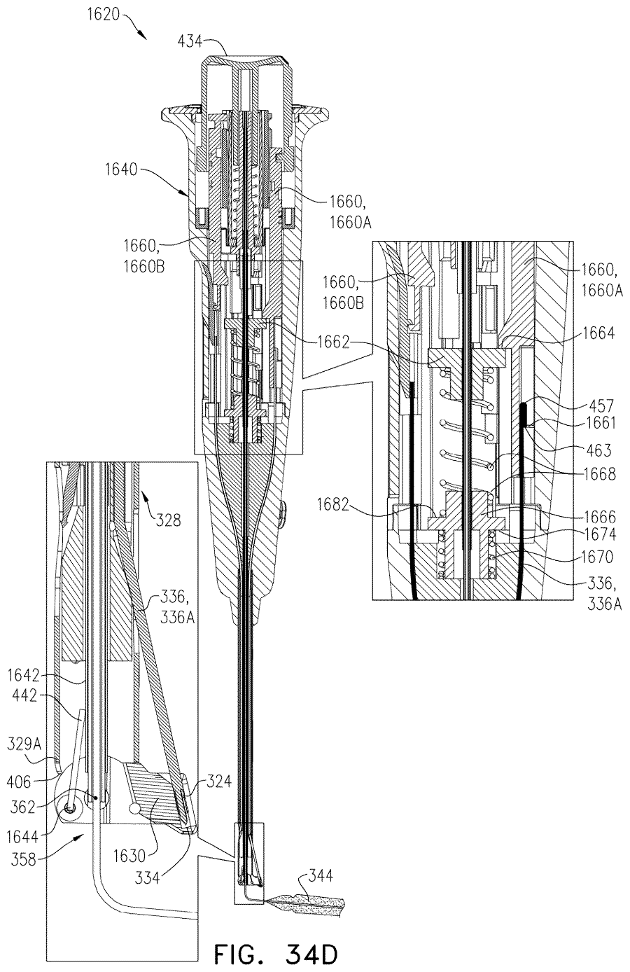

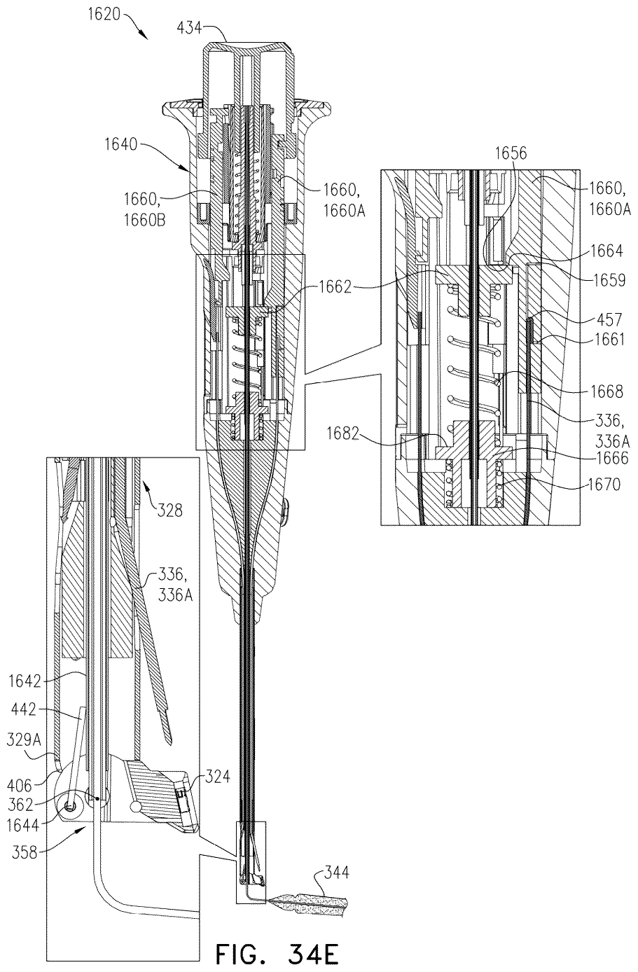

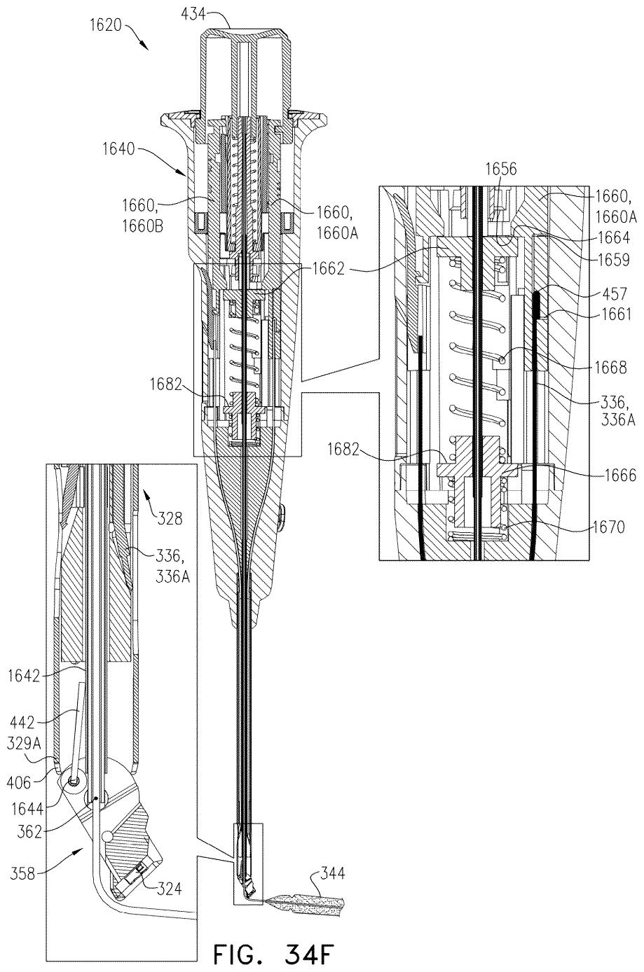

Reference is made to FIG. 5F. For some applications, first suture 322, 322A is distally advanced into suture-positioning support 330 by distally advancing a first ferrule-advancing suturing needle 336, 336A of suturing needles 336 through first wall site 64A of wall 52 and into blood vessel 54, while first ferrule-advancing suturing needle 336, 336A is removably coupled to ferrule 324, such that closure device 320 directs ferrule 324 into ferrule receptacle 334 defined by suture-positioning support 330 and ferrule receptacle 334 removably receives ferrule 324 while suture-positioning support 330 is in first deployed position 358, 358A. (Suture-positioning support 330 thus removably engages first suture 322, 322A.) For some applications, suturing needles 336 have sharp distal tips, in which case ferrule-advancing suturing needles 336 typically protrude out of the distal end of ferrule 324; for other applications, the sharp ferrule techniques described hereinbelow with reference to FIGS. 30A-33 may be used. For example, first ferrule-advancing suturing needle 336, 336A may be distally advanced using control handle 340 as described hereinbelow with reference to FIGS. 7A-B, using control handle 1340 as described hereinbelow with reference to FIGS. 27A-F, or using control handle 1640 as described hereinbelow with reference to FIGS. 34A-F.

Typically, first ferrule-advancing suturing needle 336, 336A is removably coupled to ferrule 324 before the beginning of the procedure, although first ferrule-advancing suturing needle 336, 336A may alternatively become removably coupled to ferrule 324 during the procedure, such as during distal advancement of first ferrule-advancing suturing needle 336, 336A through or out of a first one of needle lumens 366.

For some applications, first ferrule-advancing suturing needle 336, 336A and second ferrule-advancing suturing needle 336, 336C, described hereinbelow, are initially removably disposed entirely within control handle 340, prior to distal advancement of the ferrule-advancing suturing needles, as described herein.

Optionally, the surgeon adjusts an axial position of elongate support 328 such that suture-positioning support 330 touches, or is near, an inner surface of wall 52, as shown in FIG. 5F. The various degrees of freedom of distal end portion 332 of elongate support 328 with respect to proximal end 348 of dilator 344 may facilitate this adjustment of the axial position of elongate support 328.

For some applications in which closure device 320 comprises sheath 410, such as described hereinabove with reference to FIGS. 5C-D, sheath 410 covers distal end 406 of outer tubular shaft 329A, suture-positioning support 330, dilator connector 368, proximal end portion 412 of dilator 344, and distal lumen opening 367. (Distal lumen opening 367 is sometimes introduced into the body, albeit not into the blood vessel.) Sheath 410 is proximally withdrawable along outer tubular shaft 329A so as to expose suture-positioning support 330, dilator connector 368, proximal end portion 412 of dilator 344, and distal lumen opening 367, such as shown in FIG. 5D.

For these applications, such as shown in FIG. 5B, distal end portion 332 of elongate support 328 is inserted through puncture 50 and into blood vessel 54 while sheath 410 covers: distal end 406 of outer tubular shaft 329A, suture-positioning support 330, dilator connector 368, and proximal end portion 412 of dilator 344. The method further comprises, after inserting distal end portion 332 of elongate support 328 and before laterally extending suture-positioning support 330: proximally withdrawing sheath 410 along outer tubular shaft 329A so as to expose suture-positioning support 330, dilator connector 368, proximal end portion 412 of dilator 344, and distal lumen opening 367, if provided and covered by sheath 410. For example, control handle 340 may comprise sheath-control user control 430, such as described hereinbelow with reference to FIGS. 6A-B.

As shown in FIG. 5G, first ferrule-advancing suturing needle 336, 336A is proximally withdrawn from ferrule 324 and hollow anatomical structure 54, while leaving ferrule 324 within ferrule receptacle 334. For example, ferrule 324 may be held in ferrule receptacle 334 as first ferrule-advancing suturing needle 336, 336A is proximally withdrawn from ferrule 324 by one or more of the following features:
ferrule receptacle 334 may have a smaller cross-sectional area than that of ferrule 324; and/or
ferrule receptacle 334 may be shaped so as to define a slit 369 (labeled in FIGS. 2B, 5L, 11A-F, and 12A-F), e.g., through a wall of suture-positioning support 330, which, for example, allows ferrule receptacle 334 to expand slightly as ferrule 324 is inserted into the receptacle, applying friction and a spring-like effect to the ferrule; slit 369 may also provide the functionality described hereinbelow with reference to FIGS. 12A-F.

For example, first ferrule-advancing suturing needle 336, 336A may be proximally withdrawn using control handle 340 as described hereinbelow with reference to FIG. 7D.

Also as shown in FIG. 5G, suture-positioning support 330 is transitioned from first deployed position 358, 358A back to delivery position 356. (This position is called a “delivery” position, even though suture-positioning support 330 is not again delivered at this step, because the position is typically the same position as during initial delivery of suture-positioning support 330.) Optionally, as shown in the transition shown in FIG. 5G, suture-positioning support 330 is transitioned from first deployed position 358, 358A back to delivery position 356 by rotating suture-positioning support 330 about pivot axis 362. For example, suture-positioning support 330 may be transitioned from first deployed position 358, 358A back to delivery position 356 using control handle 340 as described hereinbelow with reference to FIG. 7E.

As shown in FIG. 5H, suture-positioning support 330, while in delivery position 356, is rotated about distal-support central longitudinal axis 333. Rotating suture-positioning support 330 while in delivery position 356 may help minimize the footprint of the device in the blood vessel. For some of these applications, the rotation is achieved by rotation of one or more of the shafts of elongate support 328, with respect to control handle 340, about distal-support central longitudinal axis 333. For example, elongate support 328 may comprise outer tubular shaft 329A and an inner shaft 329B, nested within outer tubular shaft 329A, and closure device 320 may be configured to rotate inner shaft 329B while holding outer tubular shaft 329A fixed with respect to control handle 340, such as shown. For example, suture-positioning support 330 may be rotated about distal-support central longitudinal axis 333 using control handle 340 (e.g., support-and-needle user control 434 thereof, described hereinbelow) as described hereinbelow with reference to FIG. 7F.

Alternatively, closure device 320 may be configured to rotate outer tubular shaft 329A with respect to control handle 340 (configuration not shown).

Further alternatively, elongate support 328 may comprise only a single shaft, and closure device 320 may be configured to rotate the single shaft with respect to control handle 340 (configuration not shown). Further alternatively, the rotation is achieved by rotation of elongate support 328 by rotation of control handle 340 about distal-support central longitudinal axis 333 by the surgeon, such as described in the '1800 publication for the transition of suture-positioning support 130 of closure device 220, with reference to FIG. 8D through FIG. 8F thereof, mutatis mutandis.

The one or more rotational degrees of freedom of distal end portion 332 of elongate support 328 with respect to proximal end 348 of dilator 344 may facilitate this adjustment of the axial position of elongate support 328, particularly in configurations in which dilator connector 368 is rotationally fixed to distal end portion 332 of elongate support 328 and proximal end 348 of dilator 344.

As shown in the transition between FIG. 5H and FIG. 5I, suture-positioning support 330 is again laterally extended, with respect to distal end portion 332 of elongate support 328, to a second deployed position 358, 358B in which suture-positioning support 330 laterally extends in a second direction 360B from distal end portion 332 of elongate support 328, second direction 360B different from first direction 360A. For example, first direction 360A and second direction 360B may be separated by an angle of 120-240 degrees, such as 175-85 degrees, e.g., 180 degrees, around distal-support central longitudinal axis 333 of distal end portion 332 of elongate support 328. For example, suture-positioning support 330 may be laterally extended using control handle 340 as described hereinbelow with reference to FIG. 7G.

As shown in FIG. 5J, while suture-positioning support 330 is in second deployed position 358B, ferrule 324 is proximally withdrawn from ferrule receptacle 334 and out of hollow anatomical structure 54 via a second wall site 64B of wall 52, so as to proximally withdraw a portion of first suture 322, 322A, including distal end portion 326 thereof, out of hollow anatomical structure 54 via second wall site 64B. The portion of first suture 322, 322A drawn out of hollow anatomical structure 54 via second wall site 64B is secured to another portion of first suture 322, 322A, for example by a first pre-tied knot 338A, such as described hereinbelow with reference to FIGS. 1B-D.

In some applications of the present invention, suturing needles 336 of closure device 320 further comprise a first ferrule-withdrawing suturing needle 336, 336B, which is couplable to ferrule 324. As shown in FIG. 5I, closure device 320 is configured to direct first ferrule-withdrawing suturing needle 336, 336B to ferrule 324 during distal advancement of first ferrule-withdrawing suturing needle 336, 336B. For example, first ferrule-withdrawing suturing needle 336, 336B may be distally advanced using control handle 340 as described hereinbelow with reference to FIG. 7G.

For some applications, first ferrule-withdrawing suturing needle 336, 336B and second ferrule-withdrawing suturing needle 336, 336D, described hereinbelow, are initially removably disposed entirely within control handle 340, prior to distal advancement of the ferrule-withdrawing suturing needles, as described herein.

Closure device 320 is configured such that after the distal advancement of first ferrule-withdrawing suturing needle 336, 336B to ferrule 324, proximal withdrawal of first ferrule-withdrawing suturing needle 336, 336B coupled to ferrule 324 removes ferrule 324 from ferrule receptacle 334, such as shown in FIG. 5J. For example, first ferrule-withdrawing suturing needle 336, 336B may be proximally withdrawn using control handle 340 as described hereinbelow with reference to FIG. 7H.

For some applications, such as shown in FIGS. 5J-K, after first ferrule-withdrawing suturing needle 336, 336B has been proximally withdrawn within control handle 340, first ferrule-withdrawing suturing needle 336, 336B is removed from control handle 340, such as by using a needle handle 350A that is coupled to a proximal end of first ferrule-withdrawing suturing needle 336, 336B. As mentioned above, at this stage of the procedure, first ferrule-withdrawing suturing needle 336, 336B is coupled to first suture 322, 322A via first ferrule 324, 324A. Therefore, removal of first ferrule-withdrawing suturing needle 336, 336B from control handle 340 pulls a portion of first suture 322, 322A out of the control handle. This removal of first ferrule-withdrawing suturing needle 336, 336B from control handle 340 is described in more detail hereinbelow with reference to FIGS. 7A-H, in one configuration, and with reference to FIGS. 27A-F, in another configuration. (As shown, needle handle 350A is typically moveable separately from, separate and discrete from, and/or not coupled to support-and-needle user control 434, described hereinbelow.)

For some applications, first ferrule-withdrawing suturing needle 336, 336B, including a distal end portion thereof and needle handle 350A, is initially removably disposed entirely within control handle 340, prior to distal advancement of the first ferrule-withdrawing suturing needle, as described herein. Similarly, for some applications, second ferrule-withdrawing suturing needle 336, 336D (described hereinbelow), including a distal end portion thereof and needle handle 350B (described hereinbelow), is initially removably disposed entirely within control handle 340, prior to distal advancement of the second ferrule-withdrawing suturing needle, as described herein.

Reference is made to FIGS. 5I-J. For some applications, ferrule 324 is proximally withdrawn from ferrule receptacle 334 and out of hollow anatomical structure 54 while suture-positioning support 330 is in second deployed position 358B by:
as shown in FIG. 5I, distally advancing first ferrule-withdrawing suturing needle 336, 336B through second wall site 64B and into hollow anatomical structure 54, such that closure device 320 directs first ferrule-withdrawing suturing needle 336, 336B to ferrule 324 and first ferrule-withdrawing suturing needle 336, 336B becomes coupled to ferrule 324, and
as shown in FIG. 5J, proximally withdrawing first ferrule-withdrawing suturing needle 336, 336B from hollow anatomical structure 54 via second wall site 64B, so as to remove ferrule 324 from ferrule receptacle 334 and to proximally withdraw distal end portion 326 of first suture 322A out of hollow anatomical structure 54 via second wall site 64B.

Optionally, the surgeon adjusts an axial position of elongate support 328 (e.g., by slightly proximally withdrawing elongate support 328) such that suture-positioning support 330 touches, or is near, an inner surface of wall 52, as shown in FIG. 5I. The various degrees of freedom of distal end portion 332 of elongate support 328 with respect to proximal end 348 of dilator 344 may facilitate this adjustment of the axial position of elongate support 328.

Ferrule 324 is shaped so as to define a ferrule lumen 370 (labeled in FIGS. 4 and 5F). Optionally, ferrule 324 is shaped so as to define the one or more tabs 373 that are biased to protrude radially inward within ferrule lumen 370 and to engage first ferrule-withdrawing suturing needle 336, 336B upon the insertion of first ferrule-withdrawing suturing needle 336, 336B into a first end opening 371A of ferrule lumen 370, so as to inhibit withdrawal of first ferrule-withdrawing suturing needle 336, 336B from ferrule lumen 370, such as shown in FIG. 5I. Typically, ferrule lumen 370 also has a second end opening 371B at the opposite end of the lumen from first end opening 371A.

Reference is made to FIGS. 4 and 5I. For some applications, elongate support 328 defines a first needle lumen 366, 366A and a second needle lumen 366, 366B through respective longitudinal portions of elongate support 328 (labeled in FIG. 4). First needle lumen 366, 366A and second needle lumen 366, 366B are shaped so as to direct first ferrule-advancing suturing needle 336, 336A and first ferrule-withdrawing suturing needle 336, 336B toward ferrule receptacle 334 during the distal advancement of first ferrule-advancing suturing needle 336, 336A and the distal advancement of first ferrule-withdrawing suturing needle 336, 336B, respectively, such as shown in FIGS. 5F and 5I, respectively.

For some applications, first ferrule-advancing suturing needle 336, 336A is distally advanced through first wall site 64A and into hollow anatomical structure 54 while a portion of first ferrule-advancing suturing needle 336, 336A is disposed within first needle lumen 366, 366A, such that first needle lumen 66, 366A directs first ferrule-advancing suturing needle 336, 336A toward ferrule receptacle 334 while suture-positioning support 330 is in first deployed position 358, 358A, such as shown in FIG. 5F. First ferrule-withdrawing suturing needle 336, 336B is distally advanced through second wall site 64B and into hollow anatomical structure 54 while a portion of first ferrule-withdrawing suturing needle 336, 336B is disposed within second needle lumen 366, 366B, such that second needle lumen 366, 366B directs first ferrule-withdrawing suturing needle 336, 336B toward ferrule receptacle 334 while suture-positioning support 330 is in second deployed position 358, 358B, such as shown in FIG. 5I.

Also as shown in FIG. 5J, suture-positioning support 330 is transitioned from second deployed position 358, 358B back to delivery position 356, optionally by rotating suture-positioning support 330 about pivot axis 362. For example, suture-positioning support 330 may be transitioned from second deployed position 358, 358B back to delivery position 356 using control handle 340 as described hereinbelow with reference to FIG. 7H.

As shown in FIG. 5L, suture-positioning support 330 is rotated about distal-support central longitudinal axis 333, optionally as described hereinabove with reference to FIG. 5H. For example, suture-positioning support 330 may be rotated about distal-support central longitudinal axis 333 using control handle 340 as described hereinbelow with reference to FIG. 7F.

As shown in the transition between FIG. 5L and FIG. 5M, suture-positioning support 330 is again laterally extended, with respect to distal end portion 332 of elongate support 328, to a third deployed position 358, 358C in which suture-positioning support 330 laterally extends in a third direction 360C from distal end portion 332 of elongate support 328, third direction 360C different from first direction 360A and second direction 360B. For example, third direction 360C and first direction 360A may be separated by an angle of 30-150 degrees, e.g., 115-25 degrees, e.g., 120 degrees, around distal-support central longitudinal axis 333 of distal end portion 332 of elongate support 328. For example, suture-positioning support 330 may be laterally extended using control handle 340 as described hereinbelow with reference to FIGS. 7A-B regarding lateral extension to first deployed position 358, 358A, mutatis mutandis.

As shown in FIG. 5M, a second suture 322, 322B is distally advanced through a portion of elongate support 328 and a third wall site 64C of wall 52 and into blood vessel 54, and into suture-positioning support 330. Optionally, first and second sutures 322A and 322B are color-coded, i.e., have different colors, to aid the surgeon during the procedure.

Reference is still made to FIG. 5M. For some applications, ferrule 324, described hereinabove with reference to FIGS. 5C-L, is a first ferrule 324, 324A, and closure device 320 further comprises a second ferrule 324, 324B, which is coupled to a distal end portion of second suture 322, 322B, such as by being passed (e.g., looped) through an opening defined by a wall of ferrule 324, 324A (such as shown), and/or by welding, knotting, gluing, or another technique (configurations not shown). Second suture 322, 322B is distally advanced into suture-positioning support 330 by distally advancing a second ferrule-advancing suturing needle 336, 336C of suturing needles 336 through third wall site 64C of wall 52 and into blood vessel 54, while first ferrule-withdrawing suturing needle 336, 336B is removably coupled to second ferrule 324, 324B, such that closure device 320 directs second ferrule 324, 324B into ferrule receptacle 334 defined by suture-positioning support 330 and ferrule receptacle 334 removably receives second ferrule 324, 324B while suture-positioning support 330 is in third deployed position 358, 358C. For example, second ferrule-advancing suturing needle 336, 336C may be distally advanced using control handle 340 as described hereinbelow with reference to FIGS. 7A-B, using control handle 1340 as described hereinbelow with reference to FIGS. 27A-F, or using control handle 1640 as described hereinbelow with reference to FIGS. 34A-F, regarding first ferrule-advancing suturing needle 336, 336A, mutatis mutandis.

Typically, second ferrule-withdrawing suturing needle 336, 336C is removably coupled to second ferrule 324, 324B before the beginning of the procedure, although second ferrule-withdrawing suturing needle 336, 336C may alternatively become removably coupled to second ferrule 324, 324B during the procedure, such as during distal advancement of second ferrule-withdrawing suturing needle 336, 336C through or out of a third one of needle lumens 366. Optionally, the surgeon adjusts an axial position of elongate support 328 such that suture-positioning support 330 touches, or is near, an inner surface of wall 52, as shown in FIG. 5F. The various degrees of freedom of distal end portion 332 of elongate support 328 with respect to proximal end 348 of dilator 344 may facilitate this adjustment of the axial position of elongate support 328.

As shown in the transition between FIG. 5M and FIG. 5N, second ferrule-advancing suturing needle 336, 336C is proximally withdrawn from second ferrule 324, 324B and hollow anatomical structure 54, while leaving second ferrule 324, 324B within ferrule receptacle 334. For example, second ferrule-advancing suturing needle 336, 336C may be proximally withdrawn using control handle 340 as described hereinbelow with reference to FIG. 7D for first ferrule-advancing suturing needle 336, 336A, mutatis mutandis.

Also as shown in the transition between FIG. 5M and FIG. 5N, suture-positioning support 330 is transitioned from third deployed position 358, 358C back to delivery position 356, optionally by rotating suture-positioning support 330 about pivot axis 362. For example, suture-positioning support 330 may be transitioned from third deployed position 358, 358C back to delivery position 356 using control handle 340 as described hereinbelow with reference to FIG. 7E, mutatis mutandis.

As further shown in the transition between FIG. 5M and FIG. 5N, suture-positioning support 330 is rotated about distal-support central longitudinal axis 333, optionally as described hereinabove with reference to FIG. 5H. For example, suture-positioning support 330 may be rotated about distal-support central longitudinal axis 333 using control handle 340 as described hereinbelow with reference to FIG. 7F.

As shown in the transition between FIG. 5N and FIG. 5O, suture-positioning support 330 is again rotated about distal-support central longitudinal axis 333. For example, suture-positioning support 330 may be rotated about distal-support central longitudinal axis 333 using control handle 340 as described hereinbelow with reference to FIG. 7F. Suture-positioning support 330 is then laterally extended, with respect to distal end portion 332 of elongate support 328, to a fourth deployed position 358, 358D in which suture-positioning support 330 laterally extends in a fourth direction 360D from distal end portion 332 of elongate support 328, fourth direction 360D different from first direction 360A, second direction 360B, and third direction 360C. For example, third direction 360C and fourth direction 360D may be separated by an angle of 120-240 degrees, such as 175-85 degrees, e.g., 180 degrees, around distal-support central longitudinal axis 333 of distal end portion 332 of elongate support 328. For example, suture-positioning support 330 may be laterally extended using control handle 340 as described hereinbelow with reference to FIG. 7G, mutatis mutandis.

As shown in FIG. 5O, while suture-positioning support 330 is in fourth deployed position 358D, second ferrule 324, 324B is proximally withdrawn from ferrule receptacle 334 and out of hollow anatomical structure 54 via a fourth wall site 64D of wall 52, so as to proximally withdraw a portion of second suture 322, 322B, including the distal end portion thereof, out of hollow anatomical structure 54 via fourth wall site 64D. The portion of second suture 322, 322B drawn out of hollow anatomical structure 54 via fourth wall site 64D is secured to another portion of second suture 322, 322D, for example by a second pre-tied knot 338B, such as described hereinbelow with reference to FIGS. 1B-D.

In some applications of the present invention, suturing needles 336 of closure device 320 further comprise a second ferrule-withdrawing suturing needle 336, 336D, which is couplable to second ferrule 324, 324B. As shown in FIG. 5O, closure device 320 is configured to direct second ferrule-withdrawing suturing needle 336, 336D to second ferrule 324, 324B during distal advancement of second ferrule-withdrawing suturing needle 336, 336D. Closure device 320 is configured such that after the distal advancement of second ferrule-withdrawing suturing needle 336, 336D to second ferrule 324, 324B, proximal withdrawal of second ferrule-withdrawing suturing needle 336, 336D coupled to second ferrule 324, 324B removes second ferrule 324, 324B from ferrule receptacle 334, such as shown in FIG. 5P. For example, second ferrule-withdrawing suturing needle 336, 336D may be proximally withdrawn using control handle 340 as described hereinbelow with reference to FIG. 7H regarding first ferrule-withdrawing suturing needle 336, 336B, mutatis mutandis.

For some applications, second ferrule-advancing suturing needle 336, 336C implements any of the features of first ferrule-advancing suturing needle 336, 336A, described hereinabove. For some applications, second ferrule-withdrawing suturing needle 336, 336D implements any of the features of first ferrule-withdrawing suturing needle 336, 336B, described hereinabove. For some applications, second ferrule 324, 324B implements any of the features of first ferrule 324, 324A.

In some applications of the present invention, closure device 320 comprises only a single first suture 322, 322A; a single first ferrule 324, 324A; a single first ferrule-advancing suturing needle 336, 336A; and single second ferrule-withdrawing suturing needle 336, 336B (configuration not shown). In these applications, closure device 320 does not further comprise second ferrule 324, 324B; second suture 322, 322B; second ferrule-advancing suturing needle 336, 336C; or second ferrule-withdrawing suturing needle 336, 336D. In general, all of the features of closure device 320 described herein may implemented with only a single suture, ferrule, ferrule-advancing suturing needle, and ferrule-withdrawing suturing needle.

For some applications, such as shown in FIG. 5P, after second ferrule-withdrawing suturing needle 336, 336D has been proximally withdrawn within control handle 340, second ferrule-withdrawing suturing needle 336, 336D is removed from control handle 340, such as by using a needle handle 350B that is coupled to a proximal end of second ferrule-withdrawing suturing needle 336, 336D. As mentioned above, at this stage of the procedure, second ferrule-withdrawing suturing needle 336, 336D is coupled to second suture 322, 322B via second ferrule 324, 324B. Therefore, removal of second ferrule-withdrawing suturing needle 336, 336D from control handle 340 pulls a portion of second suture 322, 322B out of the control handle. This removal of second ferrule-withdrawing suturing needle 336, 336D from control handle 340 may optionally be implemented as described hereinbelow with reference to FIGS. 7A-H or FIGS. 27A-F for first ferrule-withdrawing suturing needle 336, 336B, mutatis mutandis.

As shown in FIG. 5Q, closure device 320 is withdrawn from blood vessel 54 via puncture 50. In configurations in which dilator 344 is shaped so as to define atraumatic proximal tip 386, the atraumatic proximal tip may facilitate atraumatic maneuvering and passage of dilator 344 through puncture 50. Alternatively, in some configurations in which sheath 410 is provided, sheath 410 may distally advanced along outer tubular shaft 329A so as to cover suture-positioning support 330, dilator connector 368, and proximal end portion 412 of dilator 344, thereby facilitating atraumatic passage of suture-positioning support 330 and/or dilator 344 through puncture 50. Atraumatic proximal tip 386 may help facilitate insertion of proximal end portion 412 of dilator 344 into the distal end of sheath 410.

For some applications, such as shown in FIG. 5R, first suture 322, 322A and second suture 322, 322B are arranged so as to form an X-shaped suturing arrangement, i.e., such that first suture 322, 322A and second suture 322, 322B cross each other. This X-shaped suturing arrangement may provide stronger puncture closure than some alternative suture arrangements, such as arrangements in which a single suture extends around the perimeter of the puncture.

For some applications, such as labeled in FIG. 5R, a distance D between first wall site 64A and second wall site 64B is 5-10 mm, such as 6-8 mm, e.g., 7 mm. For some applications, such as labeled in FIG. 5R, a first line is defined between first wall site 64A and second wall site 64B and a second line is defined between third wall site 64C and fourth wall site 64D. The first and the second lines define an angle α (alpha) of 45-75 degrees, e.g., 60 degrees.

Reference is now made to FIGS. 6A-B, which are schematic illustrations of a configuration of closure device 320, including control handle 340 thereof, in accordance with an application of the present invention.

For some applications, control handle 340 comprises sheath-control user control 430. As shown in the transition between FIG. 6A and FIG. 6B, actuation of sheath-control user control 430 by a user (e.g., by proximal sliding the user control, such as shown), proximally withdraws sheath 410 along outer tubular shaft 329A so as to expose suture-positioning support 330, dilator connector 368, proximal end portion 412 of dilator 344, and distal lumen opening 367. Optionally, sheath-control user control 430 is coupled to sheath 410 by an elongate connector 432. Typically, sheath-control user control 430 is non-electrically activatable. Typically, sheath-control user control 430 is permanently coupled to control handle 340, i.e., not detachable from control handle 340 during ordinary use of control handle 340.

For some applications, sheath-control user control 430 may also be actuated by the user (e.g., by distal sliding the user control), to distally advance sheath 410 along outer tubular shaft 329A so as to cover suture-positioning support 330, dilator connector 368, proximal end portion 412 of dilator 344, and distal lumen opening 367, in order to facilitate atraumatic passage of suture-positioning support 330 and/or dilator 344 through puncture 50, such as described hereinabove with reference to FIG. 5Q.

Reference is still made to FIGS. 6A-B and is further made to FIGS. 7A-H, which are schematic cross-sectional illustrations of a method of using control handle 340 of closure device 320, in accordance with an application of the present invention. (For clarity of illustration, sutures 322 are not shown in FIGS. 7A-H, although they are provided in practice.) For some applications, control handle 340 comprises a support-and-needle user control 434, which may, for example, comprise a button, such as shown. For some applications, support-and-needle user control 434 is configured to be actuated by movement of the user control in a proximal-to-distal direction (such as by depressing the user control, e.g., a button) and/or movement of the user control in a distal-to-proximal direction (such as by releasing the user control, e.g., a button, such that a spring (e.g., a spring 436), moves the user control in the distal-to-proximal direction). For some applications, support-and-needle user control 434 is disposed at a proximal end portion 339 of control handle 340 (labeled in FIGS. 1A, 7A, 25A, 26A, and 34A). Typically, support-and-needle user control 434 is permanently coupled to control handle 340, i.e., not detachable from control handle 340 during ordinary use of control handle 340, including during removal of first and second ferrule-withdrawing suturing needle 336B and 336D from control handle 340. Typically, support-and-needle user control 434 is separate and distinct from needle handle 350A, described hereinabove with reference to FIGS. 5J-K, and from needle handle 350B, described hereinabove with reference to FIG. 5P.

FIG. 7A shows closure device 320 after distal end 406 of outer tubular shaft 329A and suture-positioning support 330 have been inserted through puncture 50 and into blood vessel 54, such as described hereinabove with reference to FIGS. 5A-E, and sheath 410, if provided, has been proximally withdrawn along outer tubular shaft 329A, such as described hereinabove with reference to FIGS. 5D and 6A-B. FIG. 7A shows closure device 320 while (a) support-and-needle user control 434 is in an initial resting state, (b) suture-positioning support 330 is in initial delivery position 356, and (c) first ferrule-advancing suturing needle 336, 336A has not yet been deployed from elongate support 328.

For some applications, such as shown in the transition between FIG. 7A and FIG. 7C, control handle 340 is configured such that actuation of support-and-needle user control 434 (such as by movement of the user control in a proximal-to-distal direction (such as by depressing the user control, e.g., a button)) causes:
lateral extension of suture-positioning support 330, with respect to distal end portion 332 of elongate support 328, to first deployed position 358, 358A in which suture-positioning support 330 laterally extends in first direction 360A from distal end portion 332 of elongate support 328, as shown in FIG. 7B, and such as described hereinabove with reference to FIG. 5F, and
distal advancement of first ferrule-advancing suturing needle 336, 336A, while first ferrule-advancing suturing needle 336, 336A is removably coupled to ferrule 324, such that closure device 320 directs ferrule 324 into ferrule receptacle 334 defined by suture-positioning support 330 and ferrule receptacle 334 removably receives ferrule 324 while suture-positioning support 330 is in first deployed position 358, 358A, as shown in FIG. 7C, and such as described hereinabove with reference to FIG. 5F; typically, this distal advancement of first ferrule-advancing suturing needle 336, 336A distally advances first suture 322, 322A into suture-positioning support 330.

For some applications, control handle 340 is configured such that the actuation of support-and-needle user control 434 causes at least a portion of the distal advancement of first ferrule-advancing suturing needle 336, 336A to occur as suture-positioning support 330 is extended laterally. Optionally, control handle 340 is configured such that the actuation of support-and-needle user control 434 causes suture-positioning support 330 to begin extending laterally before first ferrule-advancing suturing needle 336, 336A begins advancing distally; in this configuration, control handle 340 is typically configured such that the actuation of support-and-needle user control 434 causes suture-positioning support 330 to finish extending laterally, such as shown in FIG. 7B, before first ferrule-advancing suturing needle 336, 336A reaches suture-positioning support 330, such as shown in FIG. 7C. Typically, control handle 340 is configured to additionally provide the timing features described above when extending suture-positioning support 330 and distally advancing first ferrule-withdrawing suturing needle 336B, second ferrule-advancing suturing needle 336C, and second ferrule-withdrawing suturing needle 336D, respectively.

Typically, actuation of support-and-needle user control 434 non-electrically causes the distal advancement of first ferrule-advancing suturing needle 336, 336A and the lateral extension of suture-positioning support 330.

For some applications, such as shown in the transition between FIG. 7A and FIG. 7B, control handle 340 is configured such that the above-mentioned actuation of support-and-needle user control 434 causes the lateral extension of suture-positioning support 330 as follows. Distal motion of support-and-needle user control 434 causes proximal motion of an extension-control-shaft 440 that is coupled to a site 444 on suture-positioning support 330 (labeled in FIGS. 7A-B), the site away from pivot axis 362, i.e., non-coinciding with pivot axis 362. For example, extension-control-shaft 440 may be coupled to site 444 by a rod 442 (which may be either flexible or rigid). The proximal motion of extension-control-shaft 440 pulls up site 444 (labeled in FIGS. 7A-B) on suture-positioning support 330, resulting in rotation of suture-positioning support 330 about pivot axis 362 and lateral extension of suture-positioning support 330. Optionally, extension-control-shaft 440 surrounds at least a portion of inner shaft 329B.

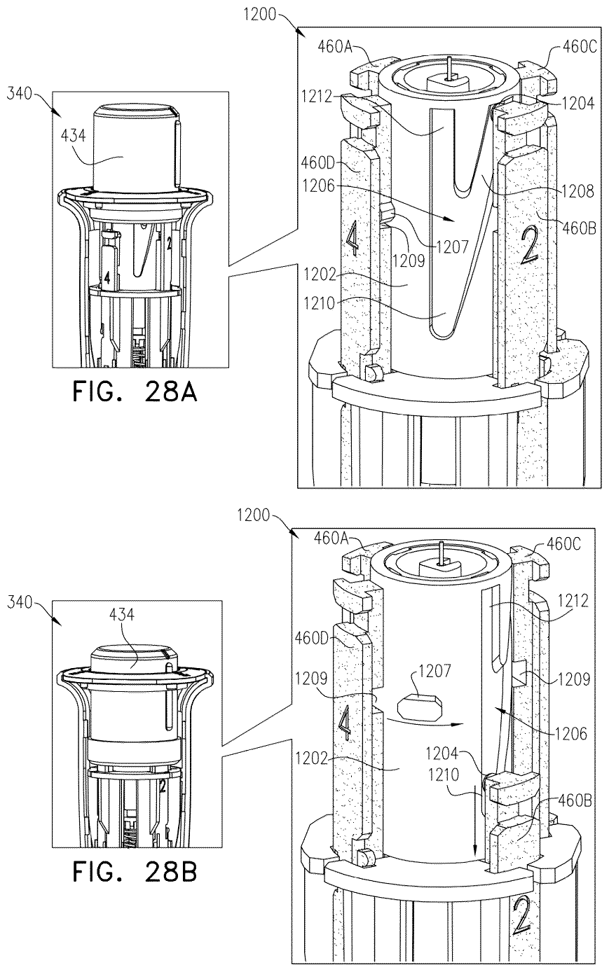

For some applications, control handle 340 comprises a plurality of needle-control shafts 460, including a first needle-control shaft 460A, second needle-control shaft 460B, a third needle-control shaft 460C, and a fourth needle-control shaft 460D (in the cross-sections shown in FIGS. 7A-H, only first and second needle-control shafts 460A and 460B can be seen; the other needle-control shafts 460 are labeled in FIGS. 28A-B, described hereinbelow). Support-and-needle user control 434 engages exactly one of needle-control shafts 460 when support-and-needle user control 434 is in each of its operational rotational positions. Distal motion (depression) of support-and-needle user control 434 distally advances the currently-engaged needle-control shaft 460, thereby laterally extending suture-positioning support 330 and distally advancing the corresponding suturing needle 336, such as described immediately hereinbelow.

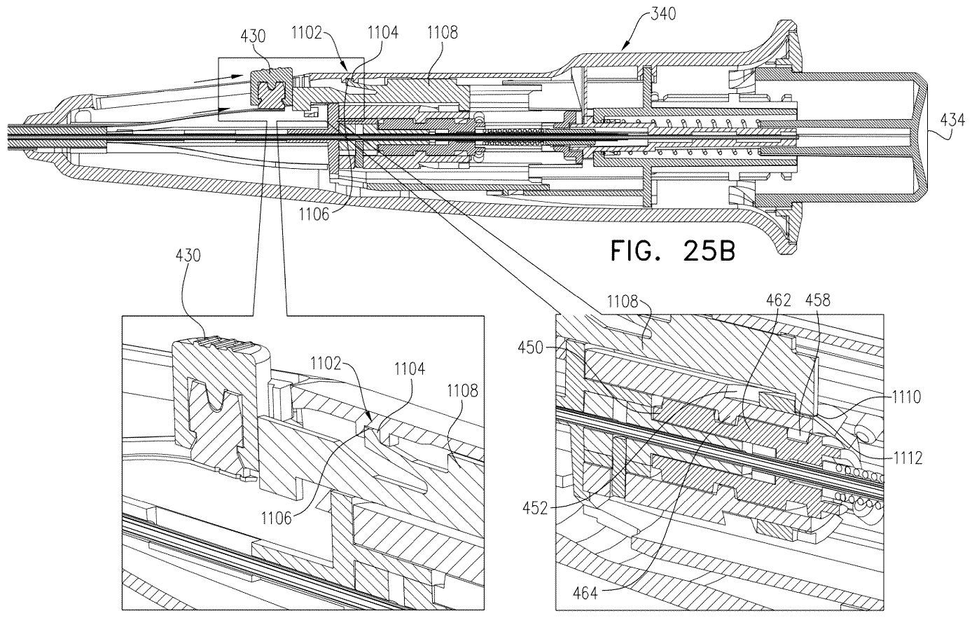

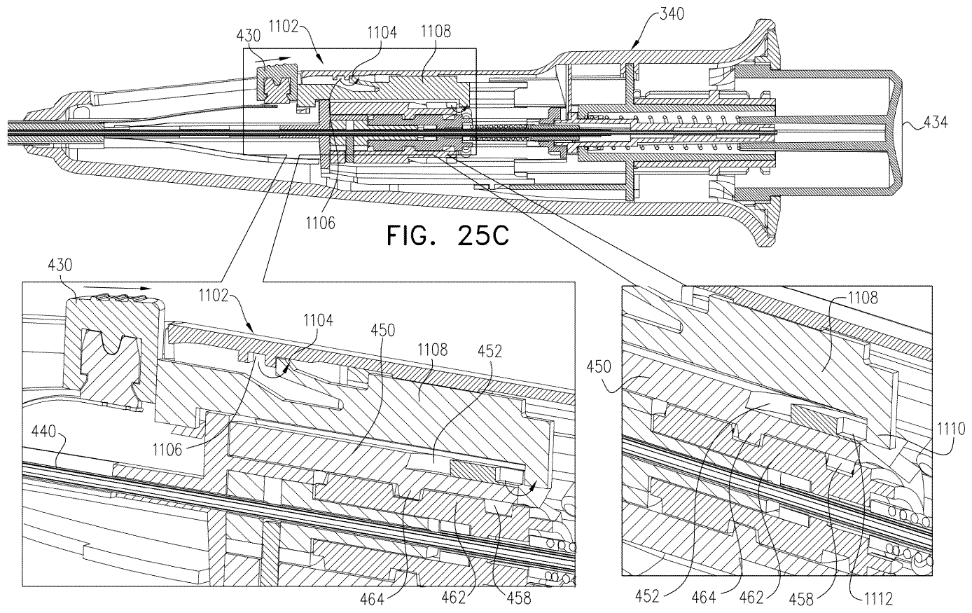

Reference is made to FIGS. 7A-B. For some applications, in order to laterally extend suture-positioning support 330, control handle 340 comprises an outer cam 450 that is shaped so as to define four elongate indentations 452 that include (i) respective curved portions 453 that extend at least partially around a cam axis 454 of outer cam 450 and at least partially axially along outer cam 450, and (ii) respective straight portions 455 that extend axially long outer cam 450. Each of needle-control shafts 460 comprises a pin 456. As each of needle-control shafts 460 is separately distally advanced, as described above, pin 456 of the needle-control shaft 460 engages one of elongate indentations 452, thereby rotating outer cam 450 about cam axis 454 as pin 456 distally advances within curved portion 453 of the elongate indentation 452. Outer cam 450 is shaped so as to define an internal thread 464 (shown and labeled in FIGS. 25A-C, described hereinbelow), which is shaped so as to engage a corresponding external thread 458 defined by an inner cam 462 positioned partially within outer cam 450.

Outer cam 450 is held axially fixed within control handle 340. Inner cam 462 is axially movable within control handle 340. Rotation of outer cam 450 rotates internal thread 464 thereof, which engages external thread 458 of inner cam 462 and causes proximal motion of inner cam 462 with respect to outer cam 450 and control handle 340. Inner cam 462 is coupled to above-described extension-control-shaft 440, such that the proximal motion of inner cam 462 causes the above-described proximal motion of extension-control-shaft 440, and resulting lateral extension of suture-positioning support 330, such as described above.

All of the above-described rotation and motion are reversed as support-and-needle user control 434 is released and moves proximally to its original resting state (such as by spring 436, described hereinabove with reference to FIGS. 7A-H).

For some applications, control handle 340 further comprises:
a cam-stabilizing spring 466, which applies a distally-directed force to outer cam 450 and inner cam 462, to prevent proximal migration of the cams within the control handle; and/or
an inner-cam return spring 468, which applies a distally-directed force to inner cam 462, in order to ensure that inner cam 462 returns proximally to its initial position.

Reference is made to FIGS. 7B-C. As mentioned above, the plurality of needle-control shafts 460 are configured to distally advance respective suturing needles 336. Although the following description relates to first needle-control shaft 460A and first ferrule-advancing suturing needle 336, 336A, the same techniques are implemented for the other needle-control shafts 460 and suturing needles 336, mutatis mutandis. For some applications, in order to distally advance first ferrule-advancing suturing needle 336, 336A, first needle-control shaft 460A is shaped so as to define a distally-facing needle-pushing surface 459. During at least a portion of distal advancement of first needle-control shaft 460A, such as shown in the transition between FIG. 7B and FIG. 7C, distally-facing needle-pushing surface 459 is in contact with and distally pushes a proximal end surface 457 of first ferrule-advancing suturing needle 336, 336A, thereby distally advancing first ferrule-advancing suturing needle 336, 336A.

As described above, in some configurations, control handle 340 is configured such that the actuation of support-and-needle user control 434 causes suture-positioning support 330 to finish extending laterally, such as shown in FIG. 7B, before first ferrule-advancing suturing needle 336, 336A reaches suture-positioning support 330, such as shown in FIG. 7C. For some applications, this relative timing is achieved by the location of distally-facing needle-pushing surface 459 along first needle-control shaft 460A. A first portion of the distal advancement of first needle-control shaft 460A, as shown in the transition between FIG. 7A and FIG. 7B, does not cause distally-facing needle-pushing surface 459 to distally push proximal end surface 457 of first ferrule-advancing suturing needle 336, 336A. A second portion of the distal advancement of first needle-control shaft 460A, as shown in the transition between FIG. 7B and FIG. 7C causes distally-facing needle-pushing surface 459 to distally push proximal end surface 457 of first ferrule-advancing suturing needle 336, 336A. Typically, even though the second portion of the distal advancement of first needle-control shaft 460A causes further distal motion of pin 456, this distal motion does not continue to rotate outer cam 450, because the pin is within the above-mentioned straight portion 455 of elongate indentations 452 during this second portion of the distal advancement of first needle-control shaft 460A.

Typically, support-and-needle user control 434 is partially distally depressed to cause the transition between FIG. 7A and FIG. 7B, and further distally depressed to cause the subsequent transition between FIG. 7B and FIG. 7C.

For some applications, such as shown in the transition between FIG. 7C and FIG. 7E, after the initial actuation of support-and-needle user control 434 to cause the lateral extension and distal advancement of the suturing needle, subsequent actuation of support-and-needle user control 434 (such as by movement of the user control in a distal-to-proximal direction (such as by releasing the user control, e.g., a button)) causes:
proximal withdrawal of first ferrule-advancing suturing needle 336, 336A, a portion of which proximal withdrawal proximally withdraws first ferrule-advancing suturing needle 336 from ferrule 324 while leaving ferrule 324 within ferrule receptacle 334, as shown in FIG. 7D, and such as described hereinabove with reference to FIG. 5G, and
transitioning of suture-positioning support 330 from first deployed position 358, 358A back to delivery position 356, as shown in FIG. 7E, and such as described hereinabove with reference to FIG. 5G.

Proximal motion (release) of support-and-needle user control 434 proximally withdraws the currently-engaged needle-control shaft 460, thereby proximally withdrawing the corresponding suturing needle 336.

For some applications, the subsequent actuation of support-and-needle user control 434 causes at least a portion of the transitioning of suture-positioning support 330 from first deployed position 358, 358A back to delivery position 356 to occur as first ferrule-advancing suturing needle 336, 336A is proximally withdrawn. Optionally, control handle 340 is configured such that the subsequent actuation of support-and-needle user control 434 causes first ferrule-advancing suturing needle 336, 336A to begin proximally withdrawing before suture-positioning support 330 begins transitioning back to delivery position 356. Typically, control handle 340 is configured to additionally provide the timing features described above when transitioning suture-positioning support 330 back to delivery position 356 and proximally withdrawing first ferrule-withdrawing suturing needle 336B, second ferrule-advancing suturing needle 336C, and second ferrule-withdrawing suturing needle 336D.

Reference is made to FIGS. 7C-D. As mentioned above, the plurality of needle-control shafts 460 are configured to proximally withdraw respective suturing needles 336. Although the following description relates to first needle-control shaft 460A and first ferrule-advancing suturing needle 336, 336A, the same techniques are implemented for the other needle-control shafts 460 and suturing needles 336, mutatis mutandis. For some applications, in order to proximally withdraw first ferrule-advancing suturing needle 336, 336A, first needle-control shaft 460A is shaped so as to define a proximally-facing needle-pushing surface 461. During at least a portion of proximal withdrawal of first needle-control shaft 460A, such as shown in the transition between FIG. 7C and FIG. 7D, proximally-facing needle-pushing surface 461 is in contact with and proximally pushes a distally-facing surface 463 of first ferrule-advancing suturing needle 336, 336A, thereby proximally withdrawing first ferrule-advancing suturing needle 336, 336A. For example, a proximal end portion of first ferrule-advancing suturing needle 336, 336A may be bent distally to define distally-facing surface 463, such as shown.

As described above, in some configurations, control handle 340 is configured such that the subsequent actuation of support-and-needle user control 434 causes first ferrule-advancing suturing needle 336, 336A to begin proximally withdrawing, such as shown in FIG. 7D, before suture-positioning support 330 begins transitioning back to delivery position 356, such as shown in FIG. 7E. For some applications, this relative timing is achieved by the location of proximally-facing needle-pushing surface 461 along first needle-control shaft 460A. A first portion of the proximal withdrawal of first needle-control shaft 460A, as shown in the transition between FIG. 7C and FIG. 7D, causes proximally-facing needle-pushing surface 461 to proximally push distally-facing surface 463 of first ferrule-advancing suturing needle 336, 336A. A second portion of the proximal withdrawal of first needle-control shaft 460A, as shown in the transition between FIG. 7D and FIG. 7E causes suture-positioning support 330 to transition back to delivery position 356, and may optionally cause proximally-facing needle-pushing surface 461 to further proximally push distally-facing surface 463 of first ferrule-advancing suturing needle 336, 336A.

Typically, support-and-needle user control 434 is partially proximally released to cause the transition between FIG. 7C and FIG. 7D, and further proximally released to cause the subsequent transition between FIG. 7D and FIG. 7E.

For some applications, as shown in FIG. 7F, actuation of support-and-needle user control 434, such as by rotation of the support-and-needle user control 434 (e.g., comprises a button), rotates suture-positioning support 330 about distal-support central longitudinal axis 333 such as described hereinabove with reference to FIG. 5H. This rotation may be implemented by rotating extension-control-shaft 440 and inner shaft 329B. The rotation may be either clockwise or counterclockwise. FIGS. 7A-E, as well as FIGS. 5E-G, show support-and-needle user control 434 in a first, initial operational rotational position (by way of example, labeled A1 in the figures). FIG. 7F, as well as FIGS. 7G-H and 5H-K, show support-and-needle user control 434 in a second operational rotational position (by way of example, labeled A2 in the figures). FIGS. 5L-N show support-and-needle user control 434 in a third operational rotational position (by way of example, labeled B1 in the figures). FIGS. 5O-P show support-and-needle user control 434 in a fourth operational rotational position (by way of example, labeled B2 in the figures).

For some of these applications, the rotation is achieved by rotation of one or more of the shafts of elongate support 328, with respect to control handle 340, about distal-support central longitudinal axis 333. For example, elongate support 328 may comprise outer tubular shaft 329A and inner shaft 329B, nested within outer tubular shaft 329A, and closure device 320 may be configured to rotate inner shaft 329B while holding outer tubular shaft 329A fixed with respect to control handle 340, such as shown.

For some applications, such as shown in FIG. 7G, actuation of support-and-needle user control 434 (such as by movement of the user control in a proximal-to-distal direction (such as by depressing the user control, e.g., a button)) causes:
lateral extension of suture-positioning support 330, with respect to distal end portion 332 of elongate support 328, to second deployed position 358, 358B in which suture-positioning support 330 laterally extends in second direction 360B from distal end portion 332 of elongate support 328, second direction 360B different from first direction 360A, such as described hereinabove with reference to the transition between FIG. 5H and FIG. 5I, and
distal advancement of first ferrule-withdrawing suturing needle 336, 336B to ferrule 324, which is within ferrule receptacle 334, such as described hereinabove with reference to FIG. 5I.

For some applications, the actuation of support-and-needle user control 434 causes at least a portion of the distal advancement of first ferrule-withdrawing suturing needle 336, 336B to occur as suture-positioning support 330 is extended laterally. For example, the relative timing of the lateral extension of suture-positioning support 330 and the distal advancement of first ferrule-withdrawing suturing needle 336, 336B may be implemented as described hereinabove with reference to FIGS. 7A-C, mutatis mutandis.

For some applications, such as shown in the transition between FIG. 7G and FIG. 7H, subsequent actuation of support-and-needle user control 434 (such as by movement of the user control in a distal-to-proximal direction (such as by releasing the user control, e.g., a button)) causes:
proximal withdrawal of first ferrule-withdrawing suturing needle 336, 336B coupled to ferrule 324, thereby removing ferrule 324 from ferrule receptacle 334, such as described hereinabove with reference to FIG. 5J, and
transitioning of suture-positioning support 330 from second deployed position 358, 358B back to delivery position 356, such as described hereinabove with reference to FIG. 5J.

For some applications, the subsequent actuation of support-and-needle user control 434 causes at least a portion of the transitioning of suture-positioning support 330 from second deployed position 358, 358B back to delivery position 356 as first ferrule-withdrawing suturing needle 336, 336B is proximally withdrawn. The relative timing of the transitioning of suture-positioning support 330 back to delivery position 356 and the proximal withdrawal of first ferrule-withdrawing suturing needle 336, 336B may be implemented as described hereinabove with reference to FIGS. 7C-E, mutatis mutandis.

Typically, control handle 340 of closure device 320 is configured such that support-and-needle user control 434 performs the above functions, mutatis mutandis, for second ferrule-advancing suturing needle 336, 336C and second ferrule-withdrawing suturing needle 336, 336D, such as described hereinabove with reference to FIGS. 5L-P. These functions typically include the rotation of suture-positioning support 330 about distal-support central longitudinal axis 333 such as described hereinabove with reference to FIG. 5L and the transition between FIG. 5N and FIG. 5O.

Reference is made to FIGS. 7A-D. For some applications, distal end 406 of outer tubular shaft 329A is shaped so as to define one or more protrusions 472, such one or more prongs, and suture-positioning support 330 is shaped so as to define a receptacle 474. Insertion of one of protrusions 472 into receptacle 474 helps assure precise angular lateral extension of suture-positioning support 330 with respect to distal end portion 332 of elongate support 328. Alternatively, for some applications, distal end 406 of outer tubular shaft 329A is shaped so as to define one or more receptacles, and suture-positioning support 330 is shaped so as to define a protrusion, such as a prong (configuration not shown). Insertion of the protrusion into one of the receptacles helps assure precise angular lateral extension of suture-positioning support 330 with respect to distal end portion 332 of elongate support 328.

Reference is again made to FIGS. 7A-H. For some applications, needle handle 350A is coupled to the proximal end of first ferrule-withdrawing suturing needle 336, 336B. As shown in FIG. 7H, after first ferrule-withdrawing suturing needle 336, 336B has been proximally withdrawn within control handle 340, first ferrule-withdrawing suturing needle 336, 336B is removed from control handle 340, such as by using needle handle 350A. As mentioned above, at this stage of the procedure, first ferrule-withdrawing suturing needle 336, 336B is coupled to first suture 322, 322A via first ferrule 324, 324A. Therefore, removal of first ferrule-withdrawing suturing needle 336, 336B from control handle 340 pulls a portion of first suture 322, 322A out of the control handle.

For some applications, needle handle 350A and first ferrule-withdrawing suturing needle 336, 336B are removed from control handle 340 via a lateral (side) opening 470 through a wall of control handle 340. For some applications, control handle 340 is configured to prevent removal of needle handle 350A and/or first ferrule-withdrawing suturing needle 336, 336B from control handle 340 until after the proximal withdrawal of first ferrule-withdrawing suturing needle 336, 336B removes first ferrule 324, 324A from ferrule receptacle 334. For some of these applications, such as shown in FIGS. 7A-D, in order to prevent premature passage of needle handle 350A through lateral opening 470, needle handle 350A is initial longitudinally offset from lateral opening 470, such as disposed proximal to lateral opening 470, such as shown in FIGS. 7A-F. This configuration of control handle 340 prevents the premature passage of handle 350A through lateral opening 470 even during distal advancement of first ferrule-withdrawing suturing needle 336, 336B. Typically, lateral opening 470 is disposed distal to proximal end portion 339 of control handle 340.

For some applications (configuration not shown), the needle handles 350 and ferrule-withdrawing suturing needles 336 are removed from control handle 340 via respective proximal openings through a proximal wall of control handle 340 surrounding the proximal opening through the wall through which support-and-needle user control 434 passes.

Typically, one of the actuations of support-and-needle user control 434 longitudinally aligns needle handle 350A with lateral opening 470. For example, the alignment may be caused by the actuation of support-and-needle user control 434 that inter alia causes distal advancement of first ferrule-withdrawing suturing needle 336, 336B, such as shown in the transition between FIGS. 7F and 7G. For example, distal movement of second needle-control shaft 460B may distally move needle handle 350A. (The subsequent proximal movement of second needle-control shaft 460B does not proximally return needle handle 350A to its original longitudinal position, because, for example, needle handle 350A may move radially outward slightly during distal movement of second needle-control shaft 460B, thereby preventing needle handle 350A from returning proximally to its original longitudinal position.)

For some applications, control handle 340 is configured to at least partially eject needle handle 350A from lateral opening 470 after the proximal withdrawal of first ferrule-withdrawing suturing needle 336, 336B removes ferrule 324 from ferrule receptacle 334, such as shown in FIG. 7H. The ejection may occur during or after the proximal withdrawal of first ferrule-withdrawing suturing needle 336, 336B. For example, the wall of control handle 340 that defines a border of lateral opening 470 may be shaped to direct needle handle 350A out of lateral opening 470.

For some applications (configuration not shown), control handle 340 is configured such that support-and-needle user control 434 only causes the distal advancement and proximal withdrawal of suturing needle 336, and not the lateral extension and return of suture-positioning support 330. In these applications, control handle 340 comprises a separate user control for causing the lateral extension and return of suture-positioning support 330. For example, the separate user control may comprise one of user controls 41 described in the '1800 publication with reference to FIGS. 5C and 5I, or user control 141 described in the '1800 publication with reference to FIGS. 7C, 7K, and 8B. In these applications, support-and-needle user control 434 may be considered a needle user control 434. This alternative configuration of control handle 340 may also be optionally implemented in the techniques described hereinbelow with reference to FIGS. 25A-C, 26A-B, 27A-F, and/or 34A-F, mutatis mutandis.

Reference is now made to FIGS. 1B-D. In some applications of the present invention:
first suture 322, 322A is pre-knotted so as to form first pre-tied knot 338A, which is disposed at least partially within closure device 320, such as at least partially within elongate support 328, and/or
second suture 322, 322B is pre-knotted so as to form second pre-tied knot 338B, which is disposed at least partially within closure device 320, such as at least partially within elongate support 328.

Proximal withdrawal of first ferrule-withdrawing suturing needle 336, 336B coupled to first ferrule 324, 324A, such as described hereinabove with reference to FIG. 5J, pulls first ferrule-withdrawing suturing needle 336, 336B and distal end portion 326 of first suture 322, 322A (and first ferrule 324, 324A) through first pre-tied knot 338A. Typically, in order to enable this, first ferrule-withdrawing suturing needle 336, 336B is initially disposed passing through first pre-tied knot 338A.

Proximal withdrawal of second ferrule-withdrawing suturing needle 336, 336D coupled to second ferrule 324, 324B, such as described hereinabove with reference to FIG. 5P, pulls second ferrule-withdrawing suturing needle 336, 336D and second suture 322, 322B (and second ferrule 324, 324B) through second pre-tied knot 338B. Typically, in order to enable this, second ferrule-withdrawing suturing needle 336, 336D is initially disposed passing through second pre-tied knot 338B.

Providing two pre-tied knots 338A and 338B may facilitate formation of the X-shaped suturing arrangement described hereinabove with reference to FIG. 5R.

Optionally, during manufacture of closure device 320, pre-tied knots 338A and 338B are heated while on a mandrel, in order to secure the pre-shaping of the knots. For example, the sutures may comprise a polymer, such as polypropylene, or another material described hereinbelow.

Reference is now made to FIGS. 8A and 8B, which are schematic isometric and cross-sectional views of an alternative configuration of a proximal end portion 512 of a dilator 544, in accordance with an application of the present invention. Sheath 410 is shown distally advanced, such as described hereinabove with reference to FIGS. 5C and 6A.

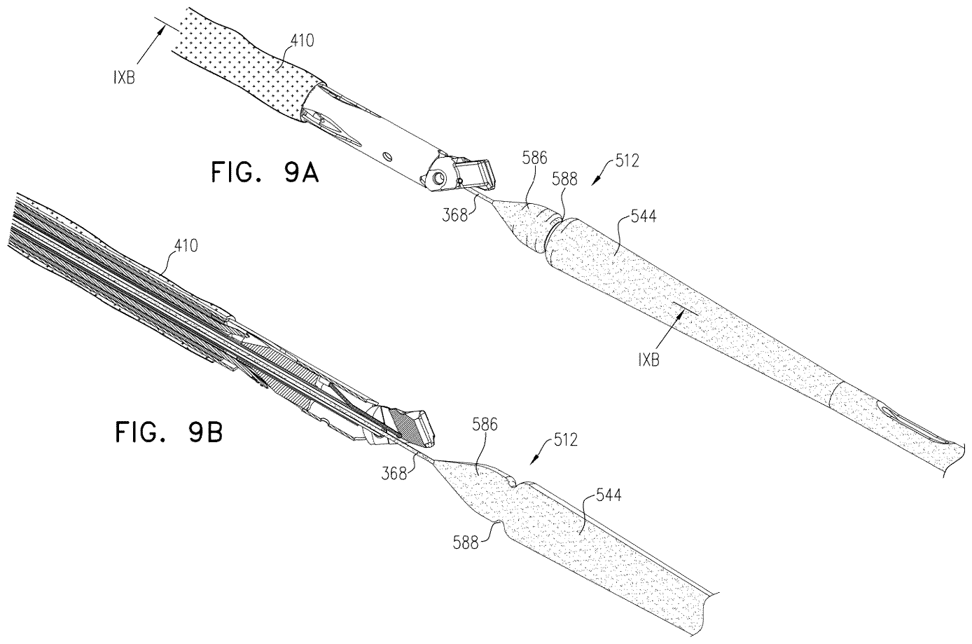

Reference is also made to FIGS. 9A and 9B, which are schematic isometric and cross-sectional views of the alternative configuration of proximal end portion 512 of dilator 544, in accordance with an application of the present invention. Sheath 410 is shown proximally withdrawn, such as described hereinabove with reference to FIGS. 5D and 6B.

Other than as described below, dilator 544 may be identical to dilator 344 described hereinabove, and the features of this configuration may be implemented in combination with any of the features of dilator 344 and/or of closure device 320 described herein, mutatis mutandis.

In this configuration, dilator 544 is shaped so as to define an atraumatic proximal tip 586, which is optionally tapered. Proximal end portion 512 of dilator 544 is shaped so as to define an indentation 588 at least partially around the dilator longitudinally between atraumatic proximal tip 586 and the remaining more distal portion of dilator 544. Indentation 588 may help inhibit (e.g., prevent) distal over-advancement of sheath 410 when sheath 410 is in its distally advanced position, such as shown in FIGS. 8A and 8B and/or during distal advancement of sheath 410, such as described hereinabove with reference to FIG. 5Q.

Reference is now made to FIGS. 10A and 10B, which are schematic cross-sectional illustrations of respective configurations of sheath 410, elongate support 328, and a portion of control handle 340, in accordance with respective applications of the present invention. In these configurations, when sheath 410 is in its distally advanced position, sheath 410, outer tubular shaft 329A of elongate support 328, and a distal portion of control handle 340 are configured to provide a blood flow path 578 between outside sheath 410 and a lateral (side) opening 580 defined by a distal portion of control handle 340. For example, lateral opening 580 may be disposed at an end of a tube 582 that protrudes laterally from control handle 340. The surgeon visually detects blood emerging from lateral opening 580 to indicate that sheath 410, and thus suture-positioning support 330, is disposed within the blood vessel.

Blood flow path 578 may be defined, for example, partially by an interior 590 of sheath 410 and/or partially by one or more tubes 594 disposed within sheath 410, outer tubular shaft 329A of elongate support 328, and the portion of control handle 340.

For some applications, sheath 410 is shaped so as to define one or more openings 592 between outside sheath 410 and interior 590 of sheath 410. For example, the one or more openings 592 may be one or more lateral openings through a wall of sheath 410 (such as shown in FIG. 10-B) and/or one or more distal end openings through the distal end of sheath 410 (configuration not shown).

For some applications, such as shown in FIG. 10B, elongate support 328 further comprises an annular seal 596 that surrounds outer tubular shaft 329A and contacts an inner surface of sheath 410 when sheath 410 is in its distally advanced position, thereby preventing proximal blood flow from interior 590 of sheath 410 into a space between an outer surface of outer tubular shaft 329A and the inner surface of sheath 410, so as to direct the blood flow along blood flow path 578 within outer tubular shaft 329A (e.g., within the one or more tubes 594).

Reference is now made to FIGS. 11A-C, which are schematic illustrations of an alternative configuration of suture-positioning support 330, in accordance with an application of the present invention.

Reference is also made to FIGS. 11D-F, which are schematic illustrations of the alternative configuration of suture-positioning support 330, as well as distal end portion 332 of elongate support 328, elongate flexible dilator connector 368, and a portion of proximal end portion 412 of dilator 344, in accordance with an application of the present invention.

Any of the implementations of suture-positioning support 330 described herein may have the features of the configuration of suture-positioning support 330 shown in FIGS. 11A-F. (By way of example and not limitation, this configuration is also shown in FIGS. 8A-B, 9A-B, and 20A-B.)

In this configuration, an external portion of suture-positioning support 330 that interfaces with distal end portion 332 of elongate support 328 is shaped so as to define one or more partial spherical surfaces 650, such as two partial spherical surfaces 650, as shown in FIGS. 11A-F. These surfaces may help reduce a gap 652 (labeled in FIG. 11E) between the external surface of suture-positioning support 330 and distal end portion 332 of elongate support 328, while still allowing suture-positioning support 330 to pivot between delivery position 356 and the one or more deployed positions 358.

Reference is made to FIG. 11E. For some applications, a length L1 of ferrule 324 is at least 0.8 mm, no more than 2.2 mm, and/or 0.8 mm to 2.2 mm, and/or at least 35%, no more than 100%, and/or 35% - 100%, of a length L2 of a cylindrical portion of ferrule receptacle 334 (excluding an optional funnel-shaped portion of ferrule receptacle 334, if provided). These relative lengths may provide a secure fit for the ferrule within the ferrule receptacle.

Reference is now made to FIGS. 12A-C, which are schematic illustrations of another alternative configuration of suture-positioning support 330, in accordance with an application of the present invention.

Reference is further made to FIGS. 12D-E, which are schematic illustrations of the alternative configuration of suture-positioning support 330 of FIGS. 12A-C, as well as distal end portion 332 of elongate support 328, elongate flexible dilator connector 368, and a portion of proximal end portion 412 of dilator 344, in accordance with an application of the present invention.

Reference is still further made to FIG. 12F, which is a schematic illustration of the alternative configuration of suture-positioning support 330 of FIGS. 11A-F, as well as distal end portion 332 of elongate support 328, elongate flexible dilator connector 368, and a portion of proximal end portion 412 of dilator 344, in accordance with an application of the present invention.

Reference is again made to FIGS. 12A-E (and not to FIG. 12F). Other than as described below, the configuration of suture-positioning support 330 described with reference to FIGS. 12A-E is generally similar to the configuration described hereinabove with reference to FIGS. 11A-F, and may implement any of the features thereof, mutatis mutandis. Any of the implementations of suture-positioning support 330 described herein may have the features of the configuration of suture-positioning support 330 shown in FIGS. 12A-E.

As described hereinabove with reference to FIG. 5G, ferrule receptacle 334 may be shaped so as to define slit 369 through a wall 654 of suture-positioning support 330. In the configuration of FIGS. 12A-F (as well as FIGS. 11A-F and other figures), wall 654 is defined by a free end 656 of suture-positioning support 330 near which ferrule receptacle 334 is disposed.

Unlike in the configuration shown in FIGS. 11A-F (and FIG. 12F, described below), in the configuration shown in FIGS. 12A-E, wall 654 is shaped so as to define a recessed portion 658 along one side 670 of slit 369, thereby enlarging a longitudinal portion of slit 369. For example, recessed portion 658 may define a flat surface that is angled with respect to the remainder of wall 654, such as shown; alternatively, recessed portion 658 is curved, either convexly or concavely (configuration not shown). Recessed portion 658 typically extends along ⅓-⅔ of a length of slit 369.

The enlarged longitudinal portion of slit 369 provided by recessed portion 658 allows the suture to exit ferrule receptacle 334 during rotation of suture-positioning support 330 about distal-support central longitudinal axis 333, for example while suture-positioning support 330 is in delivery position 356, such as described hereinabove with reference to FIGS. 5H and 5L, or while the suture-positioning support is in a deployed position, such as described in the '1800 publication with reference to FIGS. 7A-L or FIGS. 8A-H. The suture exits ferrule receptacle 334 via the enlarged longitudinal portion of slit 369 provided by recessed portion 658, instead via the main opening of ferrule receptacle 334 into which ferrule 324 and suturing needles 336 are inserted. In configurations in which recessed portion 658 is not provided, the suture may instead sometimes exit ferrule receptacle 334 via the main opening of ferrule receptacle 334, which may interfere with the subsequent insertion of a suture needle 336, such as shown in FIG. 12F, which, as described above, illustrates the configuration shown in FIGS. 11A-F.

By way of example and not limitation, FIGS. 12D-F show suture 822 (including distal and proximal suture segments 824A and 824B of suture 822), as described hereinbelow with reference to FIGS. 17A-C. The same principles apply to the other sutures described herein.

For some applications, wall 654 is shaped so as to define two recessed portions 658 respectively along both sides of slit 369 (configuration not shown).

Typically, in configurations in which wall 654 is shaped so as to define is shaped so as to define only one recessed portion 658 along one side 670 of slit 369, the one side 670 is on the trailing side of slit 369 during rotation of suture-positioning support 330 about distal-support central longitudinal axis 333. For example, as shown in FIGS. 12D-E, in order to support counterclockwise rotation of suture-positioning support 330 about distal-support central longitudinal axis 333 (as viewed from the proximal end of elongate support 328), recessed portion 658 is defined by the right side 670 of slit 369 (as viewed from the proximal end of elongate support 328). (The proximal end of elongate support 328 is the end connected to control handle 340.) Similarly, in order to support clockwise rotation of suture-positioning support 330 about distal-support central longitudinal axis 333 (as viewed from the proximal end of elongate support 328), recessed portion 658 is defined by the left side 670 of slit 369 (as viewed from the proximal end of elongate support 328) (configuration not shown).

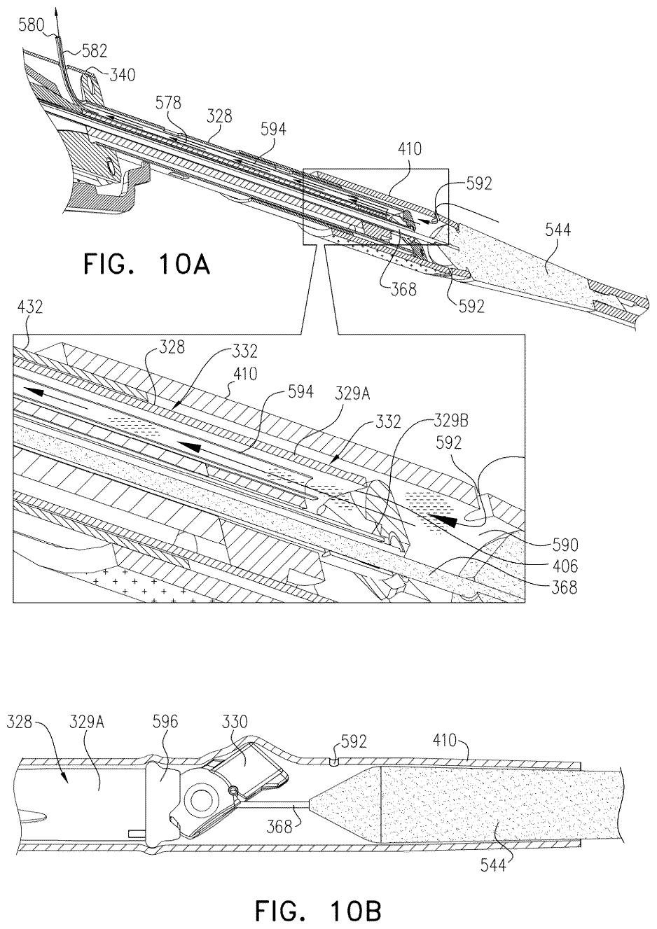

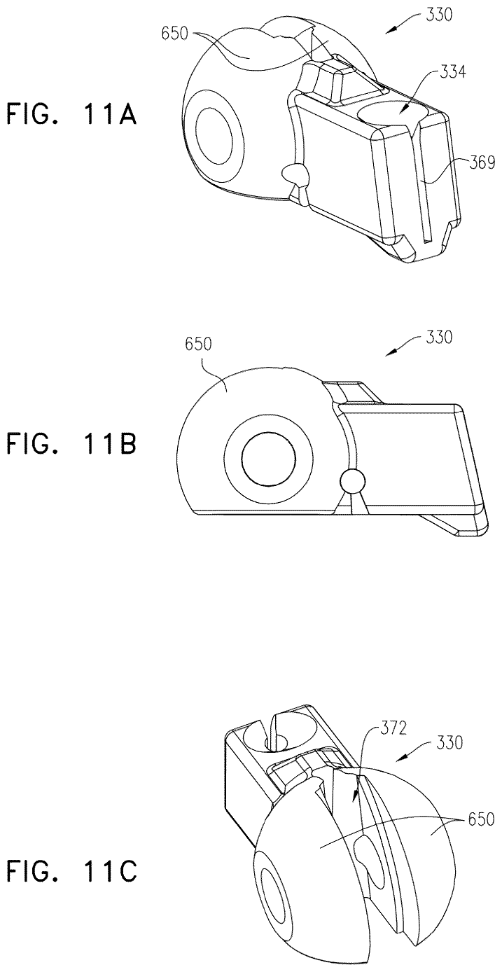

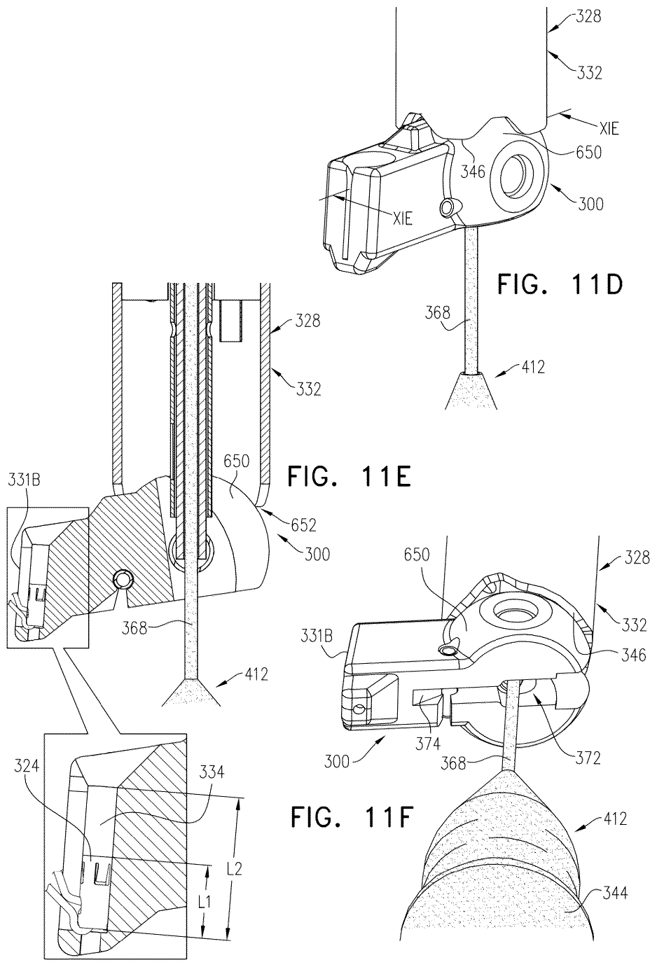

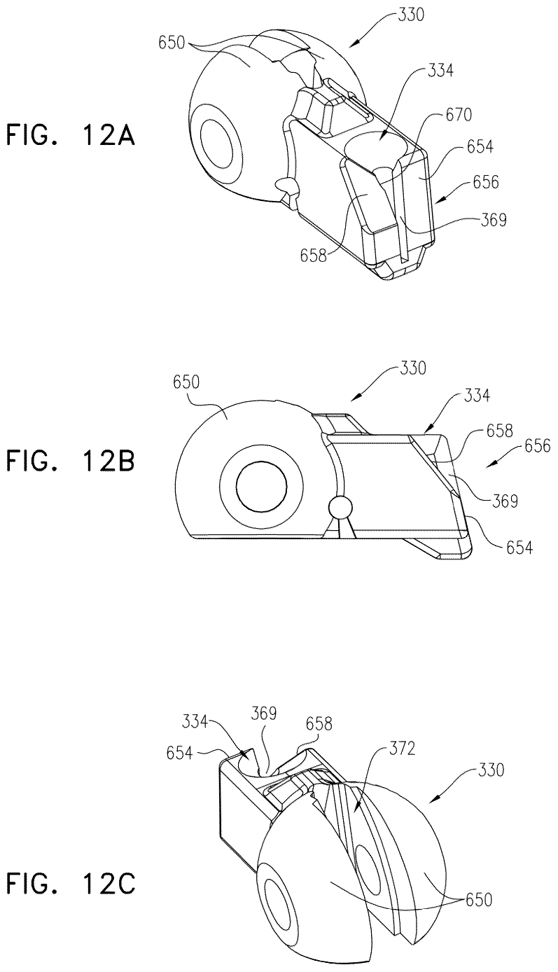

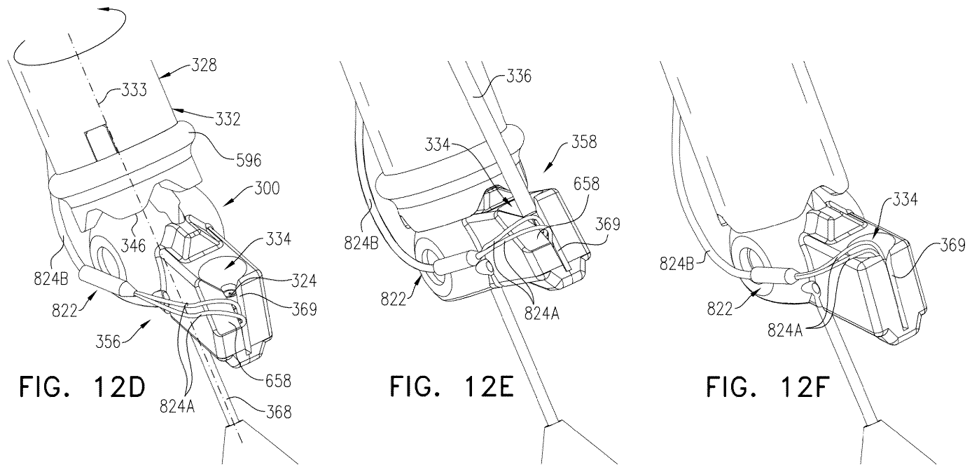

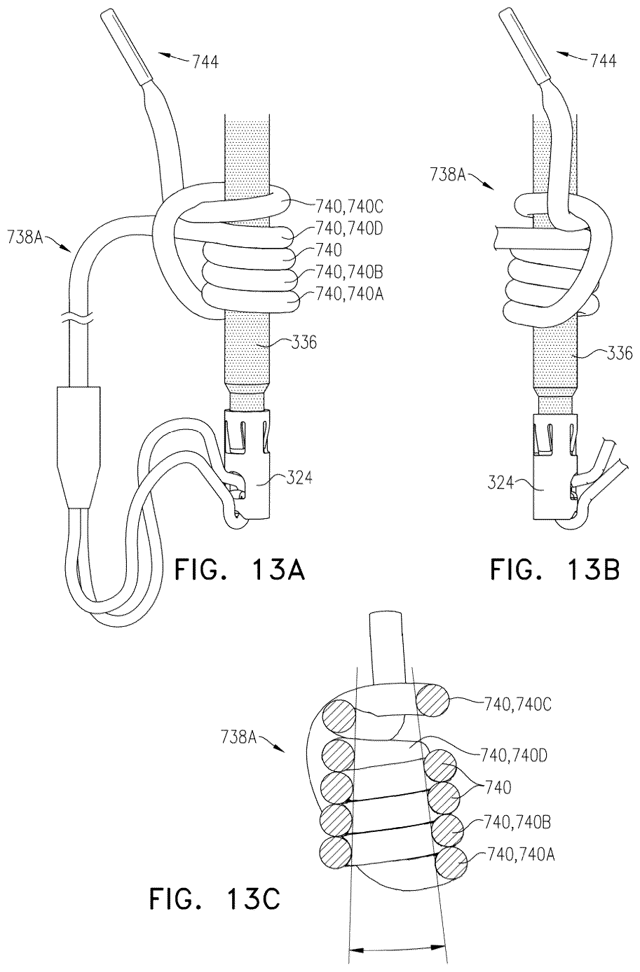

Reference is now made to FIGS. 13A-C and FIGS. 14A-C, which are schematic isometric and cross-sectional illustrations of pre-tied knots 738A and 738B, respectively, in accordance with respective applications of the present invention. Optionally, the features of pre-tied knots 738A and/or 738B are implemented in first pre-tied knot 338A, second pre-tied knot 338B, or both first pre-tied knot 338A and second pre-tied knot 338B, described hereinabove with reference to FIGS. 1B-D. Each of pre-tied knots 738A and 738B are shown with one of the ferrule-withdrawing suturing needles 336, 336B, 336D disposed passing through the knot, such as shown in FIGS. 1B-D, mutatis mutandis.

Each of pre-tied knots 738A and 738B defines a plurality of turns 740, at least two of which have different respective inner diameters. Typically, each of pre-tied knots 738A and 738B defines 4-turns 740 .

As shown in FIGS. 13A-C, an inner diameter of a distal-most turn 740A of pre-tied knot 738A is greater than an inner diameter of a second-to-distal-most turn 740B of pre-tied knot 738A. Distal-most turn 740A is the turn 740 of pre-tied knot 738A located closest to a distal pointed end 742 of ferrule-withdrawing suturing needle 336 (distal pointed end 742 is concealed by ferrule 324 in FIGS. 13A-B, but is labeled in FIG. 1C). For some applications, respective inner diameters of all of turns 740 of pre-tied knot 738A decrease in a distal-to-proximal direction, such as shown. The larger inner diameter of the one or more distal turns 740 may facilitate introduction of ferrule 324 into pre-tied knot 738A as ferrule-withdrawing suturing needle 336 is withdrawn proximally, such as described hereinabove with reference to FIGS. 5H and 5P.

Optionally, during fabrication, pre-tied knot 738A is shaped on a conical mandrel.

As shown in FIGS. 14A-C, an inner diameter of a proximal-most turn 740C of pre-tied knot 738B is greater than an inner diameter of a second-to-proximal-most turn 740D of pre-tied knot 738B. Proximal-most turn 740C is the turn 740 of pre-tied knot 738B located farthest from distal pointed end 742 of ferrule-withdrawing suturing needle 336. For some applications, respective inner diameters of all of turns 740 of pre-tied knot 738B other than proximal-most turn 740C are equal to one another, such as shown; for other applications, respective inner diameters of all of turns 740 of pre-tied knot 738B other than proximal-most turn 740C decrease in a distal-to-proximal direction, such as shown in FIGS. 13A-C. The larger inner diameter of proximal-most turn 740C may compensate for any decrease in the diameter of this loop that may occur during assembly of closure device 320.

For example, the inner diameter of proximal-most turn 740C of pre-tied knot 738B may equal at least 110%, no more than 200%, and/or 110%-200% of the average inner diameter of the other turns 740 of pre-tied knot 738B. By way of example and not limitation, the inner diameter of proximal-most turn 740C of pre-tied knot 738B may be 0.55-0.65 mm, and the average inner diameter of the other turns 740 of pre-tied knot 738B may be 0.65-0.8 mm.

Reference is now made to FIGS. 15A-C and 16A-C, which are schematic illustrations of respective couplings between distal end portion 326 of suture 322 and a ferrule 324A and a ferrule 324B, in accordance with respective applications of the present invention. As described hereinabove with reference to FIGS. 1A-D, 4, and 5F-O, ferrule 324 is coupled to distal end portion 326 of suture 322 (either first suture 322A and/or second suture 322B), such as by being passed (e.g., looped) through an opening defined by a wall of ferrule 324, and/or by welding, knotting, gluing, or another technique (configurations not shown).

In the configurations of ferrule 324, 324A, 324B shown in FIGS. 15A-C and 16A-C, the ferrule is shaped so as to define a blunt interface 750 with distal end portion 326 of suture 322, in order to avoid inadvertent severing of the suture by the ferrule when force is applied to the suture.

For example, in the configuration of ferrule 324, 324A shown in FIGS. 15A-C, a blunt interface 750, 750A is defined by a flap 752 defined by at least a portion of material 756 cut from a wall 754 of ferrule 324, 324A to define an opening 758 through wall 754. The at least a portion of material 756 is bent so as to define flap 752 having a curved bend 760. The at least a portion of material 756 may be bent inward, such as shown in FIGS. 15A-C, or bent outward (configuration not shown). Distal end portion 326 of suture 322 passes through opening 758 (e.g., is looped through opening 758, as shown). When tension is applied to the suture, the distal end portion of the suture contacts curved bend 760 of flap 752, which provides the blunt interface 750, 750A. Alternatively, distal end portion 326 of suture 322 passes through opening 758, without being looped through opening 758, and is instead secured to ferrule 324, 324A by welding, knotting, gluing, or another technique (configuration not shown).

Also for example, in the configuration of ferrule 324, 324B shown in FIGS. 16A-C, a blunt interface 750, 750B is defined by a curved surface 768 of a rod 762 fixed to a perimeter 764 of an opening 758 through a wall 766 of ferrule 324, 324B, such as by welding. Distal end portion 326 of suture 322 passes through opening 758 around rod 762 (e.g., is looped through opening 758 around rod 762, as shown). When tension is applied to the suture, the suture contacts curved surface 768 of rod 762, which provides the blunt interface 750, 750B. Alternatively, distal end portion 326 of suture 322 passes through opening 758 around rod 762, without being looped through opening 758, and is instead secured to ferrule 324, 324B by welding, knotting, gluing, or another technique (configuration not shown).

Reference is now made to FIGS. 17A-C, which are schematic illustrations of a suture 822, in accordance with an application of the present invention. FIG. 17C shows suture 822 after ferrule 324 has been pulled through pre-tied knot 738. Pulling on suture 822 in the direction indicated by the arrows in FIG. 17C tightens the knot. Any of the sutures described herein, including sutures 322, may implement the features of suture 822. Similarly, any of the sutures described herein, including sutures 322 and 822, may optionally comprise a plurality of suture segments coupled in series with one another in any manner, including, but not limited to, using one or more crimps and/or one or more ferrules or other coupling mechanisms.

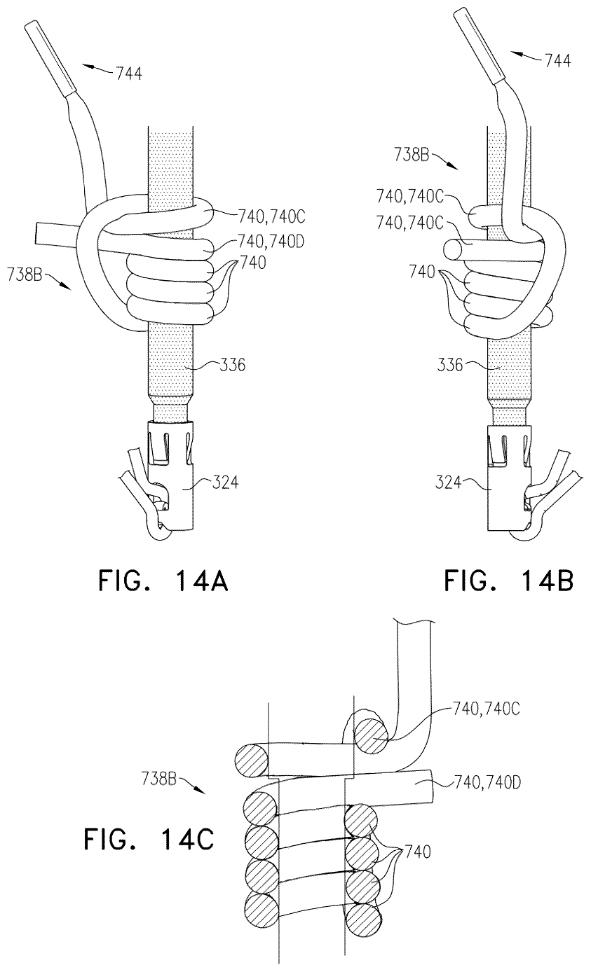

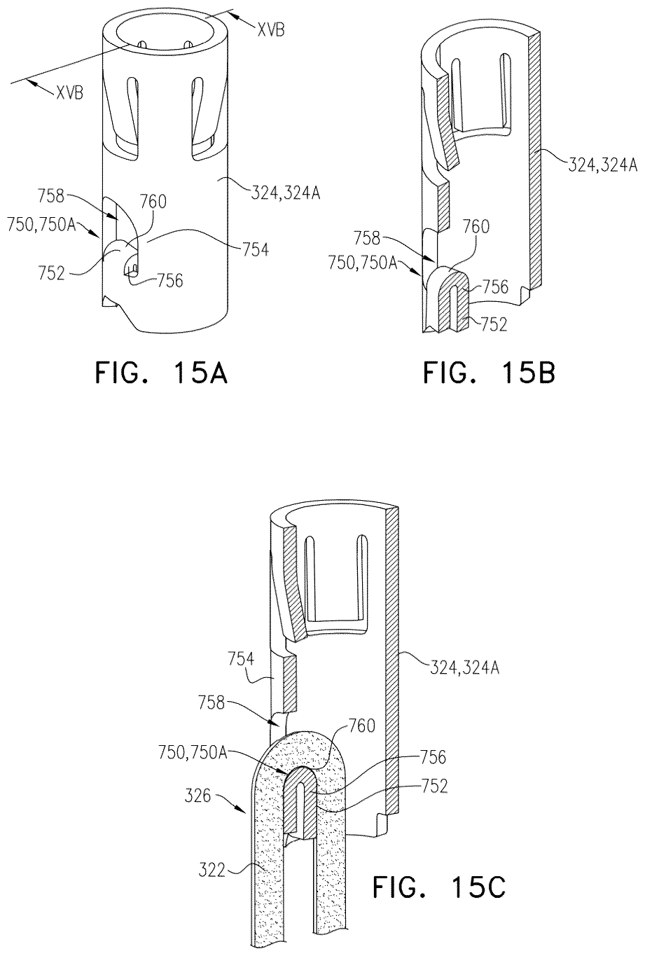

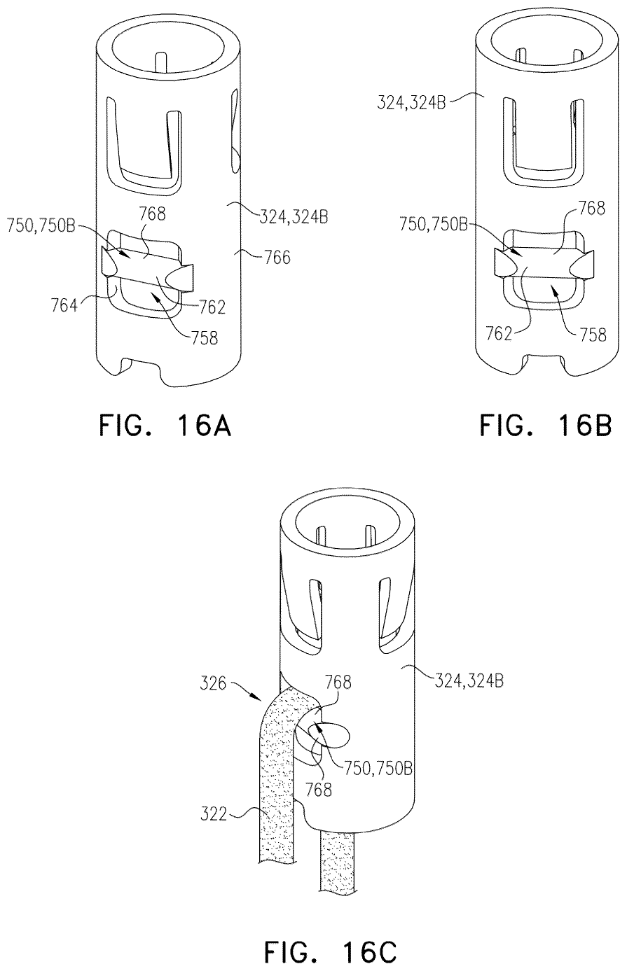

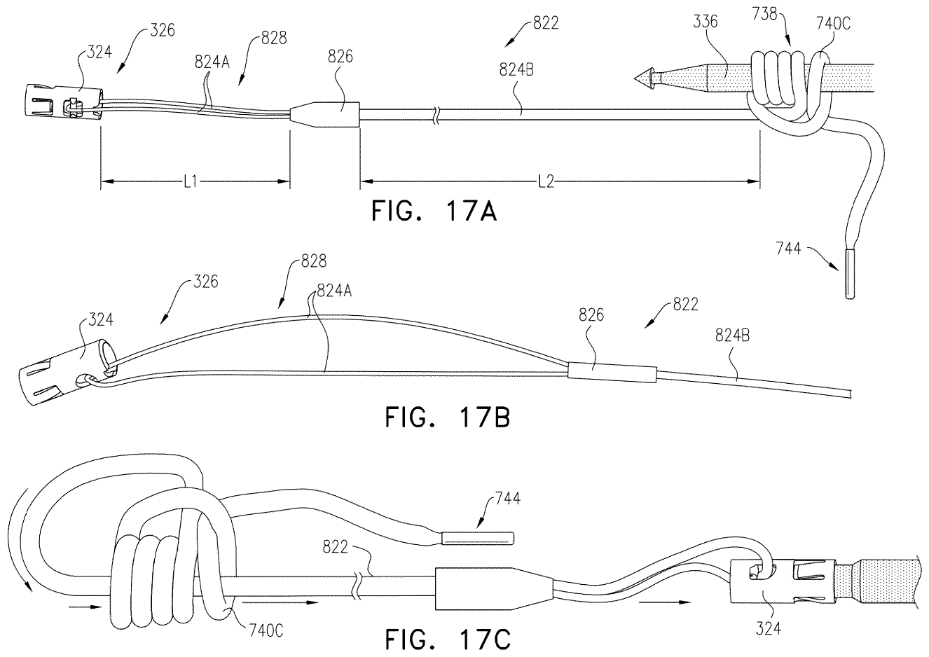

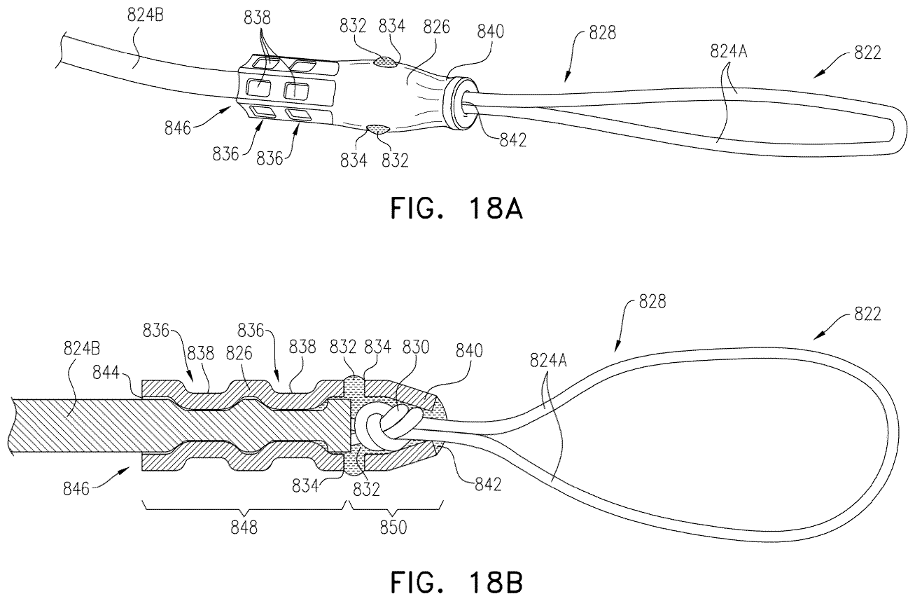

Suture 822 comprises distal and proximal suture segments 824A and 824B, which are non-integral with each other and fixed to each other, for example using a crimp ferrule 826, such as described hereinbelow with reference to FIGS. 18A-B, 19A-B, or 19C-D, or by melting, such as described hereinbelow with reference to FIG. 19E. In configurations in which distal and proximal suture segments 824A and 824B are fixed to each other using a crimp ferrule, the distal and the proximal suture segments may or may not touch each other within crimp ferrule 826. For example, ferrule 324 may be coupled to distal end portion 326 of distal suture segment 824A of suture 822, such as by being passed (e.g., looped) through an opening defined by a wall of ferrule 324, and/or by welding, knotting, gluing, or another technique (configurations not shown).

For some applications, distal suture segment 824A is shaped as a loop 828, such as shown, and the two ends of loop 828 of distal suture segment 824A are fixed to proximal suture segment 824B, such as by being fixed within crimp ferrule 826, e.g., within distal portion 850 of crimp ferrule 826.

Optionally, distal and proximal suture segments 824A and 824B have different diameters, such as shown; for example, a diameter of distal suture segment 824A may be less than a diameter of proximal suture segment 824B. Alternatively, distal and proximal suture segments 824A and 824B may have the same diameter (configuration not shown).

Optionally, distal and proximal suture segments 824A and 824B comprise different types of material; by way of example and not limitation, distal suture segment 824A may comprise PTFE, ePTFE, polypropylene, Nylon, or polyester, and/or proximal suture segment 824B may comprise PTFE, ePTFE, Polypropylene, Nylon, or polyester. Alternatively, distal and proximal suture segments 824A and 824B may comprise the same material, such as one of the materials described immediately above. Any of the sutures described herein may optionally comprise any of these materials. Optionally, distal suture segments 824A, proximal suture segment 824B, and/or any of the other sutures described herein may comprise braided sutures, monofilament sutures, absorbable sutures, or non-absorbable sutures.

For example, distal suture segment 824A may have an effective length L1 of at least 0.5 cm, no more than 2 cm, and/or 0.5-2 cm, and/or proximal suture segment 824B may an effective length L2 of at least 25 cm, no more than 40 cm, and/or 25-40 cm. (In configurations in which a suture segment is shaped as loop 828, such as shown for distal suture segment 824A, the effective length is measured along the doubling of the suture segment, as labeled by L1 in FIG. 17A. The effective lengths do not include portions of suture 822 that are not free, e.g., a portion of proximal suture segment 824B that may be shaped so as to define pre-tied knot 738.)

Reference is made to FIGS. 13A-C and 14A-C. For some applications, proximal-most turn 740C of pre-tied knots 738A and 738B functions to secure (i.e., lock) the pre-tied knot when a free proximal end 744 of the suture that defines turn 740C is pulled tightly. For some applications, free proximal end 744 of proximal-most turn 740C is pre-shaped (such as by heating) in order to ensure that the pre-tied knot is disposed in a relaxed position while positioned within control handle 340. In the absence of this pre-shaping, the suture might have a tendency to return to its original, straight shape, which could affect the position and/or inner diameter of the pre-tied knot. Optionally, free proximal end 744 is marked or comprises a marker to enable easy identification of the free end by the surgeon.

Reference is now made FIGS. 18A-B, 19A-B, and 19C-D, which are schematic isometric and cross-sectional views of respective crimping techniques using crimp ferrule 826, in accordance with respective applications of the present invention.

For some applications, distal and proximal suture segments 824A and 824B comprise the above-mentioned materials.

For example, crimp ferrule 826 may have the following dimensions: 0.5 mm×0.39 mm×2.2 mm.

For some applications, a distal end portion of proximal suture segment 824B is disposed within a proximal portion 848 of crimp ferrule 826 and is fixed to crimp ferrule 826 by crimping, i.e., by making radially-inward crimping indentations 838, such as shown. Optionally, a proximal end portion of distal suture segment 824A is also fixed to crimp ferrule 826 by crimping (configuration not shown); alternatively, the proximal end portion of distal suture segment 824A is fixed to crimp ferrule 826 using one of the techniques described hereinbelow.

For some of these applications, crimping indentations 838 are arranged in two or more axial rows 836 along a longitudinal axis of crimp ferrule 826. Each axial row 836 encircles crimp ferrule 826. For example, each axial row 836 may include two (e.g., three) to ten (e.g., eight) crimping indentations 838. For example, crimping indentations 838 may be arranged in two axial rows 836 having four to six crimping indentations 838 per axial row 836, such as shown in FIGS. 18A-B and 19C-D.

For some applications, the crimping indentations 838 of axially-adjacent axial rows 836 are circumferentially offset (angularly shifted) from one another, such as by 30-90 degrees, e.g., by 30-60 degrees, such as by 45 degrees.

For some applications, the proximal end portion of distal suture segment 824A is disposed within in a distal portion 850 of crimp ferrule 826 (labeled in FIG. 18B).

As labeled in FIGS. 18A-B, distal portion 850 of crimp ferrule 826 may be shaped so as to define a tapered distal tip 840, e.g., a bullet-shaped distal tip, shaped so as to define a distal opening 842 that is smaller than a proximal opening 844 defined by a proximal end 846 of crimp ferrule 826, in order to help hold the proximal end portion of distal suture segment 824A tightly within the crimp ferrule. Typically, during manufacture, distal tip 840 is first tapered and then the proximal end portion of distal suture segment 824A is inserted into distal tip 840.

In the configuration shown in FIGS. 18A-B, the proximal end portion of distal suture segment 824A is knotted into a knot 830 so as to fix the proximal end portion within distal portion 850 of crimp ferrule 826. In configurations in which distal suture segment 824A is shaped as loop 828, the two proximal end portions of loop 828 of distal suture segment 824A are knotted to each other into knot 830, so as to fix the two proximal end portions within distal portion 850 of crimp ferrule 826. Typically, knot 830 is too large to pass through distal opening 842 of crimp ferrule 826. For some applications, an adhesive 832 is disposed at least partially within distal portion 850 of crimp ferrule 826 so as to inhibit unknotting of knot 830. By way of example and not limitation, the knot may comprise a square knot, or a FIG. 8 on a bight knot. Optionally, distal portion 850 of crimp ferrule 826 is shaped so as to define one or more lateral holes 834, and a portion of adhesive 832 protrudes through holes 834 to outside crimp ferrule 826, in order to better secure the distal suture segment with the crimp ferrule. The one or more lateral holes 834 may also be used to introduce adhesive 832 during assembly. For example, each of lateral holes 834 may have a cross-sectional area of 0.007-0.03 mm\hat{ }2. The configuration shown in FIGS. 18A-B may optionally implement either of the indentation options described above with reference to FIGS. 19A-B and 19C-D, respectively.

In the configurations shown in FIGS. 19A-B and 19C-D, the two proximal end portions of loop 828 of distal suture segment 824A are melted together so as to be fixed within distal portion 850 of crimp ferrule 826. Optionally, distal portion 850 of crimp ferrule 826 is shaped so as to define one or more lateral holes 834, and a portion of the melted material protrudes through holes 834 to outside crimp ferrule 826, in order to better secure the distal suture segment with the crimp ferrule.

Reference is now made to FIG. 19E, which is a schematic illustration of another fixation of distal and proximal suture segments 824A and 824B of suture 822, in accordance with an application of the present invention. In this configuration, distal and proximal suture segments 824A and 824B are fixed to each other by melting. Optionally, the two ends of loop 828 of distal suture segment 824A are fixed to the single end of proximal suture segment 824B.

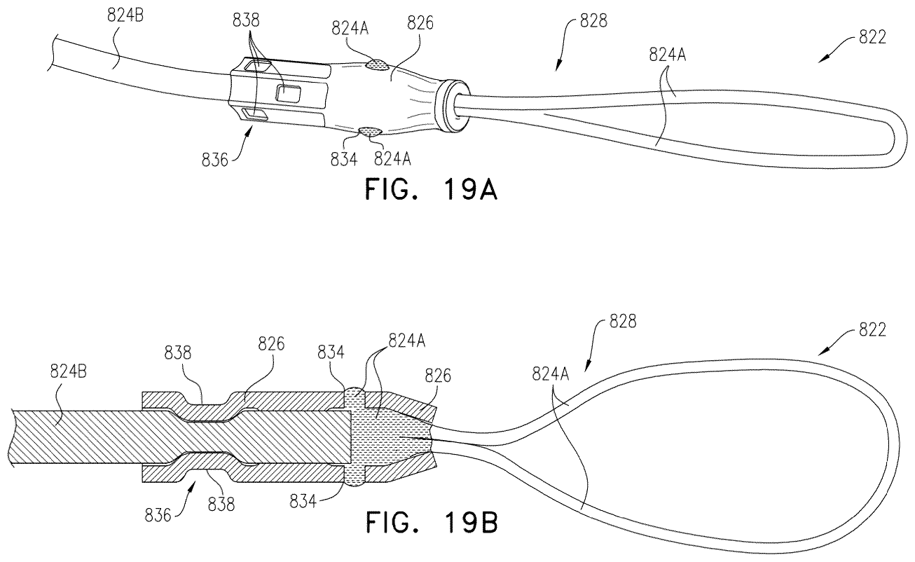

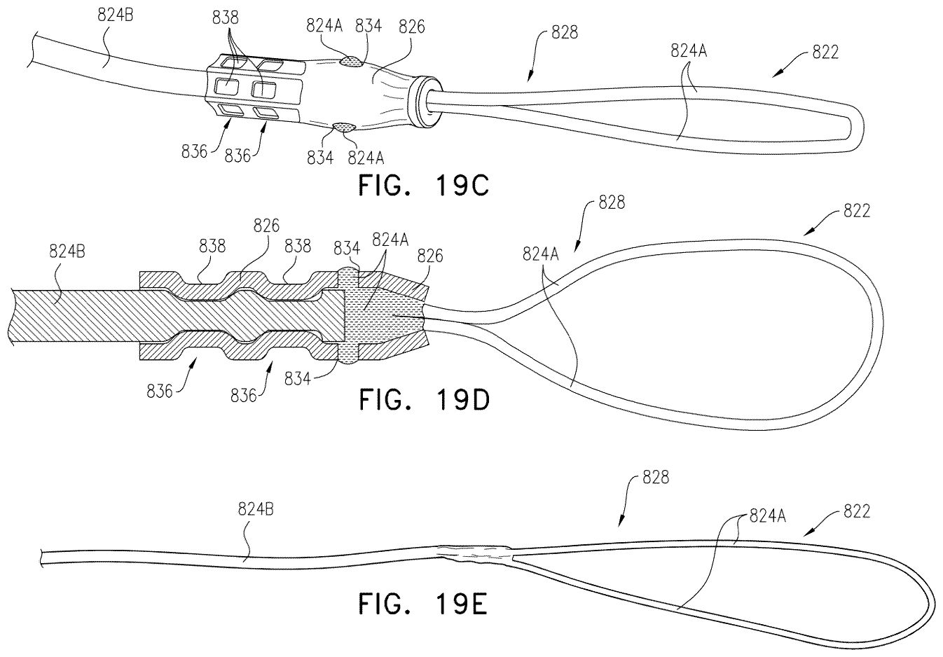

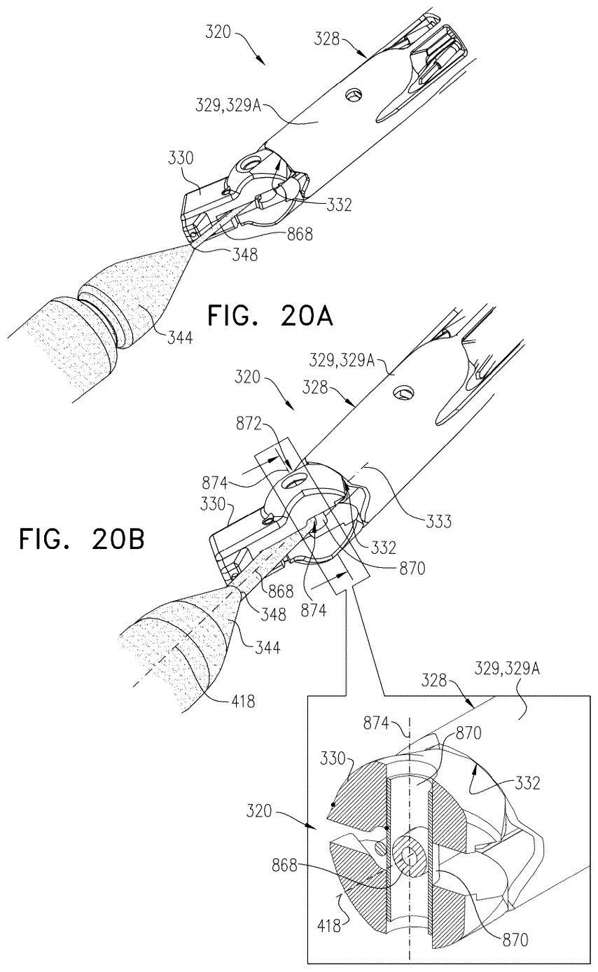

Reference is now made to FIGS. 20A-B, which are schematic illustrations of another configuration of closure device 320, in accordance with an application of the present invention. For clarity of illustration, suture-positioning support 330 is shown as transparent in FIG. 20B.

In the configuration of closure device 320 shown in FIGS. 20A-B, closure device 320 comprises a dilator-connection shaft 868 and a joint 870 that couples dilator-connection shaft 868 to distal end portion 332 of elongate support 328, so as to allow movement of distal end portion 332 of elongate support 328 with respect to proximal end 348 of dilator 344 with at least one rotational degree of freedom. For some applications, dilator-connection shaft 868 and dilator 344 comprise separate respective elements coupled together. Alternatively, dilator-connection shaft 868 and dilator 344 are integrally formed from a single element.

As described hereinabove with reference to FIGS. 1A-4, for some applications, closure device 320 is configured to allow movement of distal end portion 332 of elongate support 328 with respect to proximal end 348 of dilator 344 with at least two degrees of freedom. For some applications, joint 870 that couples dilator-connection shaft 868 to distal end portion 332 of elongate support 328, so as to allow movement of distal end portion 332 of elongate support 328 with respect to proximal end 348 of dilator 344 with at least two degrees of freedom, typically at least two rotational degrees of freedom. For example, joint 870 may comprise:
a hinge 872, which provides a first rotational degree of freedom, such as about an axis 874 perpendicular to distal-support central longitudinal axis 333; and
a twisting joint, which provides a second rotational degree of freedom.

Alternatively, joint 870 may comprise a ball joint, a universal joint, or another joint known in the art.

Joint 870 may be coupled to suture-positioning support 330, such as shown, so as to couple dilator-connection shaft 868 to distal end portion 332 of elongate support 328 via suture-positioning support 330. Alternatively, joint 870 may be directly coupled to distal end portion 332 of elongate support 328.

For example, twisting joint 876 may be coupled to a proximal portion of dilator-connection shaft 868, such as shown, so as to provide the second rotational degree of freedom about distal-support central longitudinal axis 333. Alternatively, twisting joint 876 may couple a distal portion of dilator-connection shaft 868 to proximal end 348 of dilator 344, so as to provide the second rotational degree of freedom about proximal-dilator central longitudinal axis 418 (configuration not shown).

In this configuration, dilator-connection shaft 868 may be flexible or substantially rigid.

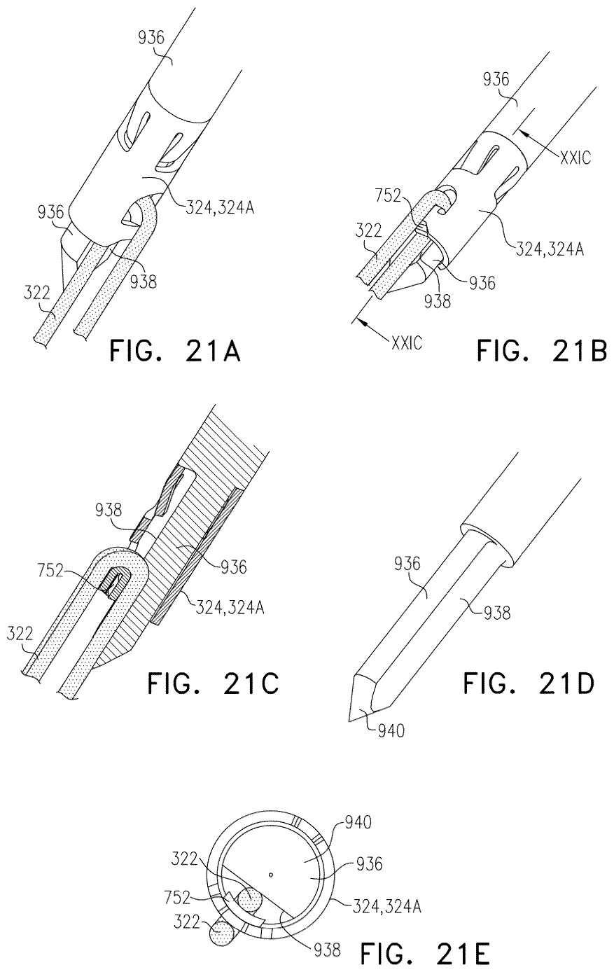

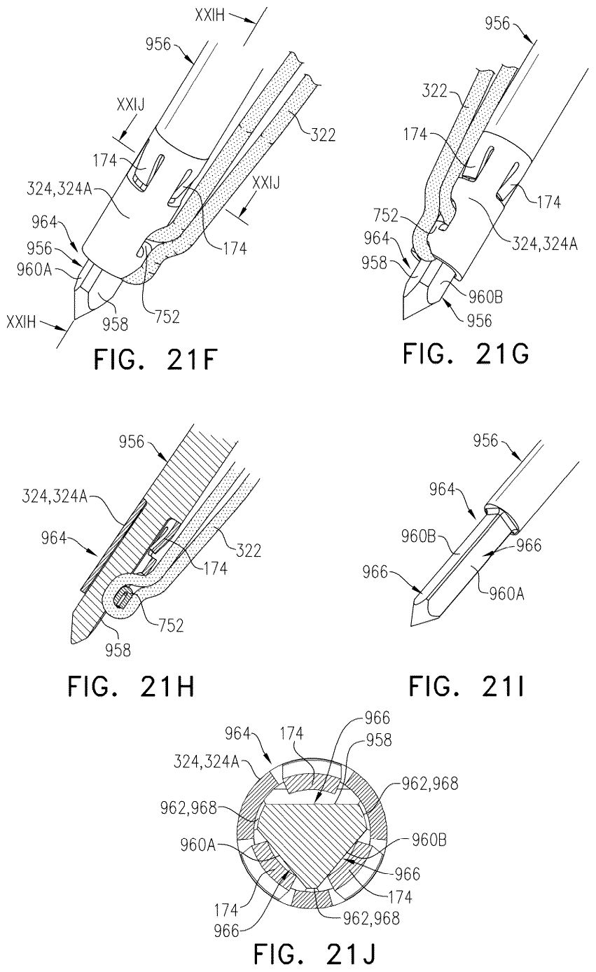

Reference is now made to FIGS. 21A-E, which are schematic illustrations of a ferrule-advancing suturing needle 936, in accordance with an application of the present invention. Optionally, first ferrule-advancing suturing needle 336, 336A and/or second ferrule-advancing suturing needle 336, 336C may implement the configuration of ferrule-advancing suturing needle 936.

A distal end portion of ferrule-advancing suturing needle 936 is shaped and sized so as to be readily insertable into ferrule 324, such as described hereinabove with reference to FIGS. 5F and 5M, while providing space within ferrule 324 for suture 322 alongside a portion of the distal end portion of the needle in configurations in which suture 322 is disposed partially within ferrule lumen 370, such as perhaps best seen in FIGS. 15A-C and 16A-C, as well as FIGS. 21C and 21E. For example, the distal end portion of ferrule-advancing suturing needle 936 may have a flat side 938, such that the distal end portion of the needle may have a generally D-shaped cross section taken perpendicular to a longitudinal axis of the distal end portion of the needle, such as perhaps best seen in FIG. 21E. Typically, a diameter of the distal end portion of needle 936 is less than a diameter of a main proximal portion of needle 936 in order to allow the ferrule to be centered/concentric with the main proximal portion of the needle, while the perpendicular wall (ridge) between the distal end portion and the main proximal portion allows pushing of the ferrule distally.

For applications in which ferrule-advancing suturing needle 936 is used in combination with ferrule 324, 324A, described hereinabove with reference to FIGS. 15A-C, and as shown by way of example and not limitation in FIGS. 21A-E, the size and shape of the distal end portion of ferrule-advancing suturing needle 936 may also provide space within ferrule 324, 324A for flap 752 alongside a portion of the distal end portion of the needle.

Reference is now made to FIGS. 21F-J, which are schematic illustrations of a ferrule-advancing suturing needle 956, in accordance with an application of the present invention. Optionally, first ferrule-advancing suturing needle 336, 336A and/or second ferrule-advancing suturing needle 336, 336C may implement the configuration of ferrule-advancing suturing needle 956.

A distal end portion 964 of ferrule-advancing suturing needle 956 is shaped and sized so as to be readily insertable into ferrule 324, such as described hereinabove with reference to FIGS. 5F and 5M, while optionally providing space within ferrule 324 for suture 322 alongside a portion of the needle, such as perhaps best seen in FIGS. 15A-C and 16A-C, as well as FIGS. 21H and 21J. For example, at least a longitudinal portion of ferrule-advancing suturing needle 956 may have a first flat side 958.

As described hereinabove with reference to FIGS. 5I-J, ferrule 324 is shaped so as to define one or more tabs 373 that are biased to protrude radially inward within ferrule lumen 370 and to engage first or second ferrule-withdrawing suturing needle 336, 336B, 336D upon the insertion of first or second ferrule-withdrawing suturing needle 336, 336B, 336D into a first end opening of ferrule lumen 370, so as to inhibit withdrawal of first or second ferrule-withdrawing suturing needle 336, 336B, 336D from ferrule lumen 370.

Distal end portion 964 of ferrule-advancing suturing needle 956 is also shaped and sized so as both to provide space within ferrule lumen 370 of ferrule 324 for tabs 373, and to contact an internal surface of ferrule lumen 370 so as to grasp ferrule 324. If the ferrule-advancing suturing needle did not provide this space, the needle might expand tabs 373 radially outward. Because tabs 373 typically comprise a plastically-deformable material (such as a metal), the tabs might remain in this radially outward position and thus be less effective in engaging first or second ferrule-withdrawing suturing needle 336, 336B, 336D upon the subsequent insertion of first or second ferrule-withdrawing suturing needle 336, 336B, 336D into ferrule lumen 370, as described above.

For example, distal end portion 964 of ferrule-advancing suturing needle 956 may have second and third flat sides 960A and 960B. The three flat sides 958, 960A, and 960B may provide the needle with a triangular cross section taken perpendicular to a longitudinal axis of the needle (configuration not shown), or a modified triangular cross section in which the flat sides are interspersed with curved (e.g., arcuate) portions 962 around the needle, such as shown (i.e., a triangle with rounded corners). More generally, distal end portion 964 of ferrule-advancing suturing needle 956 may be shaped so as to define two or more elongate indentations 966, which are indented radially inwardly with respect to a geometric cylinder defined by radially-outmost portions 968 of distal end portion 964 of ferrule-advancing suturing needle 956. (Radially-outmost portions 968 define above-mentioned curved portions 962, if provided.) It is to be understood that the geometric cylinder is not an element of the needle, but instead a geometric shape used to define the shape of the needle. Typically, the number of elongate indentations 966 corresponds to the number of tabs 373, or the number of tabs 373 plus one, in order to provide space within ferrule 324 for suture 322 if the suture and one of tabs 373 are not angularly aligned with each other.

For applications in which ferrule-advancing suturing needle 956 is used in combination with ferrule 324, 324A, described hereinabove with reference to FIGS. 15A-C, and as shown by way of example and not limitation in FIGS. 21F-J, the size and shape of ferrule-advancing suturing needle 956 may also provide space within ferrule 324, 324A for flap 752 alongside a portion of distal end portion 964 of the needle.

Alternatively, the flat sides described with reference to FIGS. 21A-E and FIGS. 21F-J are slightly curved, rather than strictly flat.

For some applications, distal end portion 964 of ferrule-advancing suturing needle 956 has a length of at least 0.1 cm, no more than 0.5 cm, and/or 0.1-0.5 cm, and/or a length equal to at least 1.1 times, no more than 4 times, and/or 1.1-4 times a length of ferrule 324, 324A.

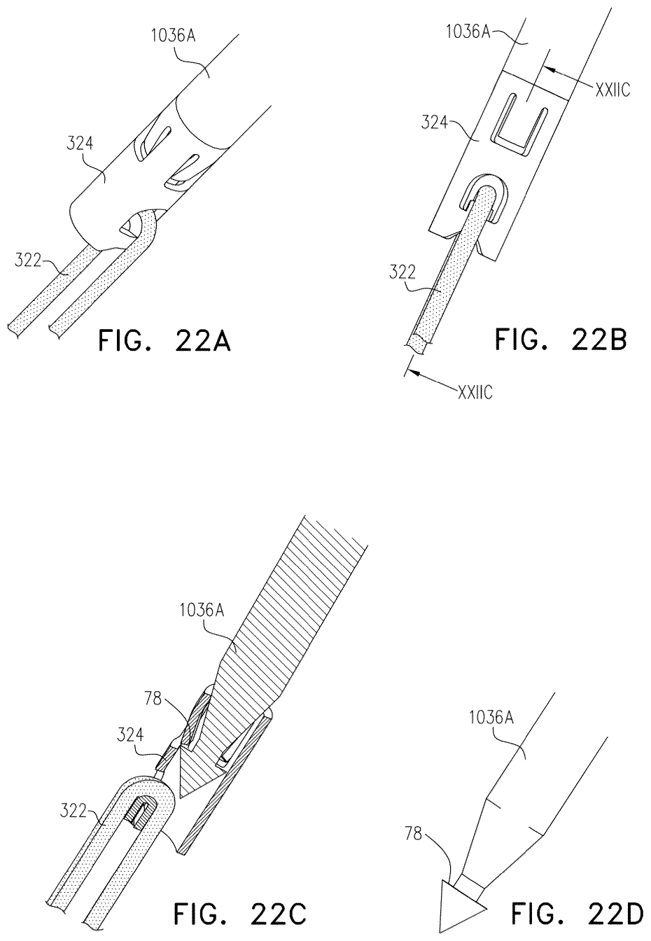

Reference is now made to FIGS. 22A-D, which are schematic illustrations of a ferrule-withdrawing suturing needle 1036A, in accordance with an application of the present invention.

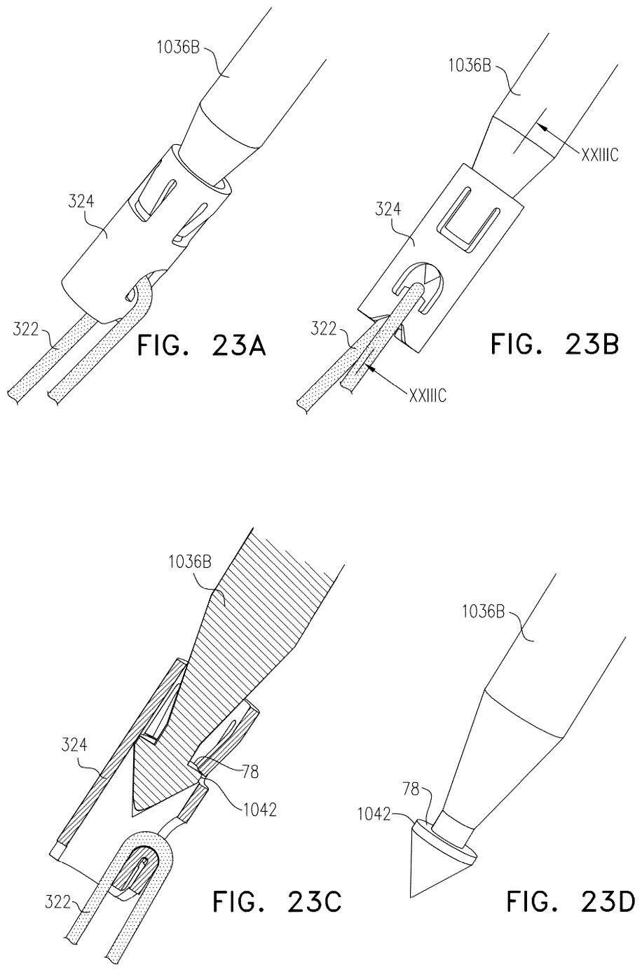

Reference is further made to FIGS. 23A-D, which are schematic illustrations of a ferrule-withdrawing suturing needle 1036B, in accordance with an application of the present invention.

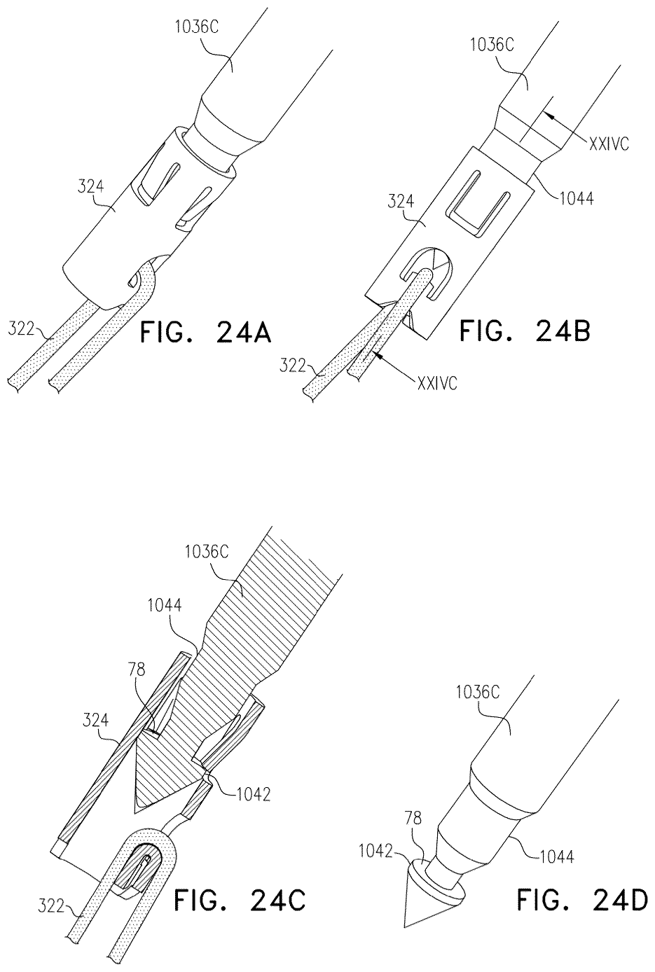

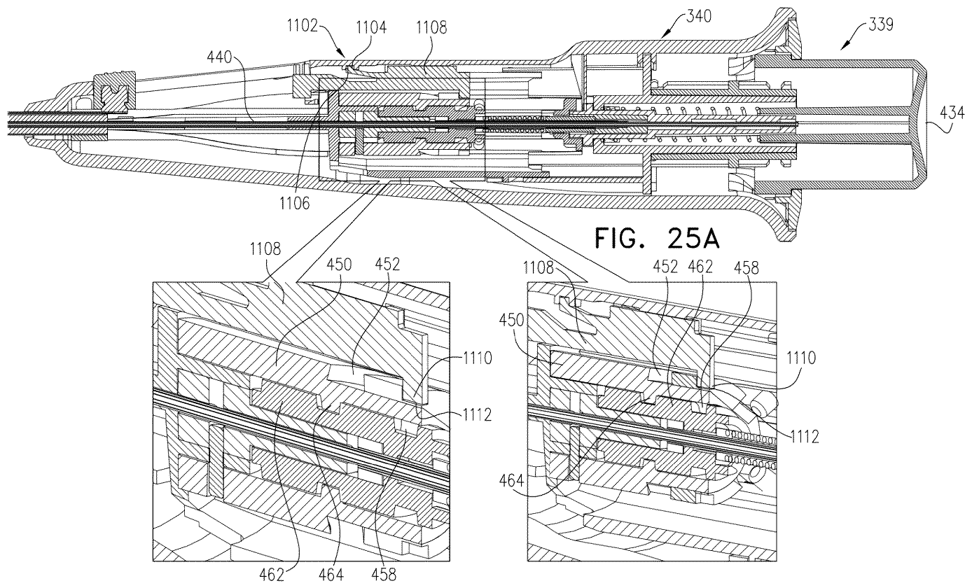

Reference is still further made to FIGS. 24A-D, which are schematic illustrations of a ferrule-withdrawing suturing needle 1036C, in accordance with an application of the present invention.

Optionally, first ferrule-withdrawing suturing needle 336, 336B and/or second ferrule-withdrawing suturing needle 336, 336D may implement the configurations of ferrule-withdrawing suturing needles 1036A, 1036B, and/or 1036C.

Typically, each of ferrule-withdrawing suturing needles 1036A, 1036B, and 1036B is shaped so as to define one or more lateral protrusions 78, which are configured to engage the ferrule upon the insertion of the suturing needle into the ferrule, so as to inhibit withdrawal of the suturing needle from the ferrule.

As shown in FIGS. 23A-D, ferrule-withdrawing suturing needle 1036B may be shaped so as to define a cylindrical portion 1042 immediately distal (i.e., toward the tip of the needle) to the one or more lateral protrusions 78. For example, cylindrical portion 1042 may have a length of 0.05-1 mm. Cylindrical portion 1042 may facilitate consistent manufacturing of ferrule-withdrawing suturing needle 1036B. Optionally, ferrule-withdrawing suturing needle 1036C also is shaped as to define cylindrical portion 1042, as shown in FIGS. 24A-D; alternatively, suturing needle 1036C does not have this feature (configuration not shown, but as shown for suturing needle 1036A).

As shown in FIGS. 24A-D, ferrule-withdrawing suturing needle 1036C is shaped so as to define a cylindrical portion 1044 having a diameter of 80%-99% of an inner diameter of the ferrule and a length of 0.2-1 mm. This shape may reduce an angular degree of freedom of motion between ferrule-withdrawing suturing needle 1036C and the ferrule when at least a portion of cylindrical portion 1044 is inserted into the ferrule.

Reference is now made to FIGS. 25A-C, which are schematic cross-sectional illustrations of a configuration of control handle 340, in accordance with an application of the present invention. Reference is also again made to FIGS. 6A-B. As described hereinabove with reference to FIGS. 6A-B, for some applications, control handle 340 comprises sheath-control user control 430. As shown in the transition between FIG. 6A and FIG. 6B, actuation of sheath-control user control 430 by a user (e.g., by proximal sliding the user control, such as shown), proximally withdraws sheath 410 along outer tubular shaft 329A so as to expose suture-positioning support 330, dilator connector 368, proximal end portion 412 of dilator 344, and distal lumen opening 367 (all labeled in FIG. 6B).

For some applications, control handle 340 is configured to lock support-and-needle user control 434, thereby preventing actuation thereof, such as shown in FIGS. 25A-B, until actuation of sheath-control user control 430, such as shown in FIG. 25C (i.e., while sheath-control user control 430 is a non-actuated state, such as shown in FIGS. 6A and 25A-B). This feature prevents premature lateral extension of suture-positioning support 330 and distal advancement of ferrule-advancing suturing needles 336, 336A and 336, 336C, while sheath 410 would interfere with these operations. This feature may thus serve as a safety mechanism. Actuation of sheath-control user control 430 unlocks support-and-needle user control 434, so as to allow actuation thereof by the user. (FIG. 25B shows sheath-control user control 430 in an intermediate state of activation.) As described above, sheath-control user control 430 is typically mechanically and non-electrically activatable.

As described hereinabove with reference to FIGS. 7A and 7B, rotation of outer cam 450 rotates internal thread 464 thereof, which engages external thread 458 of inner cam 462 and causes proximal motion of inner cam 462 with respect to outer cam 450 and control handle 340. Inner cam 462 is coupled to above-described extension-control-shaft 440, such that the proximal motion of inner cam 462 causes the above-described proximal motion of extension-control-shaft 440, and resulting lateral extension of suture-positioning support 330, such as described above. Each of needle-control shafts 460 comprises a pin 456. As each of needle-control shafts 460 is separately distally advanced, as described above, pin 456 engages one of elongate indentations 452, thereby rotating outer cam 450 about cam axis 454.

For some applications, control handle 340 is configured to lock support-and-needle user control 434 by preventing rotation of outer cam 450. For example, control handle 340 may comprise a key 1110 (which may be considered a pin), and outer cam 450 may be shaped so as to define a keyway 1112 (which may also be considered a groove) shaped to receive key 1110. When sheath-control user control 430 is in a non-actuated state, such as shown in FIGS. 6A and 25A-B, key 1110 is within keyway 1112, thereby preventing rotation of outer cam 450, which in turn prevents lateral extension of suture-positioning support 330 and distal advancement of the currently-engaged needle-control shaft 460, such as described hereinabove with reference to FIGS. 7A-B. This in turn prevents activation of, i.e., locks, support-and-needle user control 434.

For example, control handle 340 may comprise a key-bearing shaft 1108, which comprises key 1110 and is axially moveable with respect to outer cam 450. Actuation of sheath-control user control 430, such as by proximally moving sheath-control user control 430, as shown in FIG. 25C, may move (e.g., push) key-bearing shaft 1108 proximally, thereby removing key 1110 from keyway 1112, and freeing outer cam 450 to rotate upon distal advancement of the currently-engaged needle-control shaft 460. As a result, support-and-needle user control 434 can be activated, i.e., is no longer locked.

For some applications, control handle 340 further comprises a locking interface 1102, which is configured to prevent relocking support-and-needle user control 434 after it has been unlocked. Locking interface 1102 comprises (a) a one-way tab 1104 defined by key-bearing shaft 1108, and (b) a slot 1106. One-way tab 1104 is initially disposed in a slot 1106 when sheath-control user control 430 is the non-actuated state, such as shown in FIGS. 25A-B. The above-described proximal motion of key-bearing shaft 1108 proximally moves one-way tab 1104, thereby removing one-way tab 1104 from slot 1106. Locking interface 1102 is configured to prevent distal movement of one-way tab 1104 after one-way tab 1104 is removed from slot 1106. This prevention of distal movement in turn prevents distal movement of key-bearing shaft 1108, such that key 1110 remains outside keyway 1112, allowing activation of support-and-needle user control 434.

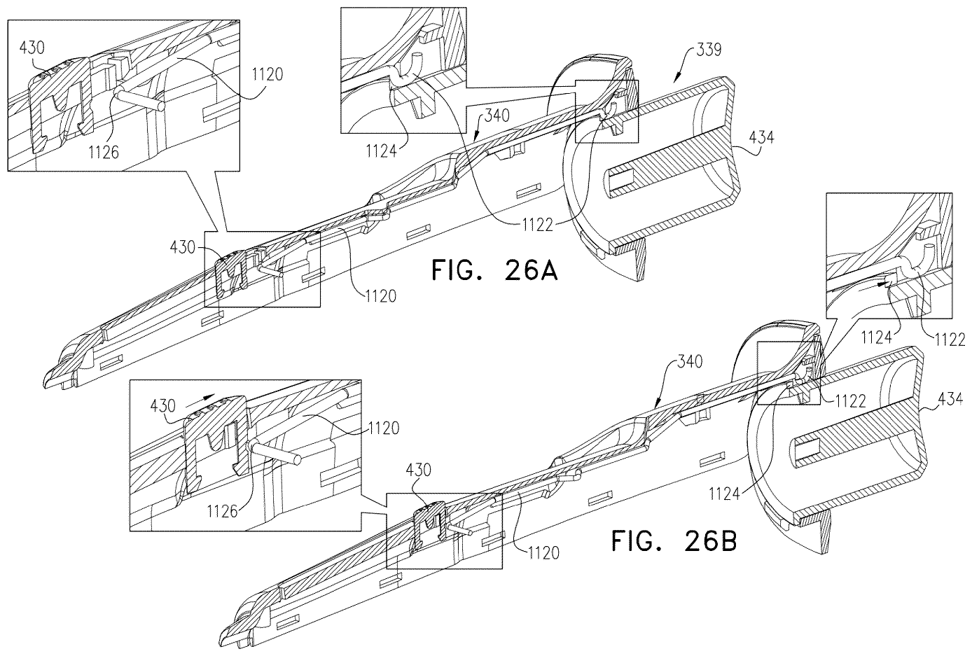

Reference is now made to FIGS. 26A-B, which are schematic cross-sectional illustrations of another configuration of control handle 340, in accordance with an application of the present invention. Reference is also again made to FIGS. 6A-B. As described hereinabove with reference to FIGS. 6A-B, for some applications, control handle 340 comprises sheath-control user control 430. As shown in the transition between FIG. 6A and FIG. 6B, actuation of sheath-control user control 430 by a user (e.g., by proximal sliding the user control, such as shown), proximally withdraws sheath 410 along outer tubular shaft 329A so as to expose suture-positioning support 330, dilator connector 368, proximal end portion 412 of dilator 344, and distal lumen opening 367 (all labeled in FIG. 6B).

For some applications, control handle 340 is configured to lock support-and-needle user control 434, thereby preventing actuation thereof, such as shown in FIG. 26A, until actuation of sheath-control user control 430, such as shown in FIG. 26B (i.e., while sheath-control user control 430 is a non-actuated state, such as shown in FIGS. 6A and 26A). This feature prevents premature lateral extension of suture-positioning support 330 and distal advancement of ferrule-advancing suturing needles 336, 336A and 336, 336C, while sheath 410 would interfere with these operations. This feature may thus serve as a safety mechanism. Actuation of sheath-control user control 430 unlocks support-and-needle user control 434, so as to allow actuation thereof by the user. As described above, sheath-control user control 430 is typically mechanically and non-electrically activatable.

For some applications, control handle 340 is configured to lock support-and-needle user control 434 by preventing rotation of support-and-needle user control 434. For example, control handle 340 may comprise a key-bearing shaft 1120, and a proximal end portion of key-bearing shaft 1120 may define a key 1122 (which may be considered a pin). Support-and-needle user control 434 may be shaped so as to define a keyway 1124 (which may also be considered a groove) shaped to receive key 1122. When sheath-control user control 430 is in a non-actuated state, such as shown in FIGS. 6A and 26A, key 1122 is within keyway 1124, thereby preventing rotation of support-and-needle user control 434 and thus prevents activation of, i.e., locks, support-and-needle user control 434.

Typically, control handle 340 is provided to the user while support-and-needle user control 434 in a locked state. Typically, support-and-needle user control 434 is in a non-operational rotational position, i.e., not one of the operational rotational positions described hereinabove, such as the positions labeled A1, A2, B1, and B2 in FIGS. 5E-P and 7A-H.

Optionally, key-bearing shaft 1120 is not entirely straight, e.g., is shaped to hug close to an internal surface of the wall of control handle 340, such as shown in FIGS. 26A-B.

For some applications, key-bearing shaft 1120 is axially moveable with respect to support-and-needle user control 434, as well as with respect to the wall of control handle 340. Actuation of sheath-control user control 430, such as by proximally moving sheath-control user control 430, as shown in the transition between FIG. 26A and FIG. 26B, may move (e.g., push) key-bearing shaft 1120 proximally, thereby removing key 1122 from keyway 1124, such as by pushing key 1122 proximally beyond keyway 1124. As a result, support-and-needle user control 434 can be activated, i.e., is no longer locked. For example, proximal movement of sheath-control user control 430 may push key-bearing shaft 1120 proximally, e.g., by pushing on a distal end portion 1126 of key-bearing shaft 1120, such as shown.

Reference is again made to FIGS. 6A-B and 7A-H, and is further made to FIGS. 27A-F, which are schematic cross-sectional illustrations of a portion of an alternative control handle 1340 of closure device 320, and a method of using the alternative control handle, in accordance with an application of the present invention. (For clarity of illustration, sutures 322 are not shown in FIGS. 27A-F, although they are provided in practice.) Other than as described below, control handle 1340 is generally similar to control handle 340, described herein, and may implement any of the features thereof, mutatis mutandis. Like reference numerals refer to like parts. The techniques described with reference to FIGS. 6A-B regarding control handle 340 may be applied to control handle 1340, mutatis mutandis.

FIG. 27A shows the portion of control handle 1340 in the state shown in FIG. 7F for control handle 340, in which support-and-needle user control 434 is in the second operational rotational position, after the distal advancement and proximal withdrawal of first ferrule-advancing suturing needle 336, 336A described hereinabove with reference to FIGS. 7A-E.

For some applications, such as shown in FIGS. 27A-B (and FIGS. 7A-D), in order to prevent premature passage of needle handle 350A through lateral opening 470, needle handle 350A is initial longitudinally offset from lateral opening 470, such as disposed proximal to lateral opening 470, as shown in FIG. 27A.

As described hereinabove with reference to FIG. 7G, actuation of support-and-needle user control 434 causes the lateral extension of suture-positioning support 330, and the distal advancement of first ferrule-withdrawing suturing needle 336, 336B to ferrule 324, which is within ferrule receptacle 334.

For some applications, closure device 320 further comprises a needle handle base 1461, which couples needle handle 350A to the proximal end of first ferrule-withdrawing suturing needle 336, 336B. Needle handle base 1461 is shaped so as to define a proximal surface 1463, which faces at least partially proximally. Second needle-control shaft 1460B is shaped so as to define a distal pusher surface 1465, which faces at least partially distally. Distal pusher surface 1465 is shaped so as to engage proximal surface 1463 of needle handle base 1461 upon distal movement of second needle-control shaft 1460B of control handle 1340, such as described hereinabove with reference to FIG. 7G, thereby distally moving needle handle base 1461 and thus needle handle 350A, such as shown in the transition between FIG. 27A and FIG. 27C. Optionally, distal pusher surface 1465 and proximal surface 1463 are angled with respect to a central longitudinal axis of the control handle, such as shown.

FIG. 27A shows control handle 1340 before distal movement of second needle-control shaft 1460B, and thus prior to engagement of proximal surface 1463 of needle handle base 1461 by distal pusher surface 1465.

FIG. 27B shows control handle 1340 immediately upon engagement of proximal surface 1463 of needle handle base 1461 by distal pusher surface 1465 upon the distal movement of second needle-control shaft 1460B.

FIG. 27C shows control handle 1340 after further distal movement of second needle-control shaft 1460B causes distal pusher surface 1465 to distally push proximal surface 1463 of needle handle base 1461, and thus distally advance needle handle 350A. This distal movement also longitudinally aligns needle handle 350A with lateral opening 470.

As described hereinabove with reference to the transition between FIG. 7G and FIG. 7H, subsequent actuation of support-and-needle user control 434 causes proximal withdrawal of first ferrule-withdrawing suturing needle 336, 336B coupled to ferrule 324, thereby removing ferrule 324 from ferrule receptacle 334, and transitioning of suture-positioning support 330 from second deployed position 358, 358B back to delivery position 356.

As shown in the transition between FIGS. 27C and 27E, the subsequent actuation of support-and-needle user control 434 causes proximal withdrawal of first ferrule-withdrawing suturing needle 336, 336B by causing proximal withdrawal of second needle-control shaft 1460B. Second needle-control shaft 1460B is shaped so as to define a proximal surface 1467, which faces at least partially proximally. Needle handle base 1461 is shaped so as to define a distal surface 1469, which faces at least partially distally. The above-mentioned proximal withdrawal of second needle-control shaft 1460B causes proximal surface 1467 of second needle-control shaft 1460B to engage distal surface 1469 of needle handle base 1461 and pull needle handle base 1461 proximally, as shown in the transition between FIG. 27D to FIG. 27F. (This proximal movement of second needle-control shaft 1460B does not proximally return needle handle 350A to its original longitudinal position, because, for example, needle handle 350A may move radially outward slightly during distal movement of second needle-control shaft 1460B, thereby preventing needle handle 350A from returning proximally to its original longitudinal position.)

As shown in FIG. 27F (and FIG. 7H), after first ferrule-withdrawing suturing needle 336, 336B has been proximally withdrawn within control handle 1340, first ferrule-withdrawing suturing needle 336, 336B is removed from control handle 340 via lateral opening 470 through the wall of control handle 1340, using needle handle 350A. As mentioned above, at this stage of the procedure, first ferrule-withdrawing suturing needle 336, 336B is coupled to first suture 322, 322A via first ferrule 324, 324A. Therefore, removal of first ferrule-withdrawing suturing needle 336, 336B from control handle 1340 pulls a portion of first suture 322, 322A out of the control handle.

For some applications, control handle 1340 is configured to at least partially eject needle handle 350A from lateral opening 470 during or after proximal withdrawal of first ferrule-withdrawing suturing needle 336, 336B, such as shown in FIG. 27F. For example, the wall of control handle 340 that defines a border of lateral opening 470 may be shaped to direct needle handle 350A out of lateral opening 470.

Typically, control handle 1340 is configured such that support-and-needle user control 434 performs the above functions, mutatis mutandis, for second ferrule-advancing suturing needle 336, 336C and second ferrule-withdrawing suturing needle 336, 336D, and comprises appropriate elements similar to the elements described above for advancing first ferrule-advancing suture needle 336, 336A and first ferrule-withdrawing suturing needle 336, 336B, mutatis mutandis.

Reference is now made to FIGS. 28A-B, which are schematic illustrations of a needle-advancement safety control assembly 1200, in accordance with an application of the present invention. Control handle 340 may optionally comprise needle-advancement safety control assembly 1200 in combination with any of the other configurations described herein.

Needle-advancement safety control assembly 1200 is configured to prevent distal advancement of second ferrule-advancing suturing needle 336C until first ferrule-withdrawing suturing needle 336B has been distally advanced. This safety feature prevents inadvertent advancement of the suturing needles in the incorrect order.

For some applications, needle-advancement safety control assembly 1200 is configured to prevent distal advancement of second ferrule-advancing suturing needle 336C until first ferrule-withdrawing suturing needle 336B has been distally advanced, by preventing distal advancement of third needle-control shaft 460C (and optionally fourth needle-control shaft 460D), until second needle-control shaft 460B has been distally advanced.

For some of these applications, needle-advancement safety control assembly 1200 comprises a security barrel 1202, an external surface of which is shaped so as to define one or two security pins 1207. Although only one security pin 1207 can be seen in FIGS. 28A-B, a second security pin 1207 is provided on the far side of security barrel 1202.

When needle-advancement safety control assembly 1200 is in a locked state, such as shown in FIG. 28A, security pins 1207 removable engage respective recesses 1209 defined by radially inward surfaces of third needle-control shaft 460C, and optionally fourth needle-control shaft 460D. This engagement prevents axial movement, including distal advancement, of third needle-control shaft 460C, and optionally fourth needle-control shaft 460D.

Rotation of security barrel 1202 transitions needle-advancement safety control assembly 1200 from the locked state shown in FIG. 28A to an unlocked state shown in FIG. 28B. This rotation removes security pins 1207 from the respective recesses 1209, thereby allowing axial movement, including distal advancement, of third needle-control shaft 460C, and optionally fourth needle-control shaft 460D.

Distal advancement of second needle-control shaft 460B causes the above-mentioned rotation of security barrel 1202. For example, the external surface of security barrel 1202 may be shaped so as to define a security track indentation 1206 including a slanted portion 1208 and an axially-aligned portion 1212 that are joined at a distal portion 1210 (e.g., in a quasi-V-like shape). Second needle-control shaft 460B comprises a shaft pin 1204 that extends radially inward and is initially disposed within slanted portion 1208 of security track indentation 1206. The distal advancement of second needle-control shaft 460B distally advances shaft pin 1204 along slanted portion 1208 of security track indentation 1206, which causes the rotation of security barrel 1202. The subsequent proximal movement of second needle-control shaft 460B causes shaft pin 1204 to move proximally within axially-aligned portion 1212 of security track indentation 1206, which does not cause further rotation of security barrel 1202.

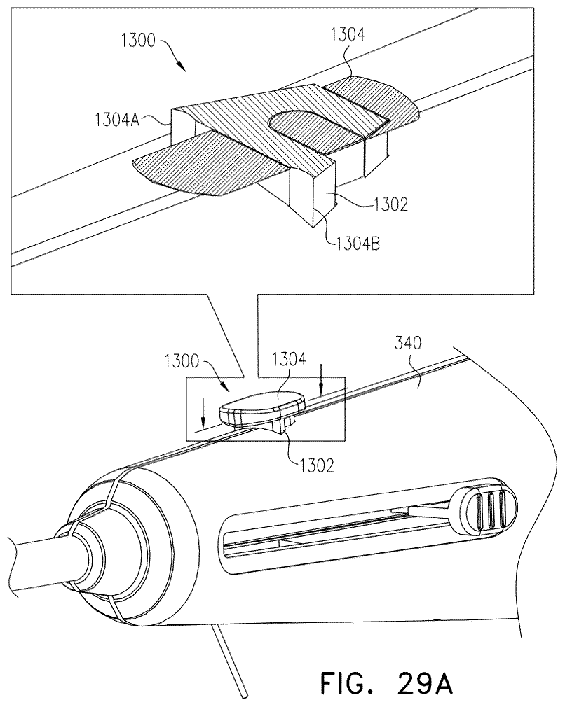

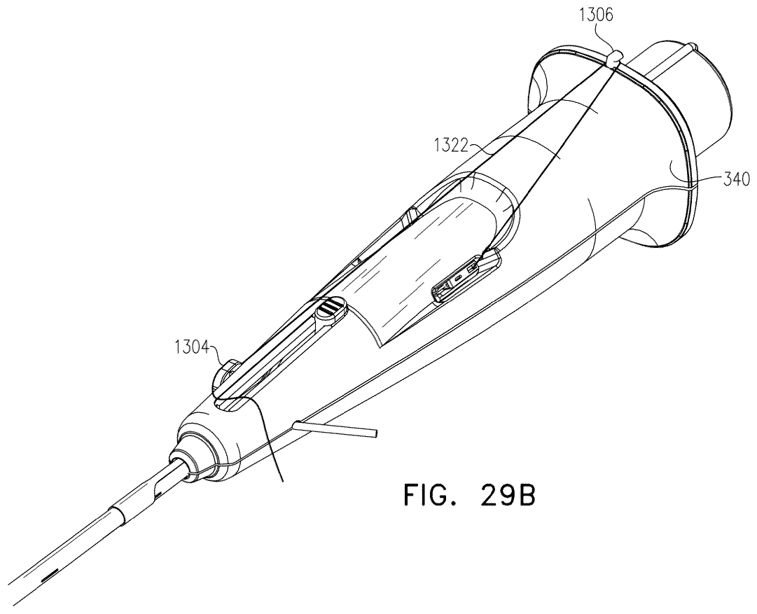

Reference is now made to FIG. 29A, which is a schematic illustration of a cutting blade assembly 1300 of control handle 340, in accordance with an application of the present invention. Cutting blade assembly 1300 is configured to cut the sutures after knotting, as described hereinabove.

Cutting blade assembly 1300 is coupled to an external surface of a housing of control handle 340. Cutting blade assembly 1300 comprises a cutting blade 1302 and a cutting blade cover shield 1304. Cutting blade 1302 is shaped so as to define two sharp edges 1304A and 1304B on opposite sides of cutting blade 1302. Optionally, both sharp edges 1304A and 1304B face at least partially distally. Providing two sharp edges enables the surgeon to cut the suture by pulling it in either direction around control handle 340, such as depending on the handedness of the surgeon or the direction of the knotting.

Reference is made to FIG. 29B, which is a schematic illustration of another configuration of control handle 340, in accordance with an application of the present invention. In this configuration, an external surface of the housing of control handle 340 comprises a suture hook 1306, which facilitates easy engagement of a suture 1332 with cutting blade assembly 1300. Suture 1332 may be any of the sutures described herein. For example, suture hook 1306 may be positioned near a proximal end of control handle 340. Suture hook 1306 may be implemented on the control handles of any of the closure devices described herein or known in the art.

Reference is now made to FIGS. 30A-C, 31A-C, 32A-D, and 33, which are schematic illustrations of a ferrule 1524, a portion of a ferrule-advancing suturing needle 1536, and a portion of a suture 1522, in accordance with respective applications of the present invention. Ferrule 324, described hereinabove, may implement any of the features of ferrule 1524, mutatis mutandis. Ferrule-advancing suturing needles 336, 336A, 336C, described hereinabove, may implement any of the features of ferrule-advancing suturing needle 1536, mutatis mutandis.

Unlike ferrule 324, described hereinabove, ferrule 1524 is shaped so as to define a sharp distal tip 1526. Sharp distal tip 1526 enables advancement of ferrule 1524 through wall 52 of hollow anatomical structure 54 and into blood vessel 54, without the aid of the sharp distal tip of ferrule-advancing suturing needle 336, 336A, 336C. Therefore, in this configuration, ferrule-advancing suturing needle 336, 336A, 336C does not protrude distally beyond the distal end of the ferrule, as shown hereinabove for ferrule 324. Thus, in this configuration, ferrule-advancing suturing needle 336, 336A, 336C may have a blunt distal end 1528, such as shown in FIGS. 30C, 31C, and 32A-D; alternatively, the distal end may have another sharp or blunt shape, such as a semi-spherical or conical shape (configurations not shown).

Sharp distal tip 1526 may have any needle, cannula, or stylet tip shape known in the art, such as a bevel tip, back bevel tip, lancet point tip, or a Franseen tip.

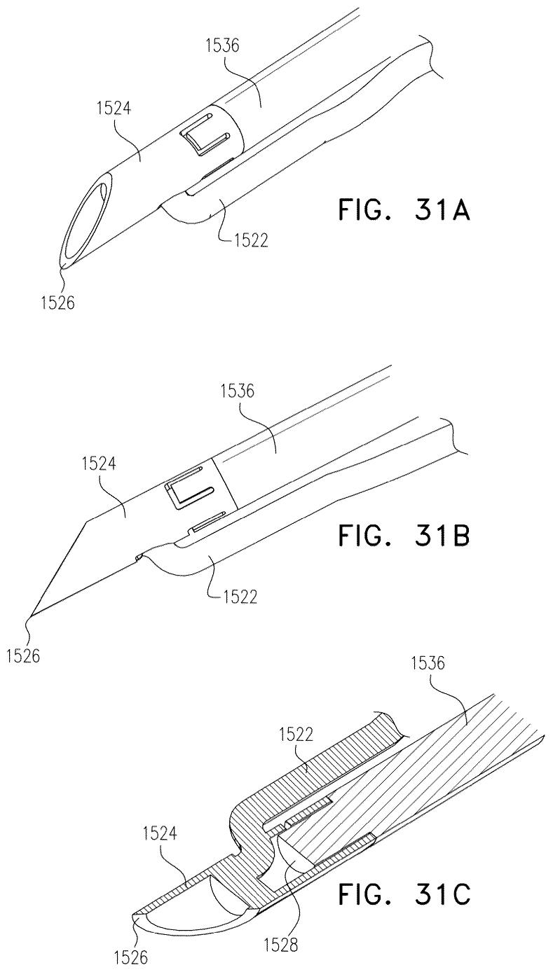

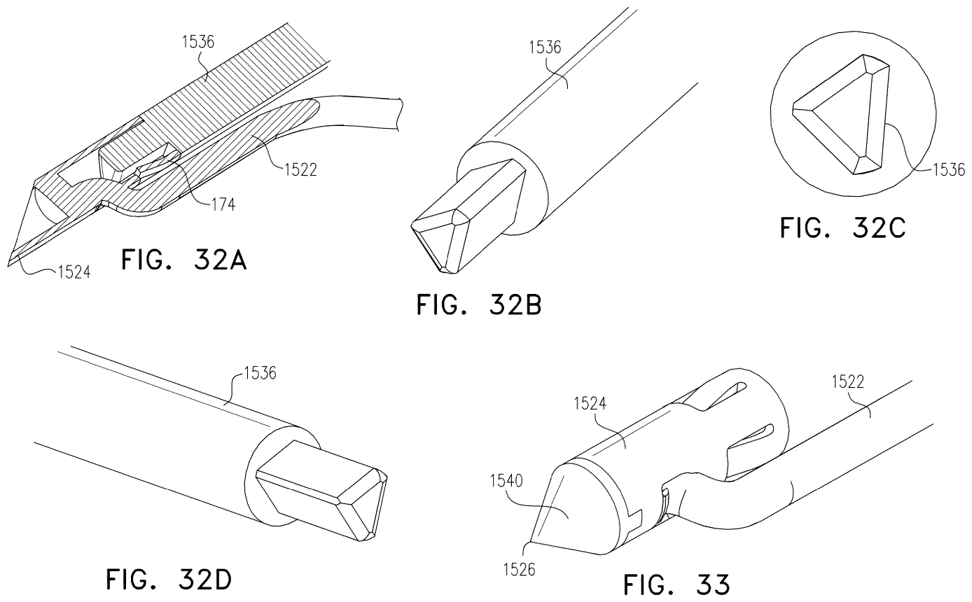

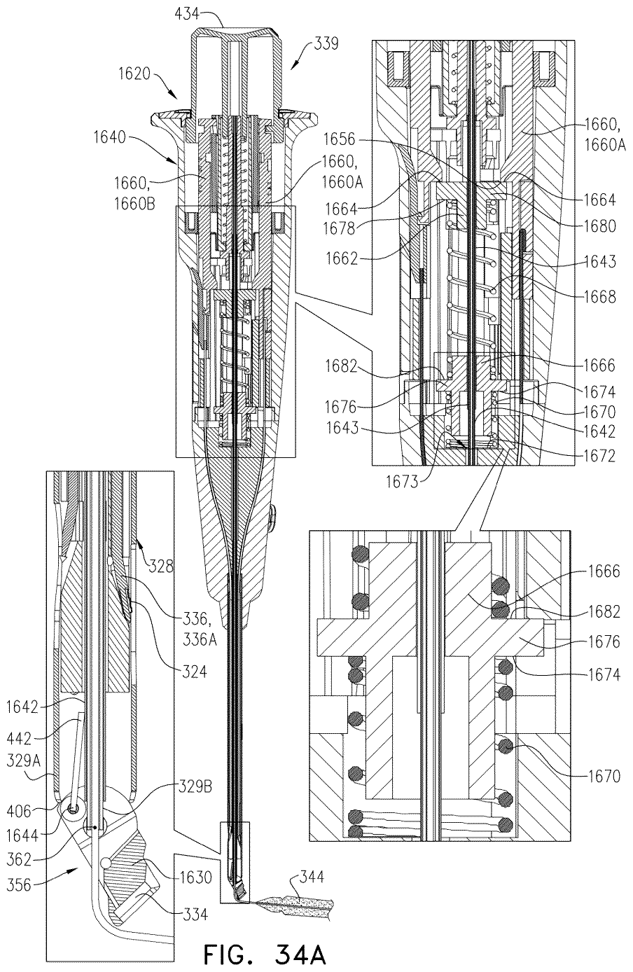

Suture 1522 may implement any of the suture configurations described herein. By way of example and not limitation:
in FIGS. 30A-C, suture 1522 is shown as implementing the suture techniques described hereinabove with reference to FIGS. 17A-C, and
in FIGS. 31A-C, FIGS. 32A-D, and FIG. 33, suture 1522 is shown as being integrated into ferrule 1524; for example, the suture may comprise a polyolefin, such as polypropylene (PP).

In the configuration shown in FIGS. 32A-D, ferrule-advancing suturing needle 1536 implements the triangular or modified triangular shape described hereinabove with reference to FIGS. 21F-J, mutatis mutandis.

In the configurations shown in FIGS. 30A-32D, the portion of ferrule 1524 that is shaped so as to define sharp distal tip 1526 is integral with other portions of ferrule 1524. By contrast, in the configuration shown in FIG. 33, ferrule 1524 comprises a separate distal piece 1540 that is shaped so as to define sharp distal tip 1526, and is coupled to a more proximal portion of the ferrule. In this configuration, the ferrule lumen of ferrule 1524 does not have a second end opening 371B, described hereinabove with reference to FIG. 5F.

Reference is again made to FIGS. 6A-B and 7A-H, and is further made to FIGS. 34A-F, which are schematic cross-sectional illustrations of an alternative control handle 1640 of a closure device 1620, and a method of using the alternative control handle, in accordance with an application of the present invention. (For clarity of illustration, sutures 322 are not shown in FIGS. 34A-F, although they are provided in practice.) Other than as described below, control handle 1640 and closure device 1620 are generally similar to control handle 340 and closure device 1620, respectively, described herein, and may implement any of the features thereof, mutatis mutandis. Like reference numerals refer to like parts. The techniques described with reference to FIGS. 6A-B regarding control handle 340 may optionally be implemented in control handle 1640, mutatis mutandis.

FIG. 34A shows closure device 1620 after distal end 406 of outer tubular shaft 329A and a suture-positioning support 1630 have been inserted through puncture 50 and into blood vessel 54, such as described hereinabove with reference to FIGS. 5A-E, and sheath 410, if provided, has been proximally withdrawn along outer tubular shaft 329A, such as described hereinabove with reference to FIGS. 5D and 6A-B. Other than described below, suture-positioning support 1630 is generally similar to suture-positioning support 330, and may implement any of the features thereof described herein, mutatis mutandis. FIG. 34A shows closure device 1620 while (a) support-and-needle user control 434 is in an initial resting state, (b) suture-positioning support 1630 is in initial delivery position 356, and (c) first ferrule-advancing suturing needle 336, 336A has not yet been deployed from elongate support 328.

For some applications, such as shown in the transition between FIG. 34A and FIG. 34C, control handle 1640 is configured such that actuation of support-and-needle user control 434 (such as by movement of the user control in a proximal-to-distal direction (such as by depressing the user control, e.g., a button)) causes:
lateral extension of suture-positioning support 1630, with respect to distal end portion 332 of elongate support 328, to first deployed position 358, 358A in which suture-positioning support 1630 laterally extends in first direction 360A from distal end portion 332 of elongate support 328, as shown in FIG. 34B, and such as described hereinabove with reference to FIG. 5F, and
distal advancement of first ferrule-advancing suturing needle 336, 336A, while first ferrule-advancing suturing needle 336, 336A is removably coupled to ferrule 324, such that closure device 1620 directs ferrule 324 into ferrule receptacle 334 defined by suture-positioning support 1630 and ferrule receptacle 334 removably receives ferrule 324 while suture-positioning support 1630 is in first deployed position 358, 358A, as shown in FIG. 34C, and such as described hereinabove with reference to FIG. 5F; typically, this distal advancement of first ferrule-advancing suturing needle 336, 336A distally advances first suture 322, 322A into suture-positioning support 1630.

For some applications, control handle 1640 is configured such that the actuation of support-and-needle user control 434 causes suture-positioning support 1630 to finish extending laterally, such as shown in FIG. 34B, before first ferrule-advancing suturing needle 336, 336A reaches suture-positioning support 1630, such as shown in FIG. 34C. For some of these applications, control handle 1640 is configured such that the actuation of support-and-needle user control 434 causes suture-positioning support 1630 to finish extending laterally (as shown in FIG. 34B) before first ferrule-advancing suturing needle 336, 336A begins advancing distally (as shown in FIG. 34C, which also shows the completion of distal advancement of the first ferrule-advancing suturing needle). The relative timing of the lateral extension of suture-positioning support 1630 and the distal advancement of first ferrule-advancing suturing needle 336, 336A is typically based in part on characteristics of the proximal and distal springs 1668 and 1670 (e.g., relative spring constants), described hereinbelow, and/or the initial axial distance between (a) distally-facing needle-pushing surface 1659 of needle-control shaft 1660 and (b) proximal end surface 457 of first ferrule-advancing suturing needle 336, 336A, both described below. Typically, control handle 1640 is configured to additionally provide the timing features described above when extending suture-positioning support 1630 and distally advancing first ferrule-withdrawing suturing needle 336B, second ferrule-advancing suturing needle 336C, and second ferrule-withdrawing suturing needle 336D, respectively.

Typically, actuation of support-and-needle user control 434 non-electrically causes the distal advancement of first ferrule-advancing suturing needle 336, 336A and the lateral extension of suture-positioning support 1630.

For some applications, such as shown in the transition between FIG. 34A and FIG. 34B, control handle 1640 is configured such that the above-mentioned actuation of support-and-needle user control 434 causes the lateral extension of suture-positioning support 1630 as follows. Distal motion of support-and-needle user control 434 causes distal motion of an extension-control-shaft 1642 that is coupled to a site 1644 on suture-positioning support 1630 (labeled in FIGS. 34A-B), the site away from pivot axis 362, i.e., non-coinciding with pivot axis 362, typically such that pivot axis 362 is between site 1644 and the free end of suture-positioning support 1630 near which ferrule receptacle 334 is disposed.

For example, extension-control-shaft 1642 may be coupled to site 1644 by rod 442. The distal motion of extension-control-shaft 1642 pushes down site 1644 (labeled in FIGS. 34A-B) on suture-positioning support 1630, resulting in rotation of suture-positioning support 1630 about pivot axis 362 and lateral extension of suture-positioning support 1630 to deployed position 358, such as shown in FIG. 34B. Optionally, extension-control-shaft 1642 surrounds at least a portion of inner shaft 329B.

For some applications, control handle 1640 comprises a plurality of needle-control shafts 1660, including a first needle-control shaft 1660A, second needle-control shaft 1660B, a third needle-control shaft 1660, and a fourth needle-control shaft 1660 (in the cross-sections shown in FIGS. 34A-F, only first and second needle-control shafts 1660A and 1660B can be seen; the third and fourth needle-control shafts 1660 are similar to needle-control shafts 460C and 460D, labeled in FIGS. 28A-B, described hereinbelow). Support-and-needle user control 434 engages exactly one of needle-control shafts 1660 when support-and-needle user control 434 is in each of its operational rotational positions. Distal motion (depression) of support-and-needle user control 434 distally advances the currently-engaged needle-control shaft 1660, thereby laterally extending suture-positioning support 1630 and distally advancing the corresponding suturing needle 336, such as described immediately hereinbelow.

Reference is made to FIG. 34C. For some applications, first ferrule-advancing suturing needle 336, 336A, when removably coupled to ferrule 324 while ferrule 324 is in ferrule receptacle 334, defines an angle λ (lambda) with distal-support central longitudinal axis 333 of distal end portion 332 of elongate support 328, the angle typically between 0 (i.e., parallel) and 15 degrees, such as between 1 and 14 degrees, e.g., 5-10 degrees, e.g., 6 degrees. Alternatively or additionally, second ferrule-advancing suturing needle 336, 336C; first ferrule-withdrawing suturing needle 336, 336B; and/or second ferrule-withdrawing suturing needle 336, 336D define angle λ with respect to axis 333 when advanced to ferrule receptacle 334.

Reference is made to FIGS. 34A-B. For some applications, in order to laterally extend suture-positioning support 1630, control handle 1640 comprises:
a proximal slider 1662, which is axially moveable within control handle 1640, e.g., by being slidably coupled around extension-control-shaft 1642; proximal slider 1662 is shaped so as to define one or more slider engagement surfaces 1664, e.g., slider proximal engagement surfaces, which face at least partially proximally, as shown;
a distal slider 1666, which is axially moveable within control handle 1640, and axially fixed to extension-control-shaft 1642, e.g., by being coupled around extension-control-shaft 1642;
a proximal spring 1668, which optionally surrounds extension-control-shaft 1642; and
a distal spring 1670.

Proximal spring 1668 is disposed so as to resist movement of proximal slider 1662 and distal slider 1666 toward each other. For example, proximal slider may be disposed partially surrounding proximal slider 1662, partially surrounding distal slider 1666, and partially axially between proximal slider 1662 and distal slider 1666, such as shown.

Control handle 1640 is typically configured such that proximal slider 1662 and distal slider 1666 are rotationally fixed with respect to each other. For example, control handle 1640 may comprise a shaft 1643, which surrounds an axial portion of extension-control-shaft 1642, and is coupled to proximal slider 1662 and distal slider 1666 so as to be axially slidable with respect to proximal slider 1662 and axially fixed with respect to distal slider 1666, but rotationally fixed with respect to both sliders.

Optionally, proximal slider 1662 is shaped so as to define a distally-facing proximal-slider-spring-contact surface 1678, which faces at least partially distally. For example, proximal slider 1662 may be shaped so as to define a proximal-slider flange 1680 that defines distally-facing proximal-slider-spring-contact surface 1678 (optionally, proximal-slider flange 1680 may also be shaped so as to define the one or more slider engagement surfaces 1664 described above, such as shown). Alternatively, a distal end of proximal slider 1662 may be shaped so as to define distally-facing proximal-slider-spring-contact surface 1678 (configuration not shown).

Optionally, distal slider 1666 is shaped so as to define a proximally-facing distal-slider-spring-contact surface 1682, which faces at least partially distally. For example, distal slider 1666 may be shaped so as to define a distal-slider flange 1676 that defines proximally-facing distal-slider-spring-contact surface 1682. Alternatively, a proximal end of distal slider 1666 may be shaped so as to define proximally-facing distal-slider-spring-contact surface 1682 (configuration not shown).

Optionally, proximal spring 1668 is disposed axially between distally-facing proximal-slider-spring-contact surface 1678 and proximally-facing distal-slider-spring-contact surface 1682, contacting both spring-contact surfaces, such as shown.

Control handle 1640 is shaped so as to define an internal proximal stopper surface 1672, which faces at least partially proximally. Optionally, proximal stopper surface 1672 is shaped so as to define an opening 1673 through which extension-control-shaft 1642 passes. Distal slider 1666 is disposed within control handle 1640 axially moveable with respect to proximal stopper surface 1672, so that proximal stopper surface 1672 limits distal advancement of distal slider 1666, i.e., prevents distal slider 1666 from moving distally beyond proximal stopper surface 1672.

Distal spring 1670 is disposed so as to resist the distal advancement of distal slider 1666 toward proximal stopper surface 1672. For example, distal spring 1670 may be disposed partially surrounding distal slider 1666 and partially axially between distal slider 1666 and proximal stopper surface 1672, such as shown. Optionally, distal slider 1666 is shaped so as to define a distally-facing distal-slider-spring-contact surface 1674, which faces at least partially distally, and which engages distal spring 1670. For example, distal slider 1666 may be shaped so as to define above-mentioned distal-slider flange 1676 that defines distally-facing distal-slider-spring-contact surface 1674, such as shown, or a distal end of distal slider 1666 may be shaped so as to define distally-facing distal-slider-spring-contact surface 1674, in which case distal spring 1670 may be disposed entirely axially between distal slider 1666 and proximal stopper surface 1672 (configuration not shown).

Reference is made to FIGS. 34A-C. FIG. 34A shows a resting state, prior to distal advancement of any of the needle-control shafts 1660. In this resting state, proximal spring 1668 and distal spring 1670 are in equilibrium, i.e., they apply equal and opposite forces to distal slider 1666. As mentioned above, the plurality of needle-control shafts 1660 are configured to distally advance respective suturing needles 336. Although the following description relates to first needle-control shaft 1660A and first ferrule-advancing suturing needle 336, 336A, the same techniques are implemented for the other needle-control shafts 1660 and suturing needles 336, mutatis mutandis.

Each of needle-control shafts 1660 comprises a shaft engagement surface 1656, e.g., a distal shaft engagement surface, e.g., shaped as a tab. As each of needle-control shafts 1660 is separately distally advanced, as described above, shaft engagement surface 1656 of the needle-control shaft 1660 engages one or more of the one or more slider engagement surfaces 1664, and distally pushes proximal slider 1662. In turn, proximal slider 1662 applies a distally-directed force to proximal spring 1668.

As shown in the transition between FIG. 34A and FIG. 34B, during a first portion of the distal stroke (distal motion) of needle-control shaft 1660, proximal spring 1668 (a) applies a portion of the distally-directed force to distal slider 1666, and (b) stores the remainder of the distally-directed force as elastic potential energy as the proximal spring axially compresses. The portion of the distally-directed force applied to distal slider 1666 is sufficient to maximally axially advance distal slider 1666 until blocked by proximal stopper surface 1672, as described above. Because distal slider 1666 is axially fixed to extension-control-shaft 1642, as described above, this distal motion of distal slider 1666 distally advances extension-control-shaft 1642, thereby causing the above-described lateral extension of suture-positioning support 1630 to deployed position 358, such as shown in FIG. 34B. Proximal stopper surface 1672 prevents excessive distal advancement of extension-control-shaft 1642.

As shown in the transition between FIGS. 34B and 34C, during a subsequent second portion of the distal stroke of needle-control shaft 1660, a distally-facing needle-pushing surface 1659 of needle-control shaft 1660 comes in contact with and distally pushes proximal end surface 457 of first ferrule-advancing suturing needle 336, 336A, thereby distally advancing first ferrule-advancing suturing needle 336, 336A.

As described above, for some applications, control handle 1640 is configured such that the actuation of support-and-needle user control 434 causes suture-positioning support 1630 to finish extending laterally (as shown in FIG. 34B) before first ferrule-advancing suturing needle 336, 336A begins advancing distally (as shown in FIG. 34C, which also shows the completion of distal advancement of the first ferrule-advancing suturing needle). For some applications, this relative timing is achieved by the location of distally-facing needle-pushing surface 1659 along first needle-control shaft 1660A. The first portion of the distal stroke of needle-control shaft 1660, as shown in the transition between FIG. 34A and FIG. 34B, does not cause distally-facing needle-pushing surface 1659 to distally push proximal end surface 457 of first ferrule-advancing suturing needle 336, 336A. The second portion of the distal stroke of first needle-control shaft 1660A, as shown in the transition between FIG. 34B and FIG. 34C causes distally-facing needle-pushing surface 1659 to distally push proximal end surface 457 of first ferrule-advancing suturing needle 336, 336A.

During the second portion of the distal stroke of first needle-control shaft 1660A, all of the distally-directed force applied to proximal slider 1662 by shaft engagement surface 1656 of the needle-control shaft 1660 is stored as elastic potential energy as proximal spring 1668 further axially compresses.

Typically, support-and-needle user control 434 is partially distally depressed to cause the transition between FIG. 34A and FIG. 34B, and further distally depressed to cause the subsequent transition between FIG. 34B and FIG. 34C.

It is noted that control handle 1640 utilizes only linear movement of the elements thereof to cause suture-positioning support 1630 to extend and return laterally. This differs from control handle 340, which also uses rotational motion (of outer cam 450) to cause suture-positioning support 330 to extend and return laterally.

For some applications, such as shown in the transition between FIG. 34C and FIG. 34F, after the initial actuation of support-and-needle user control 434 to cause the lateral extension and distal advancement of the suturing needle, subsequent actuation of support-and-needle user control 434 (such as by movement of the user control in a distal-to-proximal direction (such as by releasing the user control, e.g., a button)) causes proximal withdrawal of first ferrule-advancing suturing needle 336, 336A. Typically, the elastic potential energy earlier stored in proximal spring 1668 and distal spring 1670, as described above, causes this movement of the user control in a distal-to-proximal direction upon release of the user control.

As shown in the transition between FIGS. 34C and 34E, a first portion of the proximal withdrawal proximally withdraws first ferrule-advancing suturing needle 336, 336A from ferrule 324 while leaving ferrule 324 within ferrule receptacle 334, as shown in FIG. 34E, and such as described hereinabove with reference to FIG. 5G. During this first portion of the proximal withdrawal, proximal spring 1668 applies a portion of the elastic potential energy stored therein to shaft engagement surface 1656 of needle-control shaft 1660, thereby causing a first portion of a proximal stroke of needle-control shaft 1660. At a point during this first portion of the proximal stroke, shown in FIG. 34D, a proximally-facing needle-pushing surface 1661 of needle-control shaft 1660 comes in contact with and proximally pushes distally-facing surface 463 of first ferrule-advancing suturing needle 336, 336A, thereby proximally withdrawing first ferrule-advancing suturing needle 336, 336A. During this first portion of the proximal stroke, distal spring 1670 remains axially compressed and distal slider 1666 remains maximally distally advanced, such that suture-positioning support 1630 remains laterally extended in deployed position 358.

As shown in the transition between FIGS. 34E and 34F, during a second portion of the proximal withdrawal, proximal spring 1668 continues to apply the elastic potential energy stored therein to shaft engagement surface 1656 of needle-control shaft 1660, thereby causing a second portion of a proximal stroke of needle-control shaft 1660. As proximal spring 1668 applies less force to distal slider 1666, distal spring 1670 applies the elastic potential energy stored therein to distal slider 1666, causing the distal slider to return proximally to its initial position. This distal motion of distal slider 1666 proximally withdraws extension-control-shaft 1642, thereby transitioning suture-positioning support 1630 from deployed position 358 back to delivery position 356, as shown in FIG. 34F, and such as described hereinabove with reference to FIG. 5G.

Typically, support-and-needle user control 434 is partially proximally released to cause the transition between FIG. 34C and FIG. 34D, further proximally released to cause the subsequent transition between FIG. 34D and FIG. 34E, and still further proximally released to cause the subsequent transition between FIG. 34E and FIG. 34F.

The remaining steps of the procedure using control handle 1640 are performed as described hereinabove with reference to FIGS. 7F-H, mutatis mutandis.

In some configurations of the closure devices described hereinabove, each of the ferrules is described as being coupled to a distal end portion of a suture that is distally advanced to a ferrule receptacle of a suture-positioning support using a ferrule-advancing suturing needle. By contrast, in other applications of the present invention, the ferrule is not initially coupled to a distal end portion of a suture that is distally advanced to the ferrule receptacle. Instead, for example, the ferrule may be coupled to an end portion of a suture segment that is prepositioned within the suture-positioning support. The ferrule receptacle of the suture-positioning support is configured to removably receive the ferrule before the suture-positioning support is inserted into the hollow anatomical structure, such as during assembly of the closure device. The ferrule-withdrawing suturing needle is configured to be coupled to the ferrule that was pre-positioned in the ferrule receptacle, and, upon proximal withdrawal of the ferrule-withdrawing suturing needle coupled to the ferrule, to remove the ferrule from the ferrule receptacle, thereby proximally pulling the suture segment.

For some applications, the above-mentioned ferrule that is coupled to the end portion of the suture segment is a first ferrule, and a second ferrule is coupled to the other end portion of the suture segment that is prepositioned within the suture-positioning support. The closure device comprises, in addition to the above-mentioned ferrule-withdrawing suturing needle, a suture-advancing suturing needle. The closure device is configured to distally advance the suture-advancing suturing needle while coupled to a suture that is initially separate from the above-mentioned suture segment, such that the suture-advancing suturing needle couples the suture to the second ferrule, thereby coupling the suture to the suture segment. Optionally, the suture comprises a barbed tip that is configured to engage the second ferrule, rather than the suture-advancing suturing needle engaging the ferrule. Optionally, some or all of the needles are hollow along at least a portion of the needles, and some or all of the sutures pass through the hollow portions of the needles.

Although the closure devices have been generally described hereinabove as being used for suturing a puncture through a wall of a blood vessel, the closure devices may alternatively be used to suture internal punctures or wounds or external punctures or wounds, such as an endoscopic puncture through a wall of a body cavity, or for skin closure for orthopedic procedures, such as arthroplasty. The closure devices may also be used for intracorporeal suturing, such as during minimally invasive surgery, such as laparoscopic procedures; for these applications, the closure devices may be implemented as a laparoscopic suturing tool. The closure devices may also be used to secure a lead or catheter to tissue for lead or catheter anchoring.

In an embodiment, the techniques and apparatus described herein are combined with techniques and apparatus described in one or more of the following patent applications, which are assigned to the assignee of the present application and are incorporated herein by reference:
U.S. Provisional Application 63/404,630, filed Sep. 8, 2022
U.S. Provisional Application 63/526,765, filed Jul. 14, 2023
International Application PCT/IL 2023/050966, filed Sep. 7, 2023
U.S. application Ser. No. 18/517,764, filed Nov. 22, 2023, which published as US Patent Application Publication 2024/0081800 to Iamberger et al.

U.S. Provisional Application 63/564,785, filed Mar. 13, 2024

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A closure device for deploying sutures, the closure device comprising:
a suture;
an elongate support; and
a suture-positioning support, which is coupled to a distal end portion of the elongate support, and which is configured to assume:
(i) a delivery position, in which a suture-positioning support axis of the suture-positioning support (a) forms a first angle of less than 45 degrees with a distal-support central longitudinal axis of the distal end portion of the elongate support, or (b) is parallel with the distal-support central longitudinal axis, and
(ii) a plurality of deployed positions, in which the suture-positioning support is laterally extended with respect to the distal end portion of the elongate support such that the suture-positioning support axis forms a second angle of at least 60 degrees with the distal-support central longitudinal axis,
wherein the closure device is configured to:
transition the suture-positioning support from the delivery position to a first one of the deployed positions, in which the suture-positioning support laterally extends in a first direction from the distal end portion of the elongate support,
couple the suture to the suture-positioning support while the suture-positioning support is in the first deployed position,
thereafter, transition the suture-positioning support from the first deployed position back to the delivery position,
thereafter, rotate the suture-positioning support, while in the delivery position, about a distal-support central longitudinal axis of the distal portion of the elongate support,
thereafter, transition the suture-positioning support from the delivery position to a second one of the deployed positions, in which the suture-positioning support laterally extends in a second direction from the distal end portion of the elongate support, the second direction different from the first direction, and
proximally withdraw the suture from the suture-positioning support while the suture-positioning support is in the second deployed position.

2. The closure device according to claim 1, wherein the first angle is less than 30 degrees, and an external portion of the suture-positioning support that interfaces with the distal end portion of the elongate support is shaped so as to define two partial spherical surfaces.

3. The closure device according to claim 1, wherein the closure device is configured to close a puncture, which is a puncture through a wall of a blood vessel or an endoscopic puncture through a wall of a body cavity.

4. The closure device according to claim 1, wherein the elongate support comprises one or more shafts, and wherein the elongate support is configured to rotate the suture-positioning support about the distal-support central longitudinal axis by rotating at least one of the one or more of the shafts of the elongate support.

5. The closure device according to claim 1,
wherein the closure device further comprises:
a ferrule, which is coupled to a distal end portion of the suture;
a ferrule-advancing suturing needle, which is removably couplable to the ferrule; and
a ferrule-withdrawing suturing needle,
wherein the suture-positioning support is shaped so as to define a ferrule receptacle, which is configured to removably receive the ferrule,
wherein the elongate support defines, through a longitudinal portion of the elongate support, first and second needle lumens having first and second distal lumen openings, respectively, and
wherein the closure device is configured to:
couple the suture to the suture-positioning support by distally advancing, within the first needle lumen and out of the first distal lumen opening, the ferrule-advancing suturing needle removably coupled to the ferrule, and inserting the ferrule into the ferrule receptacle, and
proximally withdraw the suture from the suture-positioning support by distally advancing, within the second needle lumen and out of the second distal lumen opening, the ferrule-withdrawing suturing needle to the ferrule, coupling the ferrule-withdrawing suturing needle to the ferrule, and proximally withdrawing the ferrule-withdrawing suturing needle while coupled to the ferrule, so as to remove the ferrule from the ferrule receptacle.

6. The closure device according to claim 5,
wherein a free end of the suture-positioning support near which the ferrule receptacle is disposed is shaped so as to define a wall, and
wherein the ferrule receptacle is shaped so as to define a slit through the wall of the suture-positioning support.

7. The closure device according to claim 6, wherein the wall is shaped so as to define a recessed portion along one side of the slit, thereby enlarging a longitudinal portion of the slit, which allows the suture to exit the ferrule receptacle during rotation of the suture-positioning support about the distal-support central longitudinal axis.

8. The closure device according to claim 7, wherein the closure device is configured to comprise at least one of the following:
(a) the recessed portion extends along ⅓-⅔ of a length of the slit;
(b) the recessed portion defines a flat surface that is angled with respect to a remainder of the wall or defines a curved surface;
(c) the wall is shaped so as to define exactly one recessed portion along the one side of the slit, and the one side is a trailing side of the slit during rotation of the suture-positioning support about the distal-support central longitudinal axis by the closure device.

9. The closure device according to claim 7, wherein the wall is shaped so as to define two recessed portions respectively along both sides of the slit.

10. The closure device according to claim 1,
wherein the elongate support comprises an outer tubular shaft having a distal end,
wherein the suture-positioning support has proximal and distal end portions at opposite ends of the suture-positioning support,
wherein the proximal end portion of the suture-positioning support is coupled to the elongate support, and
wherein the distal end portion of the suture-positioning support is disposed distally to the distal end of the outer tubular shaft.

11. The closure device according to claim 10, wherein the elongate support further comprises an inner shaft nested within the outer tubular shaft, and wherein the suture-positioning support is coupled to the inner shaft of the elongate support.

12. The closure device according to claim 1,
wherein the suture-positioning support is pivotably coupled to the distal end portion of the elongate support such that the suture-positioning support is rotatable about a pivot axis that is not coaxial with the distal-support central longitudinal axis,
wherein the closure device is configured to transition the suture-positioning support from the delivery position to the first deployed position by rotating the suture-positioning support about the pivot axis; and
wherein the closure device is configured to transition the suture-positioning support from the first deployed position back to the delivery position by rotating the suture-positioning support about the pivot axis.

13. A closure device for deploying sutures, the closure device comprising:
a suture;
a ferrule, coupled to an end portion of the suture;
an elongate support, which comprises one or more shafts;
a suture-positioning support, which is (a) coupled to a distal end portion of the elongate support, (b) laterally extendable with respect to the elongate support, and (c) shaped so as to define a ferrule receptacle, which is configured to removably receive the ferrule;
a control handle, which (a) is coupled to a proximal end portion of the elongate support, and (b) comprises a wall shaped so as to define a lateral opening through the wall; and
a ferrule-withdrawing suturing needle, which (a) includes a distal end portion that is removably couplable to the ferrule, and (b) is removably disposed entirely within the control handle,
wherein the closure device is configured such that:
distal advancement of the ferrule-withdrawing suturing needle to the ferrule in the ferrule receptacle couples the ferrule-withdrawing suturing needle to the ferrule,
proximal withdrawal of the ferrule-withdrawing suturing needle coupled to the ferrule removes the ferrule from the ferrule receptacle, and
the ferrule-withdrawing suturing needle is removable from the control handle via the lateral opening.

14. The closure device according to claim 13, wherein the ferrule is coupled to a distal end portion of the suture, and the lateral opening is disposed distal to a proximal end portion of the control handle.

15. The closure device according to claim 13, further comprising a ferrule-advancing suturing needle, which (a) includes a distal portion that is removably couplable to the ferrule, and (b) is removably disposed entirely within the control handle,
wherein the closure device is configured to direct the ferrule into the ferrule receptacle during distal advancement of the suturing ferrule-advancing needle removably coupled to the ferrule.

16. The closure device according to claim 13, wherein the closure device is configured such that the control handle prevents removal of the ferrule-withdrawing suturing needle from the control handle until after the proximal withdrawal of the ferrule-withdrawing suturing needle removes the ferrule from the ferrule receptacle.

17. The closure device according to claim 13, wherein the ferrule-withdrawing suturing needle comprises a needle handle, which is moveable separately from the needle user control, and which coupled to a proximal end of the ferrule-withdrawing suturing needle, wherein the ferrule-withdrawing suturing needle, including the needle handle and the distal end portion of the ferrule-withdrawing suturing needle, is removably disposed entirely within the control handle, and
wherein the control handle is configured to at least partially eject the needle handle from the lateral opening after the proximal withdrawal of the ferrule-withdrawing suturing needle removes the ferrule from the ferrule receptacle.

18. The closure device according to claim 13, wherein the control handle comprises a needle user control that is permanently coupled to the control handle, and wherein the control handle is configured such that:
actuation of the needle user control causes the distal advancement of the ferrule-withdrawing suturing needle to the ferrule in the ferrule receptacle, and
subsequent actuation of the needle user control causes the proximal withdrawal of the ferrule-withdrawing suturing needle coupled to the ferrule.

19. The closure device according to claim 18, wherein the ferrule-withdrawing suturing needle comprises a needle handle coupled to a proximal end of the ferrule-withdrawing suturing needle, wherein the ferrule-withdrawing suturing needle, including the needle handle and the distal end portion of the ferrule-withdrawing suturing needle, is removably disposed entirely within the control handle;
wherein the subsequent actuation of the needle user control is caused by releasing the user control; and
wherein the needle user control comprises a support-and-needle user control that is permanently coupled to the control handle, and wherein the control handle is configured such that:
the actuation of the support-and-needle user control causes (a) the distal advancement of the ferrule-withdrawing suturing needle to the ferrule in the ferrule receptacle, and (b) lateral extension of the suture-positioning support, with respect to the elongate support, from a delivery position to a deployed position, and
the subsequent actuation of the support-and-needle user control causes (a) the proximal withdrawal of the ferrule-withdrawing suturing needle coupled to the ferrule, and (b) transitioning of the suture-positioning support from the deployed position back to the delivery position.

20. Apparatus comprising:
a suture;
a ferrule-withdrawing suturing needle;
a ferrule, which (a) is coupled to a distal end portion of the suture, and (b) is shaped so as to define (i) a ferrule lumen and (ii) one or more tabs, which are biased to protrude radially inward within the ferrule lumen and to engage the ferrule-withdrawing suturing needle upon insertion of the ferrule-withdrawing suturing needle into an end opening of the ferrule lumen, thereby inhibiting withdrawal of the ferrule-withdrawing suturing needle from the ferrule lumen; and
a ferrule-advancing suturing needle, which includes a distal end portion that is shaped and sized so as, upon insertion of the distal end portion into the ferrule lumen, to provide space within the ferrule lumen for the one or more tabs, and to contact an internal surface of the ferrule lumen so as to grasp the ferrule.
